SEQUENCE LISTING (248 sequences)

SEQ ID NO: 1 - DNA, 2665 bp, Homo sapiens
  CDS (236)..(1849)
gtcgactggaggcccagctgcccgtcatcagagtgacaggtcttatgacagcctgattggtgactcgggctgggtgtggattctcaccccaggcctctgcctgctttctcagaccctcatctgtcacccccacgctgaacccagctgccacccccagaagcccatcagactgcccccagcacacggaatggatttctgagaaagaagccgaaacagaaggcccgtgggagtcagcatgccgcgtggctgggccgcccccttgctcctgctgctgctccagggaggctggggctgccccgacctcgtctgctacaccgattacctccagacggtcatctgcatcctggaaatgtggaacctccaccccagcacgctcacccttacctggcaagaccagtatgaagagctgaaggacgaggccacctcctgcagcctccacaggtcggcccacaatgccacgcatgccacctacacctgccacatggatgtattccacttcatggccgacgacattttcagtgtcaacatcacagaccagtctggcaactactcccaggagtgtggcagctttctcctggctgagagcatcaagccggctccccctttcaacgtgactgtgaccttctcaggacagtataatatctcctggcgctcagattacgaagaccctgccttctacatgctgaagggcaagcttcagtatgagctgcagtacaggaaccggggagacccctgggctgtgagtccgaggagaaagctgatctcagtggactcaagaagtgtctccctcctccccctggagttccgcaaagactcgagctatgagctgcaggtgcgggcagggcccatgcctggctcctcctaccaggggacctggagtgaatggagtgacccggtcatctttcagacccagtcagaggagttaaaggaaggctggaaccctcacctgctgcttctcctcctgcttgtcatagtcttcattcctgccttctggagcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagctaatgaggctgactggatgtccagagctggccaggccactgggccctgagccagagacaaggtcacctgggctgtgatgtgaagacacctgcagcctttggtctcctggatgggcctttgagcctgatgtttacagtgtctgtgtgtgtgtgtgcatatgtgtgtgtgtgcatatgcatgtgtgtgtgtgtgtgtgtcttaggtgcgcagtggcatgtccacgtgtgtgtgtgattgcacgtgcctgtgggcctgggataatgcccatggtactccatgcattcacctgccctgtgcatgtctggactcacggagctcacccatgtgcacaagtgtgcacagtaaacgtgtttgtggtcaacagatgacaacagccgtcctccctcctagggtcttgtgttgcaagttggtccacagcatctccggggctttgtgggatcagggcattgcctgtgactgaggcggagcccagccctccagcgtctgcctccaggagctgcaagaagtccatattgttccttatcacctgccaacaggaagcgaaaggggatggagtgagcccatggtgacctcgggaatggcaattttttgggcggcccctggacgaaggtctgaatcccgactctgataccttctggctgtgctacctgagccaagtcgcctcccctctctgggctagagtttccttatccagacagtggggaaggcatgacacacctgggggaaattggcgatgtcacccgtgtacggtacgcagcccagagcagaccctcaataaacgtcagcttccttcaaaaaaaaaaaaaaaaatctaga

SEQ ID NO: 2 - protein, 538 aa, Homo sapiens
MPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 3 - DNA, 2628 bp, Mus musculus
  CDS (407)..(1993)
gtcgacgcggcggtaccagctgtctgcccacttctcctgtggtgtgcctcacggtcacttgcttgtctgaccgcaagtctgcccatccctggggcagccaactggcctcagcccgtgccccaggcgtgccctgtctctgtctggctgccccagccctactgtcttcctctgtgtaggctctgcccagatgcccggctggtcctcagcctcaggactatctcagcagtgactcccctgattctggacttgcacctgactgaactcctgcccacctcaaaccttcacctcccaccaccaccactccgagtcccgctgtgactcccacgcccaggagaccacccaagtgccccagcctaaagaatggctttctgagaaagaccctgaaggagtaggtctgggacacagcatgccccggggcccagtggctgccttactcctgctgattctccatggagcttggagctgcctggacctcacttgctacactgactacctctggaccatcacctgtgtcctggagacacggagccccaaccccagcatactcagtctcacctggcaagatgaatatgaggaacttcaggaccaagagaccttctgcagcctacacaggtctggccacaacaccacacatatatggtacacgtgccatatgcgcttgtctcaattcctgtccgatgaagttttcattgtcaatgtgacggaccagtctggcaacaactcccaagagtgtggcagctttgtcctggctgagagcatcaaaccagctccccccttgaacgtgactgtggccttctcaggacgctatgatatctcctgggactcagcttatgacgaaccctccaactacgtgctgaggggcaagctacaatatgagctgcagtatcggaacctcagagacccctatgctgtgaggccggtgaccaagctgatctcagtggactcaagaaacgtctctcttctccctgaagagttccacaaagattctagctaccagctgcaggtgcgggcagcgcctcagccaggcacttcattcagggggacctggagtgagtggagtgaccccgtcatctttcagacccaggctggggagcccgaggcaggctgggaccctcacatgctgctgctcctggctgtcttgatcattgtcctggttttcatgggtctgaagatccacctgccttggaggctatggaaaaagatatgggcaccagtgcccacccctgagagtttcttccagcccctgtacagggagcacagcgggaacttcaagaaatgggttaatacccctttcacggcctccagcatagagttggtgccacagagttccacaacaacatcagccttacatctgtcattgtatccagccaaggagaagaagttcccggggctgccgggtctggaagagcaactggagtgtgatggaatgtctgagcctggtcactggtgcataatccccttggcagctggccaagcggtctcagcctacagtgaggagagagaccggccatatggtctggtgtccattgacacagtgactgtgggagatgcagagggcctgtgtgtctggccctgtagctgtgaggatgatggctatccagccatgaacctggatgctggccgagagtctggccctaattcagaggatctgctcttggtcacagaccctgcttttctgtcttgcggctgtgtctcaggtagtggtctcaggcttggaggctccccaggcagcctactggacaggttgaggctgtcatttgcaaaggaaggggactggacagcagacccaacctggagaactgggtccccaggagggggctctgagagtgaagcaggttccccccctggtctggacatggacacatttgacagtggctttgcaggttcagactgtggcagccccgtggagactgatgaaggaccccctcgaagctatctccgccagtgggtggtcaggacccctccacctgtggacagtggagcccagagcagctagcatataataaccagctatagtgagaagaggcctctgagcctggcatttacagtgtgaacatgtaggggtgtgtgtgtgtgtgtgtgtgtgtgtgtgtgtgtgtgtgtgtgtgtgtgtgtgtgtgtgtcttgggttgtgtgttagcacatccatgttgggatttggtctgttgctatgtattgtaatgctaaattctctacccaaagttctaggcctacgagtgaattctcatgtttacaaacttgctgtgtaaaccttgttccttaatttaataccattggttaaataaaattggctgcaaccaattactggagggattagaggtagggggcttttgagttacctgtttggagatggagaaggagagaggagagaccaagaggagaaggaggaaggagaggagaggagaggagaggagaggagaggagaggagaggagaggagaggagaggagaggctgccgtgaggggagagggaccatgagcctgtggccaggagaaacagcaagtatctggggtacactggtgaggaggtggccaggccagcagttagaagagtagattaggggtgacctccagtatttgtcaaagccaattaaaataacaaaaaaaaaaaaaaagcggccgctctaga

SEQ ID NO: 4 - protein, 529 aa, Mus musculus
MPRGPVAALLLLILHGAWSCLDLTCYTDYLWTITCVLETRSPNPSILSLTWQDEYEELQDQETFCSLHRSGHNTTHIWYTCHMRLSQFLSDEVFIVNVTDQSGNNSQECGSFVLAESIKPAPPLNVTVAFSGRYDISWDSAYDEPSNYVLRGKLQYELQYRNLRDPYAVRPVTKLISVDSRNVSLLPEEFHKDSSYQLQVRAAPQPGTSFRGTWSEWSDPVIFQTQAGEPEAGWDPHMLLLLAVLIIVLVFMGLKIHLPWRLWKKIWAPVPTPESFFQPLYREHSGNFKKWVNTPFTASSIELVPQSSTTTSALHLSLYPAKEKKFPGLPGLEEQLECDGMSEPGHWCIIPLAAGQAVSAYSEERDRPYGLVSIDTVTVGDAEGLCVWPCSCEDDGYPAMNLDAGRESGPNSEDLLLVTDPAFLSCGCVSGSGLRLGGSPGSLLDRLRLSFAKEGDWTADPTWRTGSPGGGSESEAGSPPGLDMDTFDSGFAGSDCGSPVETDEGPPRSYLRQWVVRTPPPVDSGAQSS

SEQ ID NO: 5 - DNA, 354 bp, Homo sapiens
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagt

SEQ ID NO: 6 - protein, 118 aa, Homo sapiens
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSS

SEQ ID NO: 7 - DNA, 354 bp, Homo sapiens
caggtgcagctgcaggagtctggccctggcctggtgaagccttccgagaccctgtctctgacctgtgccgtgtccggctactccatctcctccggctactactggggctggatcagacagcctcctggcaagggcctggagtggatcggctccatctctcacaccggcaacacctactacaacccccctctgaagtccagagtgaccatctccgtggacacctccaagaaccagttctccctgaagctgtcctctgtgaccgctgccgataccgccgtgtactactgtgccagaggcggcggaatctccagacctgagtactggggccagggcaccctggtgaccgtgtcctct

SEQ ID NO: 8 - protein, 118 aa, Homo sapiens
QVQLQESGPGLVKPSETLSLTCAVSGYSISSGYYWGWIRQPPGKGLEWIGSISHTGNTYYNPPLKSRVTISVDTSKNQFSLKLSSVTAADTAVYYCARGGGISRPEYWGQGTLVTVSS

SEQ ID NO: 9 - DNA, 327 bp, Homo sapiens
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtaactcccgggactccagtggcaacccccatgttctgttcggcggagggacccagctcaccgtttta

SEQ ID NO: 10 - protein, 109 aa, Homo sapiens
SSELTQDPPVSVALGQTVTLTCQGDSLRTYYASWYQQKSGQAPILLLYGKHKRPSGIPDRFSGSTSGDTASLTITGAQAEDEADYYCNSRDSSGNPHVLFGGGTQLTVL

SEQ ID NO: 11 - DNA, 327 bp, Homo sapiens
tcctctgagctgacccaggatcctgctgtgtctgtggccctgggccagaccgtcaggatcacctgccagggcgatagcctgagaacctactacgcctcctggtatcagcagaagcctggacaggcccctgtgctggtgatctacggcaagcacaagaggccatccggcatccctgacagattctccggctcctcctctggcaataccgcctccctgaccatcaccggcgctcaggccgaggacgaggccgactactactgtaactcccgggactcttccggcaaccctcacgtgctgtttggcggcggaacccagctgaccgtgcta

SEQ ID NO: 12 - protein, 109 aa, Homo sapiens
SSELTQDPAVSVALGQTVRITCQGDSLRTYYASWYQQKPGQAPVLVIYGKHKRPSGIPDRFSGSSSGNTASLTITGAQAEDEADYYCNSRDSSGNPHVLFGGGTQLTVL

SEQ ID NO: 13 - DNA, 354 bp, artificial
  H3 VH
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgattcatggggttcggccgcccggagtactggggcaaaggcaccctggtcaccgtctcgagt

SEQ ID NO: 14 - protein, 118 aa, artificial
  H3 VH
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARFMGFGRPEYWGKGTLVTVSS

SEQ ID NO: 15 - DNA, 354 bp, artificial
  H4 VH
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgatggctcgggttcggccgcccggagtactggggcaaaggcaccctggtcaccgtctcgagt

SEQ ID NO: 16 - protein, 118 aa, artificial
  H4 VH
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARWLGFGRPEYWGKGTLVTVSS

SEQ ID NO: 17 - DNA, 354 bp, artificial
  H5 VH
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgattcttgggcttcggccggccggagtactggggcaaaggcaccctggtcaccgtctcgagt

SEQ ID NO: 18 - protein, 118 aa, artificial
  H5 VH
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARFLGFGRPEYWGKGTLVTVSS

SEQ ID NO: 19 - DNA, 354 bp, artificial
  H6 VH
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgattcttcggcttcggccgcccggagtactggggcaaaggcaccctggtcaccgtctcgagt

SEQ ID NO: 20 - protein, 118 aa, artificial
  H6 VH
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARFFGFGRPEYWGKGTLVTVSS

SEQ ID NO: 21 - DNA, 327 bp, artificial
  L1 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgcgtcccggtcggtgagcggcaacccccatgttctgttcggcggagggacccagctcaccgtttta

SEQ ID NO: 22 - protein, 109 aa, artificial
  L1 VL
SSELTQDPPVSVALGQTVTLTCQGDSLRTYYASWYQQKSGQAPILLLYGKHKRPSGIPDRFSGSTSGDTASLTITGAQAEDEADYYCASRSVSGNPHVLFGGGTQLTVL

SEQ ID NO: 23 - DNA, 327 bp, artificial
  L2 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgtcgcccggtcggtggtgggcaacccccatgttctgttcggcggagggacccagctcaccgtttta

SEQ ID NO: 24 - protein, 109 aa, artificial
  L2 VL
SSELTQDPPVSVALGQTVTLTCQGDSLRTYYASWYQQKSGQAPILLLYGKHKRPSGIPDRFSGSTSGDTASLTITGAQAEDEADYYCVARSVVGNPHVLFGGGTQLTVL

SEQ ID NO: 25 - DNA, 327 bp, artificial
  L3 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgtcagcagggcggtggtgggcaacccccatgttctgttcggcggagggacccagctcaccgtttta

SEQ ID NO: 26 - protein, 109 aa, artificial
  L3 VL
SSELTQDPPVSVALGQTVTLTCQGDSLRTYYASWYQQKSGQAPILLLYGKHKRPSGIPDRFSGSTSGDTASLTITGAQAEDEADYYCVSRAVVGNPHVLFGGGTQLTVL

SEQ ID NO: 27 - DNA, 327 bp, Artificial
  L4 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtagcacccgcagcagcaagggcaacccccatgttctgttcggcggagggacccagctcaccgtttta

SEQ ID NO: 28 - protein, 109 aa, artificial
  L4 VL
SSELTQDPPVSVALGQTVTLTCQGDSLRTYYASWYQQKSGQAPILLLYGKHKRPSGIPDRFSGSTSGDTASLTITGAQAEDEADYYCSTRSSKGNPHVLFGGGTQLTVL

SEQ ID NO: 29 - DNA, 327 bp, artificial
  L5 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgccagcaggtcctccaagggcaacccccatgttctgttcggcggagggacccagctcaccgtttta

SEQ ID NO: 30 - protein, 109 aa, artificial
  L5 VL
SSELTQDPPVSVALGQTVTLTCQGDSLRTYYASWYQQKSGQAPILLLYGKHKRPSGIPDRFSGSTSGDTASLTITGAQAEDEADYYCASRSSKGNPHVLFGGGTQLTVL

SEQ ID NO: 31 - DNA, 327 bp, artificial
  L6 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtatgagcaggagcatctggggcaacccccatgttctgttcggcggagggacccagctcaccgtttta

SEQ ID NO: 32 - protein, 109 aa, artificial
  L6 VL
SSELTQDPPVSVALGQTVTLTCQGDSLRTYYASWYQQKSGQAPILLLYGKHKRPSGIPDRFSGSTSGDTASLTITGAQAEDEADYYCMSRSIWGNPHVLFGGGTQLTVL

SEQ ID NO: 33 - DNA, 327 bp, artificial
  L8 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtaccacgcgctccacccagggcaacccccatgttctgttcggcggagggacccagctcaccgtttta

SEQ ID NO: 34 - protein, 109 aa, artificial
  L8 VL
SSELTQDPPVSVALGQTVTLTCQGDSLRTYYASWYQQKSGQAPILLLYGKHKRPSGIPDRFSGSTSGDTASLTITGAQAEDEADYYCTTRSTQGNPHVLFGGGTQLTVL

SEQ ID NO: 35 - DNA, 327 bp, artificial
  L9 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgtcgccaggtccaacaagggcaacccccatgttctgttcggcggagggacccagctcaccgtttta

SEQ ID NO: 36 - protein, 109 aa, artificial
  L9 VL
SSELTQDPPVSVALGQTVTLTCQGDSLRTYYASWYQQKSGQAPILLLYGKHKRPSGIPDRFSGSTSGDTASLTITGAQAEDEADYYCVARSNKGNPHVLFGGGTQLTVL

SEQ ID NO: 37 - DNA, 327 bp, artificial
  L10 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtatcagccggtcgatctacggcaacccccatgttctgttcggcggagggacccagctcaccgtttta

SEQ ID NO: 38 - protein, 109 aa, artificial
  L10 VL
SSELTQDPPVSVALGQTVTLTCQGDSLRTYYASWYQQKSGQAPILLLYGKHKRPSGIPDRFSGSTSGDTASLTITGAQAEDEADYYCISRSIYGNPHVLFGGGTQLTVL

SEQ ID NO: 39 - DNA, 327 bp, artificial
  L11 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgttcctcccgctcccgccacggcaacccccatgttctgttcggcggagggacccagctcaccgtttta

SEQ ID NO: 40 - protein, 109 aa, artificial
  L11 VL
SSELTQDPPVSVALGQTVTLTCQGDSLRTYYASWYQQKSGQAPILLLYGKHKRPSGIPDRFSGSTSGDTASLTITGAQAEDEADYYCSSRSRHGNPHVLFGGGTQLTVL

SEQ ID NO: 41 - DNA, 327 bp, artificial
  L12 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgtcgcgagggggacgaggggcaacccccatgttctgttcggcggagggacccagctcaccgtttta

SEQ ID NO: 42 - protein, 109 aa, artificial
  L12 VL
SSELTQDPPVSVALGQTVTLTCQGDSLRTYYASWYQQKSGQAPILLLYGKHKRPSGIPDRFSGSTSGDTASLTITGAQAEDEADYYCVARGTRGNPHVLFGGGTQLTVL

SEQ ID NO: 43 - DNA, 327 bp, artificial
  L13 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgtcacccgcaaccgctacggcaacccccatgttctgttcggcggagggacccagctcaccgtttta

SEQ ID NO: 44 - protein, 109 aa, artificial
  L13 VL
SSELTQDPPVSVALGQTVTLTCQGDSLRTYYASWYQQKSGQAPILLLYGKHKRPSGIPDRFSGSTSGDTASLTITGAQAEDEADYYCVTRNRYGNPHVLFGGGTQLTVL

SEQ ID NO: 45 - DNA, 327 bp, artificial
  L14 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtatggcgaggtcgaggaagggcaacccccatgttctgttcggcggagggacccagctcaccgtttta

SEQ ID NO: 46 - protein, 109 aa, artificial
  L14 VL
SSELTQDPPVSVALGQTVTLTCQGDSLRTYYASWYQQKSGQAPILLLYGKHKRPSGIPDRFSGSTSGDTASLTITGAQAEDEADYYCMARSRKGNPHVLFGGGTQLTVL

SEQ ID NO: 47 - DNA, 327 bp, artificial
  L15 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgttccacccgcgccatccacggcaacccccatgttctgttcggcggagggacccagctcaccgtttta

SEQ ID NO: 48 - protein, 109 aa, artificial
  L15 VL
SSELTQDPPVSVALGQTVTLTCQGDSLRTYYASWYQQKSGQAPILLLYGKHKRPSGIPDRFSGSTSGDTASLTITGAQAEDEADYYCSTRAIHGNPHVLFGGGTQLTVL

SEQ ID NO: 49 - DNA, 327 bp, artificial
  L16 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgtgacgaggagcgcgaagggcaacccccatgttctgttcggcggagggacccagctcaccgtttta

SEQ ID NO: 50 - protein, 109 aa, artificial
  L16 VL
SSELTQDPPVSVALGQTVTLTCQGDSLRTYYASWYQQKSGQAPILLLYGKHKRPSGIPDRFSGSTSGDTASLTITGAQAEDEADYYCVTRSAKGNPHVLFGGGTQLTVL

SEQ ID NO: 51 - DNA, 327 bp, artificial
  L17 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtagcacgaggtcgaggaagggcaacccccatgttctgttcggcggagggacccagctcaccgtttta

SEQ ID NO: 52 - protein, 109 aa, artificial
  L17 VL
SSELTQDPPVSVALGQTVTLTCQGDSLRTYYASWYQQKSGQAPILLLYGKHKRPSGIPDRFSGSTSGDTASLTITGAQAEDEADYYCSTRSRKGNPHVLFGGGTQLTVL

SEQ ID NO: 53 - DNA, 327 bp, artificial
  L18 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgtcacgaggagcgtgaagggcaacccccatgttctgttcggcggagggacccagctcaccgtttta

SEQ ID NO: 54 - protein, 109 aa, artificial
  L18 VL
SSELTQDPPVSVALGQTVTLTCQGDSLRTYYASWYQQKSGQAPILLLYGKHKRPSGIPDRFSGSTSGDTASLTITGAQAEDEADYYCVTRSVKGNPHVLFGGGTQLTVL

SEQ ID NO: 55 - DNA, 327 bp, artificial
  L19 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgtcgcgcgggcggtgaggggcaacccccatgttctgttcggcggagggacccagctcaccgtttta

SEQ ID NO: 56 - protein, 109 aa, artificial
  L19 VL
SSELTQDPPVSVALGQTVTLTCQGDSLRTYYASWYQQKSGQAPILLLYGKHKRPSGIPDRFSGSTSGDTASLTITGAQAEDEADYYCVARAVRGNPHVLFGGGTQLTVL

SEQ ID NO: 57 - DNA, 327 bp, artificial
  L20 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgtctcccgcagcgcgaagggcaacccccatgttctgttcggcggagggacccagctcaccgtttta

SEQ ID NO: 58 - protein, 109 aa, artificial
  L20 VL
SSELTQDPPVSVALGQTVTLTCQGDSLRTYYASWYQQKSGQAPILLLYGKHKRPSGIPDRFSGSTSGDTASLTITGAQAEDEADYYCVSRSAKGNPHVLFGGGTQLTVL

SEQ ID NO: 59 - DNA, 327 bp, artificial
  L21 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgccacccgggcggtccggggcaacccccatgttctgttcggcggagggacccagctcaccgtttta

SEQ ID NO: 60 - protein, 109 aa, artificial
  L21 VL
SSELTQDPPVSVALGQTVTLTCQGDSLRTYYASWYQQKSGQAPILLLYGKHKRPSGIPDRFSGSTSGDTASLTITGAQAEDEADYYCATRAVRGNPHVLFGGGTQLTVL

SEQ ID NO: 61 - DNA, 327 bp, artificial
  L23 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgttcggcgcggtcggtgaggggcaacccccatgttctgttcggcggagggacccagctcaccgtttta

SEQ ID NO: 62 - protein, 109 aa, artificial
  L23 VL
SSELTQDPPVSVALGQTVTLTCQGDSLRTYYASWYQQKSGQAPILLLYGKHKRPSGIPDRFSGSTSGDTASLTITGAQAEDEADYYCSARSVRGNPHVLFGGGTQLTVL

SEQ ID NO: 63 - DNA, 327 bp, artificial
  L24 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtatcgccaggagcaacaagggcaacccccatgttctgttcggcggagggacccagctcaccgtttta

SEQ ID NO: 64 - protein, 109 aa, artificial
  L24 VL
SSELTQDPPVSVALGQTVTLTCQGDSLRTYYASWYQQKSGQAPILLLYGKHKRPSGIPDRFSGSTSGDTASLTITGAQAEDEADYYCIARSNKGNPHVLFGGGTQLTVL

SEQ ID NO: 65 - DNA, 327 bp, artificial
  L25 VL
tcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtacgacgcggagcaacaagggcaacccccatgttctgttcggcggagggacccagctcaccgtttta

SEQ ID NO: 66 - protein, 109 aa, artificial
  L25 VL
SSELTQDPPVSVALGQTVTLTCQGDSLRTYYASWYQQKSGQAPILLLYGKHKRPSGIPDRFSGSTSGDTASLTITGAQAEDEADYYCTTRSNKGNPHVLFGGGTQLTVL

SEQ ID NO: 67 - DNA, 1401 bp, artificial
  VH4 heavy chain
atgggatggagctgtatcatcctcttcttggtagcaacagctacaggcgcgcactctcaggtgcagctgcaggagtctggccctggcctggtgaagccttccgagaccctgtctctgacctgtgccgtgtccggctactccatctcctccggctactactggggctggatcagacagcctcctggcaagggcctggagtggatcggctccatctctcacaccggcaacacctactacaacccccctctgaagtccagagtgaccatctccgtggacacctccaagaaccagttctccctgaagctgtcctctgtgaccgctgccgataccgccgtgtactactgtgccagatggctcgggttcggccgcccggagtactggggcaaaggcaccctggtgaccgtgtcctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 68 - protein, 467 aa, artificial
  VH4 heavy chain
MGWSCIILFLVATATGAHSQVQLQESGPGLVKPSETLSLTCAVSGYSISSGYYWGWIRQPPGKGLEWIGSISHTGNTYYNPPLKSRVTISVDTSKNQFSLKLSSVTAADTAVYYCARWLGFGRPEYWGKGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - DNA, 1401 bp, artificial
  VH5 heavy chain
atgggatggagctgtatcatcctcttcttggtagcaacagctacaggcgcgcactctcaggtgcagctgcaggagtctggccctggcctggtgaagccttccgagaccctgtctctgacctgtgccgtgtccggctactccatctcctccggctactactggggctggatcagacagcctcctggcaagggcctggagtggatcggctccatctctcacaccggcaacacctactacaacccccctctgaagtccagagtgaccatctccgtggacacctccaagaaccagttctccctgaagctgtcctctgtgaccgctgccgataccgccgtgtactactgtgccagattcttgggcttcggccggccggagtactggggcaaaggcaccctggtgaccgtgtcctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 70 - protein, 467 aa, artificial
  VH5 heavy chain
MGWSCIILFLVATATGAHSQVQLQESGPGLVKPSETLSLTCAVSGYSISSGYYWGWIRQPPGKGLEWIGSISHTGNTYYNPPLKSRVTISVDTSKNQFSLKLSSVTAADTAVYYCARFLGFGRPEYWGKGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - DNA, 1401 bp, artificial
  VH6 heavy chain
atgggatggagctgtatcatcctcttcttggtagcaacagctacaggcgcgcactctcaggtgcagctgcaggagtctggccctggcctggtgaagccttccgagaccctgtctctgacctgtgccgtgtccggctactccatctcctccggctactactggggctggatcagacagcctcctggcaagggcctggagtggatcggctccatctctcacaccggcaacacctactacaacccccctctgaagtccagagtgaccatctccgtggacacctccaagaaccagttctccctgaagctgtcctctgtgaccgctgccgataccgccgtgtactactgtgccagattcttcggcttcggccgcccggagtactggggcaaaggcaccctggtgaccgtgtcctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 72 - protein, 467 aa, artificial
  VH6 heavy chain
MGWSCIILFLVATATGAHSQVQLQESGPGLVKPSETLSLTCAVSGYSISSGYYWGWIRQPPGKGLEWIGSISHTGNTYYNPPLKSRVTISVDTSKNQFSLKLSSVTAADTAVYYCARFFGFGRPEYWGKGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - DNA, 702 bp, artificial
  VL3 light chain
atgggatggagctgtatcatcctcttcttggtagcaacagctacaggcgcgcactcttcctctgagctgacccaggatcctgctgtgtctgtggccctgggccagaccgtcaggatcacctgccagggcgatagcctgagaacctactacgcctcctggtatcagcagaagcctggacaggcccctgtgctggtgatctacggcaagcacaagaggccatccggcatccctgacagattctccggctcctcctctggcaataccgcctccctgaccatcactggggctcaggcggaagacgaggctgactattactgtgtcagcagggcggtggtgggcaacccccatgttctgttcggcggagggacccagctcaccgttttaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 74 - protein, 234 aa, artificial
  VL3 light chain
MGWSCIILFLVATATGAHSSSELTQDPAVSVALGQTVRITCQGDSLRTYYASWYQQKPGQAPVLVIYGKHKRPSGIPDRFSGSSSGNTASLTITGAQAEDEADYYCVSRAVVGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 75 - DNA, 702 bp, artificial
  VL11 light chain
atgggatggagctgtatcatcctcttcttggtagcaacagctacaggcgcgcactcttcctctgagctgacccaggatcctgctgtgtctgtggccctgggccagaccgtcaggatcacctgccagggcgatagcctgagaacctactacgcctcctggtatcagcagaagcctggacaggcccctgtgctggtgatctacggcaagcacaagaggccatccggcatccctgacagattctccggctcctcctctggcaataccgcctccctgaccatcactggggctcaggcggaagacgaggctgactattactgttcctcccgctcccgccacggcaacccccatgttctgttcggcggagggacccagctcaccgttttaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 76 - protein, 234 aa, artificial
  VL11 light chain
MGWSCIILFLVATATGAHSSSELTQDPAVSVALGQTVRITCQGDSLRTYYASWYQQKPGQAPVLVIYGKHKRPSGIPDRFSGSSSGNTASLTITGAQAEDEADYYCSSRSRHGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 77 - DNA, 702 bp, artificial
  VL13 light chain
atgggatggagctgtatcatcctcttcttggtagcaacagctacaggcgcgcactcttcctctgagctgacccaggatcctgctgtgtctgtggccctgggccagaccgtcaggatcacctgccagggcgatagcctgagaacctactacgcctcctggtatcagcagaagcctggacaggcccctgtgctggtgatctacggcaagcacaagaggccatccggcatccctgacagattctccggctcctcctctggcaataccgcctccctgaccatcactggggctcaggcggaagacgaggctgactattactgtgtcacccgcaaccgctacggcaacccccatgttctgttcggcggagggacccagctcaccgttttaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 78 - protein, 234 aa, artificial
  VL13 light chain
MGWSCIILFLVATATGAHSSSELTQDPAVSVALGQTVRITCQGDSLRTYYASWYQQKPGQAPVLVIYGKHKRPSGIPDRFSGSSSGNTASLTITGAQAEDEADYYCVTRNRYGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 79 - DNA, 702 bp, artificial
  VL14 light chain
atgggatggagctgtatcatcctcttcttggtagcaacagctacaggcgcgcactcttcctctgagctgacccaggatcctgctgtgtctgtggccctgggccagaccgtcaggatcacctgccagggcgatagcctgagaacctactacgcctcctggtatcagcagaagcctggacaggcccctgtgctggtgatctacggcaagcacaagaggccatccggcatccctgacagattctccggctcctcctctggcaataccgcctccctgaccatcactggggctcaggcggaagacgaggctgactattactgtatggcgaggtcgaggaagggcaacccccatgttctgttcggcggagggacccagctcaccgttttaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 80 - protein, 234 aa, artificial
  VL14 light chain
MGWSCIILFLVATATGAHSSSELTQDPAVSVALGQTVRITCQGDSLRTYYASWYQQKPGQAPVLVIYGKHKRPSGIPDRFSGSSSGNTASLTITGAQAEDEADYYCMARSRKGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 81 - DNA, 702 bp, artificial
  VL17 light chain
atgggatggagctgtatcatcctcttcttggtagcaacagctacaggcgcgcactcttcctctgagctgacccaggatcctgctgtgtctgtggccctgggccagaccagaatcctgacctgccagggcgatagcctgagaacctactacgcctcctggtatcagcagaagcctggacaggcccctgtgctggtgatctacggcaagcacaagaggccatccggcatccctgacagattctccggctcctcctctggcaataccgcctccctgaccatcactggggctcaggcggaagacgaggctgactattactgtagcacgaggtcgaggaagggcaacccccatgttctgttcggcggagggacccagctcaccgttttaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 82 - protein, 234 aa, artificial
  VL17 light chain
MGWSCIILFLVATATGAHSSSELTQDPAVSVALGQTRILTCQGDSLRTYYASWYQQKPGQAPVLVIYGKHKRPSGIPDRFSGSSSGNTASLTITGAQAEDEADYYCSTRSRKGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 83 - DNA, 702 bp, artificial
  VL23 light chain
atgggatggagctgtatcatcctcttcttggtagcaacagctacaggcgcgcactcttcctctgagctgacccaggatcctgctgtgtctgtggccctgggccagaccgtcaggatcacctgccagggcgatagcctgagaacctactacgcctcctggtatcagcagaagcctggacaggcccctgtgctggtgatctacggcaagcacaagaggccatccggcatccctgacagattctccggctcctcctctggcaataccgcctccctgaccatcactggggctcaggcggaagacgaggctgactattactgttcggcgcggtcggtgaggggcaacccccatgttctgttcggcggagggacccagctcaccgttttaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 84 - protein, 234 aa, artificial
  VL23 light chain
MGWSCIILFLVATATGAHSSSELTQDPAVSVALGQTVRITCQGDSLRTYYASWYQQKPGQAPVLVIYGKHKRPSGIPDRFSGSSSGNTASLTITGAQAEDEADYYCSARSVRGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 85 - DNA, 702 bp, artificial
  VL24 light chain
atgggatggagctgtatcatcctcttcttggtagcaacagctacaggcgcgcactcttcctctgagctgacccaggatcctgctgtgtctgtggccctgggccagaccgtcaggatcacctgccagggcgatagcctgagaacctactacgcctcctggtatcagcagaagcctggacaggcccctgtgctggtgatctacggcaagcacaagaggccatccggcatccctgacagattctccggctcctcctctggcaataccgcctccctgaccatcactggggctcaggcggaagacgaggctgactattactgtatcgccaggagcaacaagggcaacccccatgttctgttcggcggagggacccagctcaccgttttaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 86 - protein, 234 aa, artificial
  VL24 light chain
MGWSCIILFLVATATGAHSSSELTQDPAVSVALGQTVRITCQGDSLRTYYASWYQQKPGQAPVLVIYGKHKRPSGIPDRFSGSSSGNTASLTITGAQAEDEADYYCIARSNKGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 87 - DNA, 1401 bp, artificial
  VHPTM heavy chain with signal sequence
atgggatggagctgtatcatcctcttcttggtagcaacagctacaggcgcgcactctcaggtgcagctgcaggagtctggccctggcctggtgaagccttccgagaccctgtctctgacctgtgccgtgtccggctactccatctcctccggctactactggggctggatcagacagcctcctggcaagggcctggagtggatcggctccatctctcacaccggcaacacctactacaacccccctctgaagtccagagtgaccatctccgtggacacctccaagaaccagttctccctgaagctgtcctctgtgaccgctgccgataccgccgtgtactactgtgccagaggcggcggaatctccagacctgagtactggggccagggcaccctggtgaccgtgtcctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 88 - protein, 467 aa, artificial
  VHPTM heavy chain with signal sequence
MGWSCIILFLVATATGAHSQVQLQESGPGLVKPSETLSLTCAVSGYSISSGYYWGWIRQPPGKGLEWIGSISHTGNTYYNPPLKSRVTISVDTSKNQFSLKLSSVTAADTAVYYCARGGGISRPEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - DNA, 1401 bp, artificial
  VHPDM heavy chain with signal sequence
atgggatggagctgtatcatcctcttcttggtagcaacagctacaggcgcgcactctcaggtgcagctgcaggagtctggccctggcctggtgaagccttccgagaccctgtctctgacctgtgccgtgtccggctactccatctcctccggctactactggggctggatcagacagcctcctggcaagggcctggagtggatcggctccatctctcacaccggcaacacctactacaacccccctctgaagtccagagtgaccatctccgtggacacctccaagaaccagttctccctgaagctgtcctctgtgaccgctgccgataccgccgtgtactactgtgccagaggcggcggaatctccagacctgagtactggggccagggcaccctggtgaccgtgtcctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccctgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 90 - protein, 467 aa, artificial
  VHPDM heavy chain with signal sequence
MGWSCIILFLVATATGAHSQVQLQESGPGLVKPSETLSLTCAVSGYSISSGYYWGWIRQPPGKGLEWIGSISHTGNTYYNPPLKSRVTISVDTSKNQFSLKLSSVTAADTAVYYCARGGGISRPEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - DNA, 1401 bp, artificial
  VH3TM heavy chain with signal sequence
atgggatggagctgtatcatcctcttcttggtagcaacagctacaggcgcgcactctcaggtgcagctgcaggagtctggccctggcctggtgaagccttccgagaccctgtctctgacctgtgccgtgtccggctactccatctcctccggctactactggggctggatcagacagcctcctggcaagggcctggagtggatcggctccatctctcacaccggcaacacctactacaacccccctctgaagtccagagtgaccatctccgtggacacctccaagaaccagttctccctgaagctgtcctctgtgaccgctgccgataccgccgtgtactactgtgccagattcatggggttcggccgcccggagtactggggccagggcaccctggtgaccgtgtcctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 92 - protein, 467 aa, artificial
  VH3DM heavy chain with signal sequence
MGWSCIILFLVATATGAHSQVQLQESGPGLVKPSETLSLTCAVSGYSISSGYYWGWIRQPPGKGLEWIGSISHTGNTYYNPPLKSRVTISVDTSKNQFSLKLSSVTAADTAVYYCARFMGFGRPEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - DNA, 1401 bp, artificial
  VH3DM heavy chain with signal sequence
atgggatggagctgtatcatcctcttcttggtagcaacagctacaggcgcgcactctcaggtgcagctgcaggagtctggccctggcctggtgaagccttccgagaccctgtctctgacctgtgccgtgtccggctactccatctcctccggctactactggggctggatcagacagcctcctggcaagggcctggagtggatcggctccatctctcacaccggcaacacctactacaacccccctctgaagtccagagtgaccatctccgtggacacctccaagaaccagttctccctgaagctgtcctctgtgaccgctgccgataccgccgtgtactactgtgccagattcatggggttcggccgcccggagtactggggccagggcaccctggtgaccgtgtcctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccctgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 94 - protein, 467 aa, artificial
  VH3TM heavy chain with signal sequence
MGWSCIILFLVATATGAHSQVQLQESGPGLVKPSETLSLTCAVSGYSISSGYYWGWIRQPPGKGLEWIGSISHTGNTYYNPPLKSRVTISVDTSKNQFSLKLSSVTAADTAVYYCARFMGFGRPEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - DNA, 702 bp, artificial
  VL2 light chain with signal sequence
atgggatggagctgtatcatcctcttcttggtagcaacagctacaggcgcgcactcttcctctgagctgacccaggatcctgctgtgtctgtggccctgggccagaccgtcaggatcacctgccagggcgatagcctgagaacctactacgcctcctggtatcagcagaagcctggacaggcccctgtgctggtgatctacggcaagcacaagaggccatccggcatccctgacagattctccggctcctcctctggcaataccgcctccctgaccatcactggggctcaggcggaagacgaggctgactattactgtgtcgcccggtcggtggtgggcaacccccatgttctgttcggcggagggacccagctcaccgttttaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 96 - protein, 234 aa, artificial
  VL2 light chain with signal sequence
MGWSCIILFLVATATGAHSSSELTQDPAVSVALGQTVRITCQGDSLRTYYASWYQQKPGQAPVLVIYGKHKRPSGIPDRFSGSSSGNTASLTITGAQAEDEADYYCVARSVVGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 97 - DNA, 702 bp, artificial
  VL6 light chain with signal sequence
atgggatggagctgtatcatcctcttcttggtagcaacagctacaggcgcgcactcttcctctgagctgacccaggatcctgctgtgtctgtggccctgggccagaccgtcaggatcacctgccagggcgatagcctgagaacctactacgcctcctggtatcagcagaagcctggacaggcccctgtgctggtgatctacggcaagcacaagaggccatccggcatccctgacagattctccggctcctcctctggcaataccgcctccctgaccatcactggggctcaggcggaagacgaggctgactattactgtatgagcaggagcatctggggcaacccccatgttctgttcggcggagggacccagctcaccgttttaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 98 - protein, 234 aa, artificial
  VL6 light chain with signal sequence
MGWSCIILFLVATATGAHSSSELTQDPAVSVALGQTVRITCQGDSLRTYYASWYQQKPGQAPVLVIYGKHKRPSGIPDRFSGSSSGNTASLTITGAQAEDEADYYCMSRSIWGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 99 - DNA, 702 bp, artificial
  VL9 light chain with signal sequence
atgggatggagctgtatcatcctcttcttggtagcaacagctacaggcgcgcactcttcctctgagctgacccaggatcctgctgtgtctgtggccctgggccagaccgtcaggatcacctgccagggcgatagcctgagaacctactacgcctcctggtatcagcagaagcctggacaggcccctgtgctggtgatctacggcaagcacaagaggccatccggcatccctgacagattctccggctcctcctctggcaataccgcctccctgaccatcactggggctcaggcggaagacgaggctgactattactgtgtcgccaggtccaacaagggcaacccccatgttctgttcggcggagggacccagctcaccgttttaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 100 - protein, 234 aa, artificial
  VL9 light chain with signal sequence
MGWSCIILFLVATATGAHSSSELTQDPAVSVALGQTVRITCQGDSLRTYYASWYQQKPGQAPVLVIYGKHKRPSGIPDRFSGSSSGNTASLTITGAQAEDEADYYCVARSNKGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 101 - DNA, 702 bp, artificial
  VL16 light chain  with signal sequence
atgggatggagctgtatcatcctcttcttggtagcaacagctacaggcgcgcactcttcctctgagctgacccaggatcctgctgtgtctgtggccctgggccagaccgtcaggatcacctgccagggcgatagcctgagaacctactacgcctcctggtatcagcagaagcctggacaggcccctgtgctggtgatctacggcaagcacaagaggccatccggcatccctgacagattctccggctcctcctctggcaataccgcctccctgaccatcactggggctcaggcggaagacgaggctgactattactgtgtgacgaggagcgcgaagggcaacccccatgttctgttcggcggagggacccagctcaccgttttaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 102 - protein, 234 aa, artificial
  VL16 light chain with signal sequence
MGWSCIILFLVATATGAHSSSELTQDPAVSVALGQTVRITCQGDSLRTYYASWYQQKPGQAPVLVIYGKHKRPSGIPDRFSGSSSGNTASLTITGAQAEDEADYYCVTRSAKGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 103 - DNA, 702 bp, artificial
  VL18 light chain with signal sequence
atgggatggagctgtatcatcctcttcttggtagcaacagctacaggcgcgcactcttcctctgagctgacccaggatcctgctgtgtctgtggccctgggccagaccgtcaggatcacctgccagggcgatagcctgagaacctactacgcctcctggtatcagcagaagcctggacaggcccctgtgctggtgatctacggcaagcacaagaggccatccggcatccctgacagattctccggctcctcctctggcaataccgcctccctgaccatcactggggctcaggcggaagacgaggctgactattactgtgtcacgaggagcgtgaagggcaacccccatgttctgttcggcggagggacccagctcaccgttttaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 104 - protein, 234 aa, artificial
  VL18 light chain with signal sequence
MGWSCIILFLVATATGAHSSSELTQDPAVSVALGQTVRITCQGDSLRTYYASWYQQKPGQAPVLVIYGKHKRPSGIPDRFSGSSSGNTASLTITGAQAEDEADYYCVTRSVKGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 105 - DNA, 702 bp, artificial
  VL20 light chain with signal sequence
atgggatggagctgtatcatcctcttcttggtagcaacagctacaggcgcgcactcttcctctgagctgacccaggatcctgctgtgtctgtggccctgggccagaccgtcaggatcacctgccagggcgatagcctgagaacctactacgcctcctggtatcagcagaagcctggacaggcccctgtgctggtgatctacggcaagcacaagaggccatccggcatccctgacagattctccggctcctcctctggcaataccgcctccctgaccatcactggggctcaggcggaagacgaggctgactattactgtgtctcccgcagcgcgaagggcaacccccatgttctgttcggcggagggacccagctcaccgttttaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 106 - protein, 234 aa, artificial
  VL20 light chain with signal sequence
MGWSCIILFLVATATGAHSSSELTQDPAVSVALGQTVRITCQGDSLRTYYASWYQQKPGQAPVLVIYGKHKRPSGIPDRFSGSSSGNTASLTITGAQAEDEADYYCVSRSAKGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 107 - DNA, 702 bp, artificial
  VL25 light chain with signal sequence
atgggatggagctgtatcatcctcttcttggtagcaacagctacaggcgcgcactcttcctctgagctgacccaggatcctgctgtgtctgtggccctgggccagaccgtcaggatcacctgccagggcgatagcctgagaacctactacgcctcctggtatcagcagaagcctggacaggcccctgtgctggtgatctacggcaagcacaagaggccatccggcatccctgacagattctccggctcctcctctggcaataccgcctccctgaccatcactggggctcaggcggaagacgaggctgactattactgtacgacgcggagcaacaagggcaacccccatgttctgttcggcggagggacccagctcaccgttttaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 108 - protein, 234 aa, artificial
  VL25 light chain with signal sequence
MGWSCIILFLVATATGAHSSSELTQDPAVSVALGQTVRITCQGDSLRTYYASWYQQKPGQAPVLVIYGKHKRPSGIPDRFSGSSSGNTASLTITGAQAEDEADYYCTTRSNKGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 109 - DNA, 732 bp, artificial
  H3 scFV
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgattcatggggttcggccgcccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtaactcccgggactccagtggcaacccccatgttctgttcggcggagggacccagctcaccgtttta

SEQ ID NO: 110 - protein, 244 aa, artificial
  H3 scFV
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARFMGFGRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCNSRDSSGNPHVLFGGGTQLTVL

SEQ ID NO: 111 - DNA, 732 bp, artificial
  H4 scFV
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgatggctcgggttcggccgcccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtaactcccgggactccagtggcaacccccatgttctgttcggcggagggacccagctcaccgtttta

SEQ ID NO: 112 - protein, 244 aa, artificial
  H4 scFV
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARWLGFGRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCNSRDSSGNPHVLFGGGTQLTVL

SEQ ID NO: 113 - DNA, 732 bp, artificial
  H5 scFV
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgattcttgggcttcggccggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtaactcccgggactccagtggcaacccccatgttctgttcggcggagggacccagctcaccgtttta

SEQ ID NO: 114 - protein, 244 aa, artificial
  H5 scFV
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARFLGFGRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCNSRDSSGNPHVLFGGGTQLTVL

SEQ ID NO: 115 - DNA, 732 bp, artificial
  H6 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgattcttcggcttcggccgcccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtaactcccgggactccagtggcaacccccatgttctgttcggcggagggacccagctcaccgtttta

SEQ ID NO: 116 - protein, 244 aa, artificial
  H6 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARFFGFGRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCNSRDSSGNPHVLFGGGTQLTVL

SEQ ID NO: 117 - DNA, 732 bp, artificial
  L1 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgcgtcccggtcggtgagcggcaacccccatgttctgttcggcggagggacccagctcaccgtttta

SEQ ID NO: 118 - protein, 244 aa, artificial
  L1 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCASRSVSGNPHVLFGGGTQLTVL

SEQ ID NO: 119 - DNA, 732 bp, artificial
  L2 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgtcgcccggtcggtggtgggcaacccccatgttctgttcggcggagggacccagctcaccgtttta

SEQ ID NO: 120 - protein, 244 aa, artificial
  L2 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCVARSVVGNPHVLFGGGTQLTVL

SEQ ID NO: 121 - DNA, 732 bp, artificial
  L3 scFV
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgtcagcagggcggtggtgggcaacccccatgttctgttcggcggagggacccagctcaccgtttta

SEQ ID NO: 122 - protein, 244 aa, artificial
  L3 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCVSRAVVGNPHVLFGGGTQLTVL

SEQ ID NO: 123 - DNA, 732 bp, artificial
  L4 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtagcacccgcagcagcaagggcaacccccatgttctgttcggcggagggacccagctcaccgtttta

SEQ ID NO: 124 - protein, 244 aa, artificial
  L4 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCSTRSSKGNPHVLFGGGTQLTVL

SEQ ID NO: 125 - DNA, 732 bp, artificial
  L5 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgccagcaggtcctccaagggcaacccccatgttctgttcggcggagggacccagctcaccgtttta

SEQ ID NO: 126 - protein, 244 aa, artificial
  L5 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCASRSSKGNPHVLFGGGTQLTVL

SEQ ID NO: 127 - DNA, 732 bp, artificial
  L6 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtatgagcaggagcatctggggcaacccccatgttctgttcggcggagggacccagctcaccgtttta

SEQ ID NO: 128 - protein, 244 aa, artificial
  L6 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCMSRSIWGNPHVLFGGGTQLTVL

SEQ ID NO: 129 - DNA, 732 bp, artificial
  L8 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtaccacgcgctccacccagggcaacccccatgttctgttcggcggagggacccagctcaccgtttta

SEQ ID NO: 130 - protein, 244 aa, artificial
  L8 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCTTRSTQGNPHVLFGGGTQLTVL

SEQ ID NO: 131 - DNA, 732 bp, artificial
  L9 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgtcgccaggtccaacaagggcaacccccatgttctgttcggcggagggacccagctcaccgtttta

SEQ ID NO: 132 - protein, 244 aa, artificial
  L9 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCVARSNKGNPHVLFGGGTQLTVL

SEQ ID NO: 133 - DNA, 732 bp, artificial
  L10 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtatcagccggtcgatctacggcaacccccatgttctgttcggcggagggacccagctcaccgtttta

SEQ ID NO: 134 - protein, 244 aa, artificial
  L10 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCISRSIYGNPHVLFGGGTQLTVL

SEQ ID NO: 135 - DNA, 732 bp, artificial
  L11 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgttcctcccgctcccgccacggcaacccccatgttctgttcggcggagggacccagctcaccgtttta

SEQ ID NO: 136 - protein, 244 aa, artificial
  L11 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCSSRSRHGNPHVLFGGGTQLTVL

SEQ ID NO: 137 - DNA, 732 bp, artificial
  L12 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgtcgcgagggggacgaggggcaacccccatgttctgttcggcggagggacccagctcaccgtttta

SEQ ID NO: 138 - protein, 244 aa, artificial
  L12 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCVARGTRGNPHVLFGGGTQLTVL

SEQ ID NO: 139 - DNA, 732 bp, artificial
  L13 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgtcacccgcaaccgctacggcaacccccatgttctgttcggcggagggacccagctcaccgtttta

SEQ ID NO: 140 - protein, 244 aa, artificial
  L13 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCVTRNRYGNPHVLFGGGTQLTVL

SEQ ID NO: 141 - DNA, 732 bp, artificial
  L14 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtatggcgaggtcgaggaagggcaacccccatgttctgttcggcggagggacccagctcaccgtttta

SEQ ID NO: 142 - protein, 244 aa, artificial
  L14 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCMARSRKGNPHVLFGGGTQLTVL

SEQ ID NO: 143 - DNA, 732 bp, artificial
  L15 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgttccacccgcgccatccacggcaacccccatgttctgttcggcggagggacccagctcaccgtttta

SEQ ID NO: 144 - protein, 244 aa, artificial
  L15 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCSTRAIHGNPHVLFGGGTQLTVL

SEQ ID NO: 145 - DNA, 732 bp, artificial
  L16 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgtgacgaggagcgcgaagggcaacccccatgttctgttcggcggagggacccagctcaccgtttta

SEQ ID NO: 146 - protein, 244 aa, artificial
  L16 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCVTRSAKGNPHVLFGGGTQLTVL

SEQ ID NO: 147 - DNA, 732 bp, artificial
  L17 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtagcacgaggtcgaggaagggcaacccccatgttctgttcggcggagggacccagctcaccgtttta

SEQ ID NO: 148 - protein, 244 aa, artificial
  L17 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCSTRSRKGNPHVLFGGGTQLTVL

SEQ ID NO: 149 - DNA, 732 bp, artificial
  L18 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgtcacgaggagcgtgaagggcaacccccatgttctgttcggcggagggacccagctcaccgtttta

SEQ ID NO: 150 - protein, 244 aa, artificial
  L18 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCVTRSVKGNPHVLFGGGTQLTVL

SEQ ID NO: 151 - DNA, 732 bp, artificial
  L19 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgtcgcgcgggcggtgaggggcaacccccatgttctgttcggcggagggacccagctcaccgtttta

SEQ ID NO: 152 - protein, 244 aa, artificial
  L19 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCVARAVRGNPHVLFGGGTQLTVL

SEQ ID NO: 153 - DNA, 732 bp, artificial
  L20 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgtctcccgcagcgcgaagggcaacccccatgttctgttcggcggagggacccagctcaccgtttta

SEQ ID NO: 154 - protein, 244 aa, artificial
  L20 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCVSRSAKGNPHVLFGGGTQLTVL

SEQ ID NO: 155 - DNA, 732 bp, artificial
  L21 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtgccacccgggcggtccggggcaacccccatgttctgttcggcggagggacccagctcaccgtttta

SEQ ID NO: 156 - protein, 244 aa, artificial
  L21 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCATRAVRGNPHVLFGGGTQLTVL

SEQ ID NO: 157 - DNA, 732 bp, artificial
  L23 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgttcggcgcggtcggtgaggggcaacccccatgttctgttcggcggagggacccagctcaccgtttta

SEQ ID NO: 158 - protein, 244 aa, artificial
  L23 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCSARSVRGNPHVLFGGGTQLTVL

SEQ ID NO: 159 - DNA, 732 bp, artificial
  L24 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtatcgccaggagcaacaagggcaacccccatgttctgttcggcggagggacccagctcaccgtttta

SEQ ID NO: 160 - protein, 244 aa, artificial
  L24 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCIARSNKGNPHVLFGGGTQLTVL

SEQ ID NO: 161 - DNA, 732 bp, artificial
  L25 scFv
caggtgcagctgcaggagtcgggcccaggactggtgaagacttcggagaccctgtccctcacctgcgctgtctctggttactccatcagcagtggttactactggggctggatccggcagcccccagggaaggggttggagtggattgggagtatctctcatactgggaacacctactacaacccgcccctcaagagtcgcgtcaccatatcagtagacacgtccaagaaccagttctccctgaaactgagctctgtgaccgccgcagacacggccgtgtattactgtgcgcgaggtgggggaattagcaggccggagtactggggcaaaggcaccctggtcaccgtctcgagtggaggcggcggttcaggcggaggtggctctggcggtggcggaagtgcactttcttctgagctgactcaggaccctcctgtgtctgtggccttgggacagacagtcacgctcacatgccaaggagacagcctcagaacctattatgcaagctggtaccagcagaagtcaggacaggcccctatacttctcctctatggtaaacacaaacggccctcagggatcccagaccgcttctctggctccacctcaggagacacagcttccttgaccatcactggggctcaggcggaagacgaggctgactattactgtacgacgcggagcaacaagggcaacccccatgttctgttcggcggagggacccagctcaccgtttta

SEQ ID NO: 162 - protein, 244 aa, artificial
  L25 scFv
QVQLQESGPGLVKTSETLSLTCAVSGYSISSGYYWGWIRQPPGKGLEWIGSISHTGNTYYNPPLKSRVTISVDTSKNQFSLKLSSVTAADTAVYYCARGGGISRPEYWGKGTLVTVSSGGGGSGGGGSGGGGSALSSELTQDPPVSVALGQTVTLTCQGDSLRTYYASWYQQKSGQAPILLLYGKHKRPSGIPDRFSGSTSGDTASLTITGAQAEDEADYYCTTRSNKGNPHVLFGGGTQLTVL

SEQ ID NO: 163 - protein, 6 aa, Homo sapiens
SGYYWG

SEQ ID NO: 164 - protein, 16 aa, Homo sapiens
SISHTGNTYYNPPLKS

SEQ ID NO: 165 - protein, 9 aa, artificial
  H3 heavy chain CDR3
FMGFGRPEY

SEQ ID NO: 166 - protein, 9 aa, artificial
  H4 heavy chain CDR3
WLGFGRPEY

SEQ ID NO: 167 - protein, 9 aa, artificial
  H5 heavy chain CDR3
FLGFGRPEY

SEQ ID NO: 168 - protein, 9 aa, artificial
  H6 heavy chain CDR3
FFGFGRPEY

SEQ ID NO: 169 - protein, 9 aa, Homo sapiens
GGGISRPEY

SEQ ID NO: 170 - protein, 12 aa, Homo sapiens
NSRDSSGNPHVL

SEQ ID NO: 171 - protein, 12 aa, artificial
  L1 light chain CDR3
ASRSVSGNPHVL

SEQ ID NO: 172 - protein, 12 aa, artificial
  L2 light chain CDR3
VARSVVGNPHVL

SEQ ID NO: 173 - protein, 12 aa, artificial
  L3 light chain CDR3
VSRAVVGNPHVL

SEQ ID NO: 174 - protein, 12 aa, artificial
  L4 light chain CDR3
STRSSKGNPHVL

SEQ ID NO: 175 - protein, 12 aa, artificial
  L5 light chain CDR3
ASRSSKGNPHVL

SEQ ID NO: 176 - protein, 12 aa, artificial
  L6 light chain CDR3
MSRSIWGNPHVL

SEQ ID NO: 177 - protein, 12 aa, artificial
  L8 light chain CDR3
TTRSTQGNPHVL

SEQ ID NO: 178 - protein, 12 aa, artificial
  L9 light chain CDR3
VARSNKGNPHVL

SEQ ID NO: 179 - protein, 12 aa, artificial
  L10 light chain CDR3
ISRSIYGNPHVL

SEQ ID NO: 180 - protein, 12 aa, artificial
  L11 light chain CDR3
SSRSRHGNPHVL

SEQ ID NO: 181 - protein, 12 aa, artificial
  L12 light chain CDR3
VARGTRGNPHVL

SEQ ID NO: 182 - protein, 12 aa, artificial
  L13 light chain CDR3
VTRNRYGNPHVL

SEQ ID NO: 183 - protein, 12 aa, artificial
  L14 light chain CDR3
MARSRKGNPHVL

SEQ ID NO: 184 - protein, 12 aa, artificial
  L15 light chain CDR3
STRAIHGNPHVL

SEQ ID NO: 185 - protein, 12 aa, artificial
  L16 light chain CDR3
VTRSAKGNPHVL

SEQ ID NO: 186 - protein, 12 aa, artificial
  L17 light chain CDR3
STRSRKGNPHVL

SEQ ID NO: 187 - protein, 12 aa, artificial
  L18 light chain CDR3
VTRSVKGNPHVL

SEQ ID NO: 188 - protein, 12 aa, artificial
  L19 light chain CDR3
VARAVRGNPHVL

SEQ ID NO: 189 - protein, 12 aa, artificial
  L20 light chain CDR3
VSRSAKGNPHVL

SEQ ID NO: 190 - protein, 12 aa, artificial
  L21 light chain CDR3
ATRAVRGNPHVL

SEQ ID NO: 191 - protein, 12 aa, artificial
  L23 light chain CDR3
SARSVRGNPHVL

SEQ ID NO: 192 - protein, 12 aa, artificial
  L24 light chain CDR3
IARSNKGNPHVL

SEQ ID NO: 193 - protein, 12 aa, artificial
  L25 light chain CDR3
TTRSNKGNPHVL

SEQ ID NO: 194 - protein, 11 aa, Homo sapiens
QGDSLRTYYAS

SEQ ID NO: 195 - protein, 7 aa, Homo sapiens
GKHKRPS

SEQ ID NO: 196 - protein, 330 aa, artificial
  human IgG constant region TM
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - protein, 330 aa, artificial
  human IgG constant region DM
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 199 - DNA, 100 bp, Homo sapiens
gctctgtgaccgccgcagacacggccgtgtattactgtgcgcgaggtgggggaattagcaggccggagtactggggcaaaggcaccctggtcaccgtctc

SEQ ID NO: 200 - protein, 34 aa, Homo sapiens
SSVTAADTAVYYCARGGGISRPEYWGKGTLVTVS

SEQ ID NO: 201 - DNA, 60 bp, artificial
  H3B1 VH library
cacataatgacacgcgctnnsnnsnnsnnsnnsnnsggcctcatgaccccgtttccgtgg

SEQ ID NO: 202 - protein, 20 aa, artificial
  H3B1 VH library
VYYCARGGGISRPEYWGKGT

SEQ ID NO: 203 - DNA, 56 bp, artificial
  H3B2 VH library
gacacgcgctccaccccctnnsnnsnnsnnsnnsnnsaccccgtttccgtgggacc

SEQ ID NO: 204 - protein, 18 aa, artificial
  H3B2 VH library
CARGGGISRPEYWGKGTL

SEQ ID NO: 205 - DNA, 85 bp, Homo sapiens
ggctgactattactgtaactcccgggactccagtggcaacccccatgttctgttcggcggagggacccagctcaccgttttaagt

SEQ ID NO: 206 - protein, 28 aa, Homo sapiens
ADYYCNSRDSSGNPHVLFGGGTQLTVLS

SEQ ID NO: 207 - DNA, 55 bp, artificial
  L3B1 VL library
ccgactgataatgacannsnnsnnsnnsnnsnnsccgttgggggtacaagacaag

SEQ ID NO: 208 - protein, 18 aa, artificial
  L3B1 VL library
ADYYCNSRDSSGNPHVLF

SEQ ID NO: 209 - DNA, 59 bp, artificial
  L3B2 VL library
gacattgagggccctgaggtcannsnnsnnsnnsnnsnnsaagccgcctccctgggtcg

SEQ ID NO: 210 - protein, 19 aa, artificial
  L3B2 VL library
CNSRDSSGNPHVLFGGGTQ

SEQ ID NO: 211 - DNA, 617 bp, Homo sapiens
  CDS (47)..(532)
gctgaagtgaaaacgagaccaaggtctagctctactgttggtacttatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctgaggatctaacttgcagttggacactatgttacatactctaatatagtagtgaaagtcatttctttgtattccaagtggaggag

SEQ ID NO: 212 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 213 - protein, 448 aa, artificial
  VH3DM heavy chain
QVQLQESGPGLVKPSETLSLTCAVSGYSISSGYYWGWIRQPPGKGLEWIGSISHTGNTYYNPPLKSRVTISVDTSKNQFSLKLSSVTAADTAVYYCARFMGFGRPEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 214 - protein, 215 aa, artificial
  VL2 light chain
SSELTQDPAVSVALGQTVRITCQGDSLRTYYASWYQQKPGQAPVLVIYGKHKRPSGIPDRFSGSSSGNTASLTITGAQAEDEADYYCVARSVVGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 215 - protein, 109 aa, artificial
  variable region of VL2 light chain
SSELTQDPAVSVALGQTVRITCQGDSLRTYYASWYQQKPGQAPVLVIYGKHKRPSGIPDRFSGSSSGNTASLTITGAQAEDEADYYCVARSVVGNPHVLFGGGTQLTVL

SEQ ID NO: 216 - protein, 215 aa, artificial
  VL18 light chain
SSELTQDPAVSVALGQTVRITCQGDSLRTYYASWYQQKPGQAPVLVIYGKHKRPSGIPDRFSGSSSGNTASLTITGAQAEDEADYYCVTRSVKGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 217 - protein, 109 aa, artificial
  variable region of VL18 light chain
SSELTQDPAVSVALGQTVRITCQGDSLRTYYASWYQQKPGQAPVLVIYGKHKRPSGIPDRFSGSSSGNTASLTITGAQAEDEADYYCVTRSVKGNPHVLFGGGTQLTVL

SEQ ID NO: 218 - protein, 448 aa, artificial
  VH3TM heavy chain
QVQLQESGPGLVKPSETLSLTCAVSGYSISSGYYWGWIRQPPGKGLEWIGSISHTGNTYYNPPLKSRVTISVDTSKNQFSLKLSSVTAADTAVYYCARFMGFGRPEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 448 aa, artificial
  VHPTM heavy chain
QVQLQESGPGLVKPSETLSLTCAVSGYSISSGYYWGWIRQPPGKGLEWIGSISHTGNTYYNPPLKSRVTISVDTSKNQFSLKLSSVTAADTAVYYCARGGGISRPEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - protein, 215 aa, artificial
  VL6 light chain
SSELTQDPAVSVALGQTVRITCQGDSLRTYYASWYQQKPGQAPVLVIYGKHKRPSGIPDRFSGSSSGNTASLTITGAQAEDEADYYCMSRSIWGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 221 - protein, 109 aa, artificial
  variable region of VL6 light chain
SSELTQDPAVSVALGQTVRITCQGDSLRTYYASWYQQKPGQAPVLVIYGKHKRPSGIPDRFSGSSSGNTASLTITGAQAEDEADYYCMSRSIWGNPHVLFGGGTQLTVL

SEQ ID NO: 222 - protein, 215 aa, artificial
  VL9 light chain
SSELTQDPAVSVALGQTVRITCQGDSLRTYYASWYQQKPGQAPVLVIYGKHKRPSGIPDRFSGSSSGNTASLTITGAQAEDEADYYCVARSNKGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 223 - protein, 109 aa, artificial
  variable region of VL9 light chain
SSELTQDPAVSVALGQTVRITCQGDSLRTYYASWYQQKPGQAPVLVIYGKHKRPSGIPDRFSGSSSGNTASLTITGAQAEDEADYYCVARSNKGNPHVLFGGGTQLTVL

SEQ ID NO: 224 - protein, 215 aa, artificial
  VL16 light chain
SSELTQDPAVSVALGQTVRITCQGDSLRTYYASWYQQKPGQAPVLVIYGKHKRPSGIPDRFSGSSSGNTASLTITGAQAEDEADYYCVTRSAKGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 225 - protein, 109 aa, artificial
  variable region of VL16 light chain
SSELTQDPAVSVALGQTVRITCQGDSLRTYYASWYQQKPGQAPVLVIYGKHKRPSGIPDRFSGSSSGNTASLTITGAQAEDEADYYCVTRSAKGNPHVLFGGGTQLTVL

SEQ ID NO: 226 - protein, 215 aa, artificial
  VL20 light chain
SSELTQDPAVSVALGQTVRITCQGDSLRTYYASWYQQKPGQAPVLVIYGKHKRPSGIPDRFSGSSSGNTASLTITGAQAEDEADYYCVSRSAKGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 227 - protein, 109 aa, artificial
  variable region of VL20 light chain
SSELTQDPAVSVALGQTVRITCQGDSLRTYYASWYQQKPGQAPVLVIYGKHKRPSGIPDRFSGSSSGNTASLTITGAQAEDEADYYCVSRSAKGNPHVLFGGGTQLTVL

SEQ ID NO: 228 - protein, 215 aa, artificial
  VL25 light chain
SSELTQDPAVSVALGQTVRITCQGDSLRTYYASWYQQKPGQAPVLVIYGKHKRPSGIPDRFSGSSSGNTASLTITGAQAEDEADYYCTTRSNKGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 229 - protein, 109 aa, artificial
  variable region of VL25 light chain
SSELTQDPAVSVALGQTVRITCQGDSLRTYYASWYQQKPGQAPVLVIYGKHKRPSGIPDRFSGSSSGNTASLTITGAQAEDEADYYCTTRSNKGNPHVLFGGGTQLTVL

SEQ ID NO: 230 - DNA, 34 bp, Artificial Sequence
  Primer BssHII_II_VH_F
gcttggcgcgcactctcaggtgcagctgcaggag

SEQ ID NO: 231 - DNA, 21 bp, Artificial Sequence
  Primer GVH_R_for_BssHII
tcagggagaactggttcttgg

SEQ ID NO: 232 - DNA, 21 bp, Artificial Sequence
  Primer G_VH_F_for_SalI
tccaagaaccagttctccctg

SEQ ID NO: 233 - DNA, 45 bp, Artificial Sequence
  Primer scFv_SalI_VH_R
gcgacgtcgacaggactcaccactcgagacggtgaccagggtgcc

SEQ ID NO: 234 - DNA, 32 bp, Artificial Sequence
  Primer Sal_VH_R_RJ
gcgacgtcgacaggactcaccactcgagacgg

SEQ ID NO: 235 - DNA, 34 bp, Artificial Sequence
  Primer BssHII_II_VL_F
gcttggcgcgcactcttcctctgagctgacccag

SEQ ID NO: 236 - DNA, 22 bp, Artificial Sequence
  Primer scFv_VL_R_for_BssHII
gcctgagccccagtgatggtca

SEQ ID NO: 237 - DNA, 20 bp, Artificial Sequence
  Primer GVL_F_for_XbaI
accgcctccctgaccatcac

SEQ ID NO: 238 - DNA, 50 bp, Artificial Sequence
  Primer scFv_XbaI_VL_R
gcgccgtctagagttattctactcacctaaaacggtgagctgggtccctc

SEQ ID NO: 239 - DNA, 1401 bp, artificial
  VHPTM heavy chain with signal sequence
atgggatggagctgtatcatcctcttcttggtggcaacagctacaggcgtgcactctcaggtgcagctgcaggagtctggccctggcctggtgaagccttccgagaccctgtctctgacctgtgccgtgtccggctactccatctcctccggctactactggggctggatcagacagcctcctggcaagggcctggagtggatcggctccatctctcacaccggcaacacctactacaacccccctctgaagtccagagtgaccatctccgtggacacctccaagaaccagttctccctgaagctgtcctctgtgaccgctgccgataccgccgtgtactactgtgccagaggcggcggaatctccagacctgagtactggggccagggcaccctggtgaccgtgtcctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 240 - protein, 467 aa, artificial
  VHPTM heavy chain with signal sequence
MGWSCIILFLVATATGVHSQVQLQESGPGLVKPSETLSLTCAVSGYSISSGYYWGWIRQPPGKGLEWIGSISHTGNTYYNPPLKSRVTISVDTSKNQFSLKLSSVTAADTAVYYCARGGGISRPEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 241 - DNA, 1401 bp, artificial
  VH3TM heavy chain with signal sequence
atgggatggagctgtatcatcctcttcttggtggcaacagctacaggcgtgcactctcaggtgcagctgcaggagtctggccctggcctggtgaagccttccgagaccctgtctctgacctgtgccgtgtccggctactccatctcctccggctactactggggctggatcagacagcctcctggcaagggcctggagtggatcggctccatctctcacaccggcaacacctactacaacccccctctgaagtccagagtgaccatctccgtggacacctccaagaaccagttctccctgaagctgtcctctgtgaccgctgccgataccgccgtgtactactgtgccagattcatggggttcggccgcccggagtactggggccagggcaccctggtgaccgtgtcctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 242 - protein, 467 aa, artificial
  VH3TM heavy chain with signal sequence
MGWSCIILFLVATATGVHSQVQLQESGPGLVKPSETLSLTCAVSGYSISSGYYWGWIRQPPGKGLEWIGSISHTGNTYYNPPLKSRVTISVDTSKNQFSLKLSSVTAADTAVYYCARFMGFGRPEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 243 - DNA, 703 bp, artificial
  VL2 light chain with signal sequence
atgggatggagctgtatcatcctcttcttggtggcaacagctacaggcgtgcactcttcctctgagctgacccaggatcctgctgtgtctgtggccctgggccagaccgtcaggatcacctgccagggcgatagcctgagaacctactacgcctcctggtatcagcagaagcctggacaggcccctgtgctggtgatctacggcaagcacaagaggccatccggcatccctgacagattctccggctcctcctctggcaataccgcctccctgaccatcactggggctcaggcggaagacgaggctgactattactgtgtcgcccggtcggtggtgggcaacccccatgttctgttcggcggagggacccagctcaccgttttaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcat

SEQ ID NO: 244 - protein, 234 aa, artificial
  VL2 light chain with signal sequence
MGWSCIILFLVATATGVHSSSELTQDPAVSVALGQTVRITCQGDSLRTYYASWYQQKPGQAPVLVIYGKHKRPSGIPDRFSGSSSGNTASLTITGAQAEDEADYYCVARSVVGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 245 - DNA, 703 bp, artificial
  VL18 light chain with signal sequence
atgggatggagctgtatcatcctcttcttggtggcaacagctacaggcgtgcactcttcctctgagctgacccaggatcctgctgtgtctgtggccctgggccagaccgtcaggatcacctgccagggcgatagcctgagaacctactacgcctcctggtatcagcagaagcctggacaggcccctgtgctggtgatctacggcaagcacaagaggccatccggcatccctgacagattctccggctcctcctctggcaataccgcctccctgaccatcactggggctcaggcggaagacgaggctgactattactgtgtcacgaggagcgtgaagggcaacccccatgttctgttcggcggagggacccagctcaccgttttaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcat

SEQ ID NO: 246 - protein, 234 aa, artificial
  VL18 light chain with signal sequence
MGWSCIILFLVATATGVHSSSELTQDPAVSVALGQTVRITCQGDSLRTYYASWYQQKPGQAPVLVIYGKHKRPSGIPDRFSGSSSGNTASLTITGAQAEDEADYYCVTRSVKGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 247 - DNA, 703 bp, artificial
  VL25 light chain with signal sequence
atgggatggagctgtatcatcctcttcttggtggcaacagctacaggcgtgcactcttcctctgagctgacccaggatcctgctgtgtctgtggccctgggccagaccgtcaggatcacctgccagggcgatagcctgagaacctactacgcctcctggtatcagcagaagcctggacaggcccctgtgctggtgatctacggcaagcacaagaggccatccggcatccctgacagattctccggctcctcctctggcaataccgcctccctgaccatcactggggctcaggcggaagacgaggctgactattactgtacgacgcggagcaacaagggcaacccccatgttctgttcggcggagggacccagctcaccgttttaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcat

SEQ ID NO: 248 - protein, 234 aa, artificial
  VL25 light chain with signal sequence
MGWSCIILFLVATATGVHSSSELTQDPAVSVALGQTVRITCQGDSLRTYYASWYQQKPGQAPVLVIYGKHKRPSGIPDRFSGSSSGNTASLTITGAQAEDEADYYCTTRSNKGNPHVLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2010/0075329 A1
(43) Pub. Date: Mar. 25, 2010

(54) METHODS FOR PREDICTING PRODUCTION OF ACTIVATING SIGNALS BY CROSS-LINKED BINDING PROTEINS

(76) Inventors: Margot O'Toole, Newtonville, MA (US); Yongjing Guo, Chestnut Hill, MA (US); Renee Ramsey, Charlestown, MA (US); Laird Bloom, Needham, MA (US)

Correspondence Address: FITZPATRICK CELLA (WYETH LLC), 1290 Avenue of the Americas, NEW YORK, NY, 10104-3800, US

(21) Appl. No.: 12/565,420

(22) Filed: Sep. 23, 2009

Related U.S. Application Data
(60) Provisional application No. 61/099,476, filed on Sep. 23, 2008.

(51) Int. Cl. C12Q 1/68

(52) U.S. Cl. 435/6

(57) ABSTRACT

The present invention provides human binding proteins and antigen-binding fragments thereof that specifically bind to the human interleukin-21 receptor (IL21R), and uses therefore. The invention further provides methods to predict whether the binding proteins of the invention may take on agonistic activities in vivo and produce a cytokine storm. In addition, the invention provides methods for determining whether an anti-IL21R binding protein is a neutralizing anti-IL21R binding protein, based on the identification of several IL21-responsive genes. The binding proteins can act as, e.g., antagonists of IL21R activity, thereby modulating immune responses in general, and those mediated by IL21R in particular.

### RELATED APPLICATIONS

This application claims the benefit of priority from U.S. Provisional Patent Application No. 61/099,476, filed Sep. 23, 2008, the content of which is hereby incorporated by reference herein in its entirety.

### BACKGROUND OF THE INVENTION

1. Field of the Invention

The present invention relates to methods to predict whether binding proteins can take on agonistic activities in vivo and produce a cytokine storm. These methods are useful in predicting and preventing unwanted agonistic activities produced by, for example, cross-linking of antagonistic binding proteins. Further, the studies related to the present invention focused on binding proteins and antigen-binding fragments thereof that bind interleukin-21 receptor (IL21R), in particular, human IL21R, and their use in regulating IL21R-associated activities, e.g., IL21 effects on the levels of expression of IL21-responsive genes. The binding proteins and related methods disclosed herein are useful in diagnosing and/or treating IL21R-associated disorders, e.g., inflammatory disorders, autoimmune diseases, allergies, transplant rejection, hyperproliferative disorders of the blood, and other immune system disorders.

2. Related Background Art

Antigens initiate immune responses and activate the two largest populations of lymphocytes: T cells and B cells. After encountering antigen, T cells proliferate and differentiate into effector cells, whereas B cells proliferate and differentiate into antibody-secreting plasma cells. These effector cells secrete and/or respond to cytokines, which are small proteins (less than about 30 kDa) secreted by lymphocytes and other cell types.

Human IL21 is a cytokine that shows sequence homology to IL2, IL4, and IL15 (Parrish-Novak et al. (2000) Nature 408:57-63). Despite low sequence homology among interleukin cytokines, cytokines share a common fold into a “four-helix-bundle” structure that is representative of the family. Most cytokines bind either Class I or Class II cytokine receptors. Class II cytokine receptors include the receptors for IL10 and the interferons, whereas Class I cytokine receptors include the receptors for IL2 through IL7, IL9, IL11, IL12, IL13, and IL15, as well as hematopoietic growth factors, leptin, and growth hormone (Cosman (1993) Cytokine 5:95-106).

Human IL21R is a Class I cytokine receptor. The nucleotide and amino acid sequences encoding human IL21 and its receptor (IL21R) are described in, e.g., International Application Publication Nos. WO 00/053761 and WO 01/085792; Parrish-Novak et al. (2000) supra; and Ozaki et al. (2000) Proc. Natl. Acad. Sci. USA 97:11439-44. IL21R has the highest sequence homology to the IL2 receptor β chain and the IL4 receptor α chain (Ozaki et al. (2000) supra). Upon ligand binding, IL21R associates with the common gamma cytokine receptor chain (γc) that is shared by receptor complexes for IL2, IL3, IL4, IL7, IL9, IL13, and IL15 (Ozaki et al. (2000) supra; Asao et al. (2001) J. Immunol. 167:1-5).

IL21R is expressed in lymphoid tissues, particularly on T cells, B cells, natural killer (NK) cells, dendritic cells (DC) and macrophages (Parrish-Novak et al. (2000) supra), which allows these cells to respond to IL21 (Leonard and Spolski (2005) Nat. Rev. Immunol. 5:688-98). The widespread lymphoid distribution of IL21R indicates that IL21 plays an important role in immune regulation. In vitro studies have shown that IL21 significantly modulates the function of B cells, CD4⁺ and CD8⁺T cells, and NK cells (Parrish-Novak et al. (2000) supra; Kasaian et al. (2002) Immunity 16:559-69). Recent evidence suggests that IL21-mediated signaling can have antitumor activity (Sivakumar et al. (2004) Immunology 112:177-82), and that IL21 can prevent antigen-induced asthma in mice (Shang et al. (2006) Cell. Immunol. 241:66-74).

In autoimmunity, disruption of the IL21 gene and injection of recombinant IL21 have been shown to modulate the progression of experimental autoimmune myasthenia gravis (EAMG) and experimental autoimmune encephalomyelitis (EAE), respectively (King et al. (2004) Cell 117:265-77; Ozaki et al. (2004) J. Immunol. 173:5361-71; Vollmer et al. (2005) J. Immunol. 174:2696-2701; Liu et al. (2006) J. Immunol. 176:5247-54). In these experimental systems, it has been suggested that the manipulation of IL21-mediated signaling directly altered the function of CD8⁺ cells, B cells, T helper cells, and NK cells. Thus, manipulation of the IL21-mediated signaling pathway may be an effective way to diagnose, prevent, treat, or ameliorate IL21-associated disorders, such as inflammatory disorders (e.g., lung inflammation (e.g., pleurisy), chronic obstructive pulmonary disease (COPD)), autoimmune diseases, allergies, transplant rejection, hyperproliferative disorders of the blood, and other immune system disorders. As such, IL21R antagonists, e.g., anti-IL21R binding proteins, can serve as therapeutic agents for treating IL21-associated disorders.

As the general therapeutic objective of anti-IL21R therapy is inhibition of IL21-mediated immune activation, it is critical to demonstrate that anti-IL21R binding proteins do not deliver an activation (or agonistic) signal, even when cross-linked. Concern regarding the agonistic potential of cross-linked therapeutic binding proteins has been heightened by the life-threatening immunotoxic cytokine storm response to intravenous administration of an anti-CD28 antibody, TGN1412 (Suntharalingham et al. (2006) N. Engl. J. Med. 355:1018-28). This cytokine storm response, a type of proinflammatory cascade, was observed within hours of treatment in six healthy male adults. The hypothesis in the case of TGN1412 was that the antibodies became cross-linked in vivo and induced the cytokine storm response in the human subjects. Experiments performed after the clinical study demonstrated that a profound in vitro agonistic signal was delivered by cross-linked TGN1412, but not soluble TGN1412 (Stebbings et al. (2007) J. Immunol. 179(5):3325-31). In light of the TGN1412 experience, concern exists that binding proteins, e.g., antibodies, particularly those directed against receptors on immune system cells, may take on agonistic activities in vivo. Therefore, it is of critical importance to determine whether activation signals can be delivered by cross-linked anti-IL21R binding proteins.

### SUMMARY OF THE INVENTION

The present invention provides methods to predict whether the binding proteins of the invention may take on agonistic activities in vivo and produce a cytokine storm or other form of proinflammatory cascade. In addition, the invention provides methods for determining whether an anti-IL21R binding protein is a neutralizing anti-IL21R binding protein, based on the identification of several IL21-responsive genes. The invention provides several other methods related to, at least in part, the identification of sets of genes related to cytokine storm and/or IL21 responsiveness. In addition, methods of predicting whether a therapeutic binding protein will induce an activation signal mediated through IL21R by determining whether in vitro cross-linked binding proteins induce gene activation of any gene activated by IL21 (i.e., IL21-responsive genes) are provided. The binding proteins described herein are derived from antibody 18A5, which is disclosed in U.S. Pat. No. 7,495,085, the entirety of which is hereby incorporated by reference herein. The binding proteins disclosed herein have a much greater degree of affinity to human and/or murine IL-21R than does the parental 18A5 antibody

In at least one embodiment, the present invention provides a method of predicting whether a therapeutic binding protein will induce a cytokine storm upon administration to a first mammalian subject comprising the steps of: administering the therapeutic binding protein to a second mammalian subject, wherein the second mammalian subject is a binding protein-treated second mammalian subject; obtaining a blood sample from the binding protein-treated second mammalian subject; determining the level of expression of at least one cytokine storm gene in the blood of the binding protein-treated second mammalian subject; and comparing the level of expression of the at least one cytokine storm gene in the blood of the binding protein-treated second mammalian subject to the level of expression of the at least one cytokine storm gene in the blood of an untreated second mammalian subject, wherein a level of expression of the at least one cytokine storm gene in the binding protein-treated second mammalian subject substantially greater than the level of expression of the at least one cytokine storm gene in an untreated second mammalian subject indicates that the therapeutic binding protein will induce a cytokine storm in the first mammalian subject. In some embodiments, the first mammalian subject is a human subject. In some embodiments, the therapeutic binding protein is an anti-IL21R binding protein (e.g., AbA-AbZ). In certain embodiments, the second mammalian subject is a member of a safety study species (e.g., a cynomolgus monkey subject). In some embodiments, the at least one cytokine storm gene is selected from the group consisting of: IL4, IL2, IL1β, IL12, TNF, IFNγ, IL6, IL8, and IL10. The method can comprise determining the levels of expression or at least two, at least three, at least four, at least five, at least six, at least seven, at least eight, or at least nine or more cytokine storm genes. In some embodiments, the method of determining the level of expression of at least one cytokine storm gene in the blood of the binding protein-treated second mammalian subject comprises measuring the level of mRNA expression of the at least one cytokine storm gene. In some embodiments, the determining comprises measuring the level of protein expression of the at least one cytokine storm gene (for example, measuring the level of cytokine release of the at least one cytokine storm gene).

In at least one embodiment, the invention provides a method of predicting whether a therapeutic binding protein will induce a cytokine storm in a mammalian subject comprising the steps of: obtaining a blood sample from the mammalian subject; incubating the therapeutic binding protein with the blood sample, wherein the blood sample is a binding protein-treated blood sample; determining the level of expression of at least one cytokine storm gene in the binding protein-treated blood sample; and comparing the level of expression of the at least one cytokine storm gene in the binding protein-treated blood sample to the level of expression of the at least one cytokine storm gene in an untreated or a negative control-treated blood sample, wherein a level of expression of the at least one cytokine storm gene in the binding protein-treated blood sample substantially greater than the level of expression of the at least one cytokine storm gene in the untreated or negative control-treated blood sample indicates that the therapeutic binding protein will induce a cytokine storm in the mammalian subject. In some embodiments, a level of expression of the at least one cytokine storm gene in the binding protein-treated blood sample substantially less than the level of expression of the at least one cytokine storm gene in the untreated or negative control-treated blood sample indicates that the therapeutic binding protein will not induce a cytokine storm in the mammalian subject. In some embodiments, the mammalian subject is a human subject. In some embodiments, the mammalian subject is a member of a safety study species (e.g., a cynomolgus monkey subject). In some embodiments of the invention, the blood sample is a purified peripheral blood mononuclear cell (PBMC) sample. In further embodiments, the therapeutic binding protein is an anti-IL21R binding protein; the at least one cytokine storm gene is selected from the group consisting of: IL4, IL2, IL1β, IL12, TNF, IFNγ, IL6, IL8, and IL10; and the method comprises determining the levels of expression or at least two, at least three, at least four, at least five, at least six, at least seven, at least eight, or at least nine cytokine storm genes. In some embodiments, the method of determining the level of expression of at least one cytokine storm gene in the binding protein-treated blood sample comprises measuring the level of mRNA expression of the at least one cytokine storm gene. In some other embodiments, the determining comprises measuring the level of protein expression of the at least one cytokine storm gene (for example, measuring the level of cytokine release of the at least one cytokine storm gene).

In at least one embodiment, the present invention provides a method of determining whether an anti-IL21R binding protein is a neutralizing anti-IL21R binding protein comprising the steps of: contacting a first blood sample from a subject with an IL21 ligand; determining a level of expression of at least one IL21-responsive gene in the first blood sample contacted with the IL21 ligand; contacting a second blood sample from the subject with the IL21 ligand in the presence of an anti-IL21R binding protein; determining the level of expression of the at least one IL21-responsive gene in the second blood sample contacted with the IL21 ligand in the presence of the anti-IL21R binding protein; and comparing the determined levels of expression of the at least one IL21-responsive gene, wherein a change in the level of expression of the at least one IL21-responsive gene indicates that the anti-IL21R binding protein is a neutralizing binding protein. In some embodiments, the subject is a mammal (e.g., human, monkey, a member of a safety study species). In some embodiments, the at least one IL21-responsive gene is selected from the group consisting of CCL19, CCL2, CCL3, CCR2, CD19, CD40, CSF2, CSF3, CXCL10, CXCL11, GZMB, IFNγ, IL10, IL12β, IL1β, IL2RA, IL6, PRF1, PTGS2, and TBX21.

The invention also provides a method of determining whether an anti-IL21R binding protein is a therapeutic anti-IL21R binding protein comprising the steps of: contacting a first blood sample from a subject with an IL21 ligand; determining a level of expression of at least one IL21-responsive gene in the first blood sample contacted with the IL21 ligand; contacting a second blood sample from the subject with the IL21 ligand in the presence of an anti-IL21R binding protein; determining the level of expression of the at least one IL21-responsive gene in the second blood sample contacted with the IL21 ligand in the presence of the anti-IL21R binding protein; and comparing the two levels of expression of the at least one IL21-responsive gene, wherein a substantial change in the level of expression of the at least one IL21-responsive gene indicates that the anti-IL21R binding protein is a therapeutic binding protein. In some embodiments, the subject is a mammal (e.g., human, monkey, a member of a safety study species). In some embodiments, the at least one IL21-responsive gene is selected from the group consisting of CCL19, CCL2, CCL3, CCR2, CD19, CD40, CSF2, CSF3, CXCL10, CXCL11, GZMB, IFNγ, IL10, IL12β, IL1β, IL2RA, IL6, PRF1, PTGS2, and TBX21.

The present invention also provides a method of determining the pharmacodynamic activity of an anti-IL21R binding protein comprising detecting a modulation in a level of expression of at least one IL21-responsive gene in a blood sample of a subject. In at least one embodiment of this method, detecting the modulation in the level of expression of the at least one IL21-responsive gene comprises the steps of: administering the anti-IL21R binding protein to the subject, wherein the subject is treated with the anti-IL21R binding protein; contacting a blood sample from the subject treated with the anti-IL21R binding protein with an IL21 ligand; determining the level of expression of the at least one IL21-responsive gene in the blood sample from the subject treated with the anti-IL21R binding protein and contacted with the IL21 ligand; and comparing the determined level of expression of the at least one IL21-responsive gene with the level of expression of the at least one IL21-responsive gene in a blood sample contacted with the IL21 ligand, wherein the blood sample is from a subject not treated with the anti-IL21R binding protein. In some embodiments, the subject is a mammal (e.g., monkey, human). In some embodiments, the at least one IL21-responsive gene is selected from the group consisting of CCL19, CCL2, CCL3, CCR2, CD19, CD40, CSF2, CSF3, CXCL10, CXCL11, GZMB, IFNγ, IL10, IL12β, IL1β, IL2RA, IL6, PRF1, PTGS2, and TBX21. In some further embodiments, the at least one IL21-responsive gene is selected from CD19, GZMB, PRF1, IL2RA, IFNγ, and IL6.

The present invention also provides a method of diagnosing a test subject with an IL21R-associated disorder comprising detecting a difference in a level of expression of at least one IL21-responsive gene in an immune cell of a blood sample of the test subject compared with a healthy subject. In at least one embodiment, the method comprises the steps of: determining the level of expression of the at least one IL21-responsive gene in a blood sample from a healthy subject; determining the level of expression of the at least one IL21-responsive gene in a blood sample from a test subject; and comparing the expression levels of the at least one IL21-responsive gene, wherein a difference in the level of expression of the at least one IL21-responsive gene indicates that the test subject is afflicted with an IL21R-associated disorder. In some embodiments, the subject is a mammal (e.g., monkey, human). In some embodiments, the at least one IL21-responsive gene is selected from the group consisting of CCL19, CCL2, CCL3, CCR2, CD19, CD40, CSF2, CSF3, CXCL10, CXCL11, GZMB, IFNγ, IL10, IL12β, IL1β, IL2RA, IL6, PRF1, PTGS2, and TBX21. In some further embodiments, the at least one IL21-responsive gene is selected from CD19, GZMB, PRF1, IL2RA, IFNγ, and IL6. In some embodiments, the IL21R-associated disorder is selected from the group consisting of an autoimmune disorder, an inflammatory condition, an allergy, a transplant rejection, and a hyperproliferative disorder of the blood.

The present invention also provides a method of predicting whether a therapeutic binding protein will induce an activation signal mediated through IL21R by determining whether in vitro cross-linked binding protein induces gene activation of any gene activated by IL21 (i.e., IL21-responsive genes).

Additional aspects of the disclosure will be set forth in part in the description, and in part will be obvious from the description, or may be learned by practicing the invention. The invention is set forth and particularly pointed out in the claims, and the disclosure should not be construed as limiting the scope of the claims.

The following detailed description includes exemplary representations of various embodiments of the invention, which are not restrictive of the invention as claimed. The accompanying figures constitute a part of this specification and, together with the description, serve only to illustrate embodiments and not limit the invention.

### BRIEF DESCRIPTION OF THE DRAWINGS

FIG. 1A demonstrates relative quantification (RQ; Y-axis) of gene expression of six examined genes (CD19, GZMB, IFNγ (IFNG), IL2RA, IL6, and PRF1) at different concentrations of IL21 at either 2, 4, 6, or 24 hr time points (X-axis). FIG. 1B depicts percent inhibition (Y-axis) of IL21 response of the same genes after treatment with different concentrations of AbS (X-axis).

FIG. 2 depicts either in vitro protein (FIG. 2A) or in vitro RNA (FIG. 2B) signal induced by IL21. FIG. 2A shows the magnitude of either TNF or IL8 protein signal (Y-axis; stimulated/control) in peripheral blood mononuclear cells (PBMCs) from five individual human donors after treatment with 33 ng/mL IL21 (X-axis), as compared to the reported response after treatment with 1 μg/well TGN1412. FIG. 2B depicts the effects of either anti-CD28 antibody or AbS (represented in comparison to IgGTM control) (Y-axis; average log₂ fold-change) on gene activation of various gene transcripts (X-axis).

FIG. 3 depicts a scheme for testing binding protein—(e.g., anti-IL21R antibody)—mediated PBMC activation in vitro.

FIG. 4 depicts results from a confirmatory ELISA demonstrating persistence of several coated antibodies at indicated concentrations (X-axis) in both dry and anti-IgG-coated plates, as measured by O.D. at 450 nm (Y-axis).

FIG. 5 depicts the procedure used for an in vitro test of cross-linked AbS on PBMCs from human donors to determine upregulation of RNA expression or cytokine release in response to AbS.

FIG. 6 depicts the effects of cross-linked AbS on cytokine release and RNA expression in in vitro experiments on PBMCs from five individual human donors.

FIG. 6A represents the effects of cross-linked AbS, IL21 (positive control), and IgGTM, IgG1, and IgGFc (all negative controls) (X-axis) at indicated concentrations on induction of IFNγ release (expressed as change relative to media control; pg/ml; Y-axis) at a 20-hr time point. FIG. 6B represents the effects of AbS or IL21 at indicated concentrations on expression of various indicated RNAs (Y-axis; fold-change relative to IgGTM control), at a 4-hr time point, with the experiments performed either in dry-coated plates or on anti-IgG coated plates.

FIG. 7 depicts the effects of IL21 stimulation on IL2RA and TNFα responses in cynomolgus monkey blood (Y-axis; increase in RNA concentration over unstimulated blood) as compared with the effect of LPS- or PHA-stimulation.

FIG. 8 depicts the effects of AbS at three indicated concentrations on IL21-stimulated IL2RA expression (Y-axis; relative IL2RA expression level (RQ)) as compared to IgG control, in an ex vivo experiment on cynomolgus monkey blood.

FIG. 9 depicts the effects of AbS on TNFα and IFNγ (Y-axis; change in RNA concentration relative to baseline (where baseline is set as 1)) at different time points in an in vivo experiment on AbS-treated cynomolgus monkeys, as compared to untreated monkeys. The results are also compared to the effects of LPS- or PHA-stimulation on TNF in a 2-hr in vitro experiment (inset); A and B represent experiments with whole cell blood from two different cynomolgus monkeys.

### DETAILED DESCRIPTION OF THE INVENTION

The anti-IL21R binding proteins disclosed herein have been described as potent inhibitors of IL21 activity, and represent promising therapeutic agents for treating IL21-associated disorders. The properties of anti-IL21R binding proteins, including but not limited to their pharmacokinetic and pharmacodynamic activities, are described in detail in U.S. patent application Ser. No. 12/472,237, filed May 26, 2009, and U.S. Provisional Patent Application No. 61/055,543, filed May 23, 2008, both of which are incorporated by reference herein in their entireties.

Specifically, several binding proteins, e.g., several within the range of AbA-AbZ as disclosed herein, including AbS, potently block IL21 interaction with IL21R, thereby modulating expression of IL21-responsive cytokines or genes, without inducing the IL21 pathway or cytokine storm. Determining whether a protein antagonist, such as an antagonistic binding protein, induces an adverse immune reaction upon administration, such as inducing a cytokine storm, is now understood to be an important step in the development and testing of a new therapeutic agent and/or in evaluating the safety profile of a potential therapeutic product prior to, during, and/or after approval of the product by a regulatory agency (e.g., the U.S. Food and Drug Administration). Thus, the present invention utilizes a novel assay to test the effects of binding proteins, e.g., antibodies, e.g., antagonistic anti-IL21R antibodies, on cytokine storm induction. As a result, AbS and other binding proteins are demonstrated herein to be potent inhibitors of the IL21 pathway that do not induce cytokine storm activation; thus, these binding proteins represent promising therapeutic targets.

### DEFINITIONS

In order that the present invention may be more readily understood, certain terms are first defined. Additional definitions are set forth throughout the detailed description and elsewhere in the specification.

The terms “interleukin-21 receptor” or “IL21R” or the like refer to a Class I cytokine family receptor, also known as MU-1 (see, e.g., U.S. patent application Ser. No. 09/569,384 and U.S. Patent Application Publication Nos. 2004/0265960; 2006/0159655; 2006/0024268; and 2008/0241098), NILR or zalphal1 (see, e.g., International Application Publication No. WO 01/085792; Parrish-Novak et al. (2000) supra; Ozaki et al. (2000) supra), that binds to an IL21 ligand. IL21R is homologous to the shared β chain of the IL2 and IL15 receptors, and IL4α (Ozaki et al. (2000) supra). Upon ligand binding, IL21R is capable of interacting with a common gamma cytokine receptor chain (γc) and inducing the phosphorylation of STAT1 and STAT3 (Asao et al. (2001) supra) or STAT5 (Ozaki et al. (2000) supra). IL21R shows widespread lymphoid tissue distribution. The terms “interleukin-21 receptor” or “IL21R” or the like also refer to a polypeptide (preferably of mammalian origin, e.g., murine or human IL21R) or, as context requires, a polynucleotide encoding such a polypeptide, that is capable of interacting with IL21 (preferably IL21 of mammalian origin, e.g., murine or human IL21) and has at least one of the following features: (1) an amino acid sequence of a naturally occurring mammalian IL21R polypeptide or a fragment thereof, e.g., an amino acid sequence set forth in SEQ ID NO:2 (human-corresponding to GENBANK® (U.S. Dept. of Health and Human Services, Bethesda, Md.) Accession No. NP_{—}068570) or SEQ ID NO:4 (murine—corresponding to GENBANK® Acc. No. NP_{—}068687), or a fragment thereof; (2) an amino acid sequence substantially homologous to, e.g., at least 85%, 90%, 95%, 98%, or 99% homologous to, an amino acid sequence set forth in SEQ ID NO:2 or SEQ ID NO:4, or a fragment thereof; (3) an amino acid sequence that is encoded by a naturally occurring mammalian IL21R nucleotide sequence or fragment thereof (e.g., SEQ ID NO:1 (human—corresponding to GENBANK® Accession No. NM_{—}021798) or SEQ ID NO:3 (murine—corresponding to GENBANK® Acc. No. NM_{—}021887), or a fragment thereof); (4) an amino acid sequence encoded by a nucleotide sequence that is substantially homologous to, e.g., at least 85%, 90%, 95%, 98%, or 99% homologous to, a nucleotide sequence set forth in SEQ ID NO:1 or SEQ ID NO:3 or a fragment thereof; (5) an amino acid sequence encoded by a nucleotide sequence degenerate to a naturally occurring IL21R nucleotide sequence or a fragment thereof, e.g., SEQ ID NO:1 or SEQ ID NO:3, or a fragment thereof; or (6) a nucleotide sequence that hybridizes to one of the foregoing nucleotide sequences under stringent conditions, e.g., highly stringent conditions. In addition, other nonhuman and nonmammalian IL21Rs are contemplated as useful in the disclosed methods.

The term “interleukin-21” or “IL21” refers to a cytokine that shows sequence homology to IL2, IL4 and IL15 (Parrish-Novak et al. (2000) supra), and binds to an IL21R. Such cytokines share a common fold into a “four-helix-bundle” structure that is representative of the family. IL21 is expressed primarily in activated CD4⁺T cells, and has been reported to have effects on NK, B and T cells (Parrish-Novak et al. (2000) supra; Kasaian et al. (2002) supra). Upon IL21 binding to IL21R, activation of IL21R leads to, e.g., STAT5 or STAT3 signaling (Ozaki et al. (2000) supra). The term “interleukin-21” or “IL21” also refers to a polypeptide (preferably of mammalian origin, e.g., murine or human IL21), or as context requires, a polynucleotide encoding such a polypeptide, that is capable of interacting with IL21R (preferably of mammalian origin, e.g., murine or human IL21R) and has at least one of the following features: (1) an amino acid sequence of a naturally occurring mammalian IL21 or a fragment thereof, e.g., an amino acid sequence set forth in SEQ ID NO:212 (human), or a fragment thereof; (2) an amino acid sequence substantially homologous to, e.g., at least 85%, 90%, 95%, 98%, or 99% homologous to, an amino acid sequence set forth in SEQ ID NO:212, or a fragment thereof; (3) an amino acid sequence that is encoded by a naturally occurring mammalian IL21 nucleotide sequence or a fragment thereof (e.g., SEQ ID NO:211 (human), or a fragment thereof); (4) an amino acid sequence encoded by a nucleotide sequence that is substantially homologous to, e.g., at least 85%, 90%, 95%, 98%, or 99% homologous to, a nucleotide sequence set forth in SEQ ID NO:211 or a fragment thereof; (5) an amino acid sequence encoded by a nucleotide sequence degenerate to a naturally occurring IL21 nucleotide sequence or a fragment thereof; or (6) a nucleotide sequence that hybridizes to one of the foregoing nucleotide sequences under stringent conditions, e.g., highly stringent conditions.

The terms “IL21R activity” and the like (e.g., “activity of IL21R,” “IL21/IL21R activity”) refer to at least one cellular process initiated or interrupted as a result of IL21R binding. IL21R activities include, but are not limited to: (1) interacting with, e.g., binding to, a ligand, e.g., an IL21 polypeptide; (2) associating with or activating signal transduction (also called “signaling,” which refers to the intracellular cascade occurring in response to a particular stimuli) and signal transduction molecules (e.g., gamma chain (γc) and JAK1), and/or stimulating the phosphorylation and/or activation of STAT proteins, e.g., STAT5 and/or STAT3; (3) modulating the proliferation, differentiation, effector cell function, cytolytic activity, cytokine secretion, and/or survival of immune cells, e.g., T cells, NK cells, B cells, macrophages, regulatory T cells (Tregs) and megakaryocytes; and (4) modulating expression of IL21-responsive genes or cytokines, e.g., modulating IL21 effects on the level of expression of, e.g., CCL19, CCL2, CCL3, CCR2, CD19, CD40, CSF2, CSF3, CXCL10, CXCL11, GZMB, IFNγ, IL10, IL12β, IL1β, IL2RA, IL6, PRF1, PTGS2, and TBX21.

The term “binding protein” as used herein includes any naturally occurring, recombinant, synthetic, or genetically engineered protein, or a combination thereof, that binds an antigen, target protein, or peptide, or a fragment(s) thereof. Binding proteins related to the present invention can include antibodies, or can be derived from at least one antibody fragment. The binding proteins can include naturally occurring proteins and/or proteins that are synthetically engineered. Binding proteins of the invention can bind to an antigen or a fragment thereof to form a complex and elicit a biological response (e.g., agonize or antagonize a particular biological activity). Binding proteins can include isolated antibody fragments, “Fv” fragments consisting of the variable regions of the heavy and light chains of an antibody, recombinant single-chain polypeptide molecules in which light and heavy chain variable regions are connected by a peptide linker (“scFv proteins”), and minimal recognition units consisting of the amino acid residues that mimic the hypervariable region. Binding protein fragments can also include functional fragments of an antibody, such as, for example, Fab, Fab′, F(ab′)₂, Fc, Fd, Fd′, Fv, and a single variable domain of an antibody (dAb). The binding proteins can be double or single chain, and can comprise a single binding domain or multiple binding domains.

The term “antibody” as used herein refers to an immunoglobulin that is reactive to a designated protein or peptide or fragment thereof. Suitable antibodies include, but are not limited to, human antibodies, primatized antibodies, chimeric antibodies, monoclonal antibodies, monospecific antibodies, polyclonal antibodies, polyspecific antibodies, nonspecific antibodies, bispecific antibodies, multispecific antibodies, humanized antibodies, synthetic antibodies, recombinant antibodies, hybrid antibodies, mutated antibodies, grafted conjugated antibodies (i.e., antibodies conjugated or fused to other proteins, radiolabels, cytotoxins), and in vitro-generated antibodies. The antibodies of the invention can be derived from any species including, but not limited to mouse, rat, human, camel, llama, fish, shark, goat, rabbit, chicken, and bovine. Typically, the antibody specifically binds to a predetermined antigen, e.g., an antigen (e.g., IL21R) associated with a disorder, e.g., an inflammatory, immune, autoimmune, neurodegenerative, metabolic, and/or malignant disorder.

Binding proteins comprising antibodies (immunoglobulins) are typically tetrameric glycosylated proteins composed of two light (L) chains of approximately 25 kDa each and two heavy (H) chains of approximately 50 kDa each. Two types of light chains, termed lambda (λ) and kappa (κ), may be found in antibodies. Depending on the amino acid sequence of the constant domain of heavy chains, immunoglobulins can be assigned to five major classes: A, D, E, G, and M (i.e., IgA, IgD, IgE, IgG, and IgM), and several of these may be further divided into subclasses (isotypes), e.g., IgG1, IgG2, IgG3, IgG4, IgA1, and IgA2. Each light chain includes an N-terminal variable (V) domain (V_{L}) and a constant (C) domain (C_{L}). Each heavy chain includes an N-terminal V domain (V_{H}), three or four C domains (C_{H}s), and a hinge region. The C_{H} domain most proximal to V_{H} is designated as C_{H}1. The V_{H} and V_{L} domains consist of four regions of relatively conserved sequences called framework regions (FR1, FR2, FR3, and FR4) that form a scaffold for three regions of hypervariable sequences, called CDRs. The CDRs contain most of the residues responsible for specific interactions of the antibody with the antigen. CDRs are referred to as CDR1, CDR2, and CDR3. CDR constituents on the heavy chain are referred to as H1, H2, and H3 (also referred to herein as CDR H1, CDR H2, and CDR H3, respectively), while CDR constituents on the light chain are referred to as L1, L2, and L3 (also referred to herein as CDR L1, CDR L2, and CDR L3, respectively).

CDR3 is typically the greatest source of molecular diversity within the antigen-binding site. CDR H3, for example, can be as short as two amino acid residues or greater than 26 amino acids. The subunit structures and three-dimensional configurations of different classes of immunoglobulins are well known in the art. For a review of antibody structure, see, e.g., Antibodies: A Laboratory Manual, eds. Harlow et al., Cold Spring Harbor Laboratory (1988). One of skill in the art will recognize that each subunit structure, e.g., a C_{H}, V_{H}, C_{L}, V_{L}, CDR, and/or FR structure, comprises active fragments, e.g., the portion of the V_{H}, V_{L}, or CDR subunit that binds to the antigen, i.e., the antigen-binding fragment, or, e.g., the portion of the C_{H} subunit that binds to and/or activates, e.g., an Fc receptor and/or complement. The CDRs typically refer to the Kabat CDRs (as described in Kabat et al. (5th ed. 1991) Sequences of Proteins of Immunological Interest, U.S. Department of Health and Human Services, NIH Publication No. 91-3242). Another standard for characterizing the antigen binding site is to refer to the hypervariable loops as described in, e.g., Chothia et al. (1992) J. Mol. Biol. 227:799-817 and Tomlinson et al. (1995) EMBO J. 14:4628-38. Still another standard is the “AbM” definition used by Oxford Molecular's AbM antibody modeling software (see, generally, e.g., Protein Sequence and Structure Analysis of Antibody Variable Domains in: Antibody Engineering (2001) eds. Kontermann and Dübel, Springer-Verlag, Heidelberg). Embodiments described with respect to Kabat CDRs can alternatively be implemented using similar described relationships with respect to Chothia hypervariable loops or to the AbM-defined loops.

The sequence of antibody genes after assembly and somatic mutation is highly varied, and these varied genes are estimated to encode 10¹⁰ different antibody molecules (Immunoglobulin Genes, 2nd ed. (1995) eds. Jonio et al., Academic Press, San Diego, Calif.).

The terms “antigen-binding domain” and “antigen-binding fragment” refer to a part of a binding protein (i.e., a binding protein fragment) that comprises amino acids responsible for the specific binding between the binding protein and an antigen. The part of the antigen that is specifically recognized and bound by the binding protein is referred to as the “epitope.” An antigen-binding domain may comprise a light chain variable region (V_{L}) and a heavy chain variable region (V_{H}) of an antibody; however, it does not have to comprise both. Fd fragments, for example, have two V_{H} regions and often retain antigen-binding function of the intact antigen-binding domain. Examples of antigen-binding fragments of a binding protein include, but are not limited to: (1) a Fab fragment, a monovalent fragment having V_{L}, V_{H}, C_{L} and C_{H}1 domains; (2) a F(ab′)₂ fragment, a bivalent fragment having two Fab fragments linked by a disulfide bridge at the hinge region; (3) an Fd fragment, having two V_{H} and one C_{H}1 domains; (4) an Fv fragment, having the V_{L} and V_{H} domains of a single arm of an antibody; (5) a dAb fragment (see, e.g., Ward et al. (1989) Nature 341:544-46), having a V_{H} domain; (6) an isolated CDR; and (7) a single chain variable fragment (scFv). The Fab fragment consists of V_{H}-C_{H}1 and V_{L}-C_{L} domains covalently linked by a disulfide bond between the constant regions. The Fv fragment is smaller and consists of V_{H} and V_{L} domains noncovalently linked. Although the two domains of an Fv fragment, V_{L} and V_{H} are coded for by separate genes, they can be joined, using recombinant methods, by a synthetic linker that enables them to be made as a single protein chain in which the V_{L} and V_{H} regions pair to form monovalent molecules (known as scFv) (see, e.g., Bird et al. (1988) Science 242:423-26; Huston et al. (1988) Proc. Natl. Acad. Sci. USA 85:5879-83). This is done to overcome the tendency of noncovalently linked domains to dissociate. The synthetic polypeptide linker links (1) the C-terminus of V_{H} to the N-terminus of V_{L}, or (2) the C-terminus of V_{L} to the N-terminus of V_{H}. A 15-mer (Gly₄Ser)₃ peptide, for example, may be used as a linker, but other linkers are known in the art. The antigen-binding fragments can be obtained using conventional techniques known to those with skill in the art, and the fragments are evaluated for function in the same manner as are intact binding proteins such as, for example, antibodies.

Numerous methods known to those skilled in the art are available for obtaining binding proteins or antigen-binding fragments thereof. For example, anti-IL21R binding proteins, including anti-IL21R antibodies, can be produced using recombinant DNA methods (see, e.g., U.S. Pat. No. 4,816,567). Monoclonal antibodies may also be produced by generation of hybridomas in accordance with known methods (see, e.g., Kohler and Milstein (1975) Nature, 256:495-99). Hybridomas formed in this manner are then screened using standard methods, such as enzyme-linked immunosorbent assays (ELISA) and surface plasmon resonance (BIACORE™) analysis, to identify one or more hybridomas that produce an antibody that specifically binds with a particular antigen. Any form of the specified antigen may be used as the immunogen, e.g., recombinant antigen, naturally occurring forms, any variants or fragments thereof, and antigenic peptides thereof.

One exemplary method of making antibodies includes screening protein expression libraries, e.g., phage or ribosome display libraries. Phage display is described, for example, in U.S. Pat. No. 5,223,409; Smith (1985) Science 228:1315-17; Clackson et al. (1991) Nature 352:624-28; Marks et al. (1991) J. Mol. Biol. 222:581-97; WO 92/018619; WO 91/017271; WO 92/020791; WO 92/015679; WO 93/001288; WO 92/001047; WO 92/009690; and WO 90/002809. As described in detail in U.S. application Ser. No. 12/472,237, some antibodies related to the present invention were produced by phage display techniques.

In addition to the use of display libraries, the specified antigen can be used to immunize a nonhuman animal, e.g., monkey, chicken, and rodent (e.g., mouse, hamster, and rat). In one embodiment, the nonhuman animal includes at least a part of a human immunoglobulin gene. For example, it is possible to engineer mouse strains deficient in mouse antibody production with large fragments of the human Ig loci. Using the hybridoma technology, antigen-specific monoclonal binding proteins derived from the genes with the desired specificity may be produced and selected (see, e.g., XENOMOUSE™, Green et al. (1994) Nat. Genet. 7:13-21, U.S. Pat. No. 7,064,244; WO 96/034096; and WO96/033735.

In another embodiment, a binding protein is a monoclonal antibody obtained from a nonhuman animal, and then modified (e.g., chimeric, humanized, deimmunized) using recombinant DNA techniques known in the art. A variety of approaches for making chimeric antibodies have been described (see, e.g., Morrison et al. (1985) Proc. Natl. Acad. Sci. USA 81(21):6851-55; Takeda et al. (1985) Nature 314(6010):452-54; U.S. Pat. No. 4,816,567; U.S. Pat. No. 4,816,397; European Patent Publication EP 0 171 496; European Patent Publication EP 0 173 494; and United Kingdom Patent GB 2 177 096).

Humanized binding proteins may be produced, for example, using transgenic mice that express human heavy and light chain genes, but are incapable of expressing the endogenous mouse immunoglobulin heavy and light chain genes. Winter (U.S. Pat. No. 5,225,539) describes an exemplary CDR-grafting method that may be used to prepare humanized binding proteins as described herein. All of the CDRs of a particular human binding protein may be replaced with at least a portion of a nonhuman CDR, or only some of the CDRs may be replaced with nonhuman CDRs. It is only necessary to replace the number of CDRs required for binding of the humanized binding protein to a predetermined antigen.

Humanized binding proteins or fragments thereof can be generated by replacing sequences of the Fv variable domain that are not directly involved in antigen binding with equivalent sequences from human Fv variable domains. Exemplary methods for generating humanized binding proteins or fragments thereof are provided by, e.g., Morrison (1985) Science 229:1202-07; Oi et al. (1986) BioTechniques 4:214; and U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing a binding protein, e.g., an antibody, against a predetermined target, as described above, as well as from other sources. The recombinant DNA encoding the humanized binding protein molecule can then be cloned into an appropriate expression vector.

In certain embodiments, a humanized binding protein is optimized by the introduction of conservative substitutions, consensus sequence substitutions, germline substitutions and/or backmutations. Such altered immunoglobulin molecules can be made by any of several techniques known in the art, (see, e.g., Teng et al. (1983) Proc. Natl. Acad. Sci. USA 80:7308-73; Kozbor et al. (1983) Immunol. Today 4:7279; Olsson et al. (1982) Meth. Enzymol. 92:3-16); PCT Publication WO 92/006193; and EP 0 239 400).

A binding protein or fragment thereof may also be modified by specific deletion of human T cell epitopes or “deimmunization” by the methods disclosed in, e.g., WO 98/052976 and WO 00/034317. Briefly, the heavy and light chain variable domains of an antibody can be analyzed for peptides that bind to MHC Class II; these peptides represent potential T cell epitopes (as defined in, e.g., WO 98/052976 and WO 00/034317). For detection of potential T cell epitopes, a computer modeling approach termed “peptide threading” can be applied and, in addition, a database of human MHC Class II binding peptides can be searched for motifs present in the V_{H} and V_{L} sequences, as described in, e.g., WO 98/052976 and WO 00/034317. These motifs bind to any of the 18 major MHC Class II DR allotypes, and thus constitute potential T cell epitopes. Potential T cell epitopes detected can be eliminated by substituting small numbers of amino acid residues in the variable domains or by single amino acid substitutions. Typically, conservative substitutions are made. Often, but not exclusively, an amino acid common to a position in human germline antibody sequences may be used. Human germline sequences are disclosed in, e.g., Tomlinson et al. (1992) J. Mol. Biol. 227:776-98; Cook et al. (1995) Immunol. Today 16(5):237-42; Chothia et al. (1992) J. Mol. Biol. 227:799-817; and Tomlinson et al. (1995) EMBO J. 14:4628-38. The V BASE directory provides a comprehensive directory of human immunoglobulin variable region sequences (compiled by Tomlinson et al., MRC Centre for Protein Engineering, Cambridge, UK). These sequences can be used as a source of human sequence, e.g., for framework regions and CDRs. Consensus human framework regions can also be used, as described in, e.g., U.S. Pat. No. 6,300,064.

The term “human binding protein” includes binding proteins having variable and constant regions corresponding substantially to human germline immunoglobulin sequences known in the art, including, for example, those described by Kabat et al. (5th ed. 1991) Sequences of Proteins of Immunological Interest, U.S. Department of Health and Human Services, NIH Publication No. 91-3242. The human binding proteins of the invention (e.g., human antibodies) may include amino acid residues not encoded by human germline immunoglobulin sequences (e.g., mutations introduced by random or site-specific mutagenesis in vitro or by somatic mutation in vivo), for example, in the CDRs, and in particular, CDR3. The human binding proteins can have at least one, two, three, four, five, or more positions replaced with an amino acid residue that is not encoded by the human germline immunoglobulin sequence.

Regions of the binding proteins, e.g., constant regions of the antibodies, can be altered, e.g., mutated, to modify the properties of the antibody (e.g., to increase or decrease one or more of: Fc receptor binding, antibody glycosylation, the number of cysteine residues, effector cell function, or complement function).

In certain embodiments, a binding protein can contain an altered immunoglobulin constant or Fc region. For example, binding proteins may bind more strongly or with more specificity to effector molecules such as complement and/or Fc receptors, which can control several immune functions of the binding protein such as effector cell activity, lysis, complement-mediated activity, binding protein clearance, and binding protein half-life. Typical Fc receptors that bind to an Fc region of a binding protein (e.g., an IgG antibody) include, but are not limited to, receptors of the FcγRI, FcγRII, and FcRn subclasses, including allelic variants and alternatively spliced forms of these receptors. Fc receptors are reviewed in, e.g., Ravetch and Kinet (1991) Annu. Rev. Immunol. 9:457-92; Capel et al. (1994) Immunomethods 4:25-34; and de Haas et al. (1995) J. Lab. Clin. Med. 126:330-41.

The term “single domain binding protein” as used herein includes any single domain-binding scaffold that binds to an antigen, protein, or polypeptide. Single domain binding proteins can include any natural, recombinant, synthetic, or genetically engineered protein scaffold, or a combination thereof, that binds an antigen or fragment thereof to form a complex and elicit a biological response (e.g., agonize or antagonize a particular biological activity). Single domain binding proteins may be derived from naturally occurring proteins or antibodies, or they can be synthetically engineered or produced by recombinant technology. In certain embodiments of the invention, single domain binding proteins include binding proteins wherein the CDRs are part of a single domain polypeptide. Examples include, but are not limited to, heavy chain binding proteins, binding proteins naturally devoid of light chains, single domain binding proteins derived from conventional four-chain antibodies, engineered binding proteins, and single domain scaffolds other than those derived from antibodies. Single domain binding proteins include any known in the art, as well as any future-determined or -learned single domain binding proteins. Single domain binding proteins may be derived from any species including, but not limited to mouse, rat, human, camel, llama, fish, shark, goat, rabbit, chicken, and bovine. In one aspect of the invention, the single domain binding protein can be derived from a variable region of the immunoglobulin found in fish, such as, for example, that which is derived from the immunoglobulin isotype known as Novel Antigen Receptor (NAR) found in the serum of shark. Methods of producing single domain binding proteins derived from a variable region of NAR (IgNARs) are described in, e.g., WO 03/014161 and Streltsov (2005) Protein Sci. 14:2901-09.

Single domain binding proteins also include naturally occurring single domain binding proteins known in the art as heavy chain antibodies devoid of light chains. This variable domain derived from a heavy chain antibody naturally devoid of a light chain is known herein as a VHH, or a nanobody, to distinguish it from the conventional V_{H} of four-chain immunoglobulins. Such a VHH molecule can be derived from antibodies raised in Camelidae species, for example, in camel, llama, dromedary, alpaca and guanaco, and is sometimes called a camelid or camelized variable domain (see, e.g., Muyldermans (2001) J. Biotechnology 74(4):277-302, incorporated herein by reference). Other species besides those in the family Camelidae may also produce heavy chain binding proteins naturally devoid of light chains. VHH molecules are about ten times smaller than IgG molecules. They are single polypeptides and are very stable, resisting extreme pH and temperature conditions. Moreover, they are resistant to the action of proteases, which is not the case for conventional antibodies. Furthermore, in vitro expression of VHHs produces high yield, properly folded functional VHHs. In addition, binding proteins generated in camelids will recognize epitopes other than those recognized by antibodies generated in vitro via antibody libraries or via immunization of mammals other than camelids (see, e.g., WO 97/049805 and WO 94/004678, which are incorporated herein by reference).

A “bispecific” or “bifunctional” binding protein is an artificial hybrid binding protein having two different heavy/light chain pairs and two different binding sites. Bispecific binding proteins can be produced by a variety of methods including fusion of hybridomas or linking of Fab′ fragments (see, e.g., Songsivilai and Lachmann (1990) Clin. Exp. Immunol. 79:315-21; Kostelny et al. (1992) J. Immunol. 148:1547-53. In one embodiment, the bispecific binding protein comprises a first binding domain polypeptide, such as an Fab′ fragment, linked via an immunoglobulin constant region to a second binding domain polypeptide.

Binding proteins of the invention can also comprise peptide mimetics. Peptide mimetics are peptide-containing molecules that mimic elements of protein secondary structure (see, for example, Johnson et al., Peptide Turn Mimetics in: Biotechnology and Pharmacy (1993) Pezzuto et al., Eds., Chapman and Hall, New York, incorporated by reference herein in its entirety). The underlying rationale behind the use of peptide mimetics is that the peptide backbone of proteins exists chiefly to orient amino acid side chains in such a way as to facilitate molecular interactions, such as those between antibody and antigen. A peptide mimetic is expected to permit molecular interactions similar to the natural molecule. These principles may be used to engineer second-generation molecules having many of the natural properties of the targeting peptides disclosed herein, but with altered and potentially improved characteristics.

Other embodiments of binding proteins include fusion proteins. These molecules generally have all or a substantial portion of a targeting peptide, for example, IL21R or an anti IL21R binding protein, linked at the N- or C-terminus, to all or a portion of a second polypeptide or protein. For example, fusion proteins may employ leader sequences from other species to permit the recombinant expression of a protein in a heterologous host. Another useful fusion includes the addition of an immunologically active domain, such as a binding protein epitope, to facilitate purification of the fusion protein. Inclusion of a cleavage site at or near the fusion junction will facilitate removal of the extraneous polypeptide after purification. Other useful fusions include the linking of functional domains, such as active sites from enzymes, glycosylation domains, cellular targeting signals, or transmembrane regions. Examples of proteins or peptides that may be incorporated into a fusion protein include, but are not limited to, cytostatic proteins, cytocidal proteins, pro-apoptotic agents, anti-angiogenic agents, hormones, cytokines, growth factors, peptide drugs, antibodies, Fab fragments of antibodies, antigens, receptor proteins, enzymes, lectins, MHC proteins, cell adhesion proteins, and binding proteins. Methods of generating fusion proteins are well known to those of skill in the art. Such proteins can be produced, for example, by chemical attachment using bifunctional cross-linking reagents, by de novo synthesis of the complete fusion protein, or by attachment of a DNA sequence encoding the targeting peptide to a DNA sequence encoding the second peptide or protein, followed by expression of the intact fusion protein.

Binding proteins can also include binding domain-immunoglobulin fusion proteins, including a binding domain polypeptide that is fused or otherwise connected to an immunoglobulin hinge or hinge-acting region polypeptide, which in turn is fused or otherwise connected to a region comprising one or more native or engineered constant regions from an immunoglobulin heavy chain other than C_{H}1, for example, the C_{H}2 and C_{H}3 regions of IgG and IgA, or the C_{H}3 and C_{H}4 regions of IgE (see, e.g., Ledbetter et al., U.S. Patent Application Publication 2005/0136049, for a more complete description). The binding domain-immunoglobulin fusion protein can further include a region that includes a native or engineered immunoglobulin heavy chain C_{H}2 constant region polypeptide (or C_{H}3 in the case of a construct derived in whole or in part from IgE) that is fused or otherwise connected to the hinge region polypeptide, and a native or engineered immunoglobulin heavy chain C_{H}3 constant region polypeptide (or C_{H}4 in the case of a construct derived in whole or in part from IgE) that is fused or otherwise connected to the C_{H}2 constant region polypeptide (or C_{H}3 in the case of a construct derived in whole or in part from IgE). Typically, such binding domain-immunoglobulin fusion proteins are capable of at least one immunological activity selected from the group consisting of antibody-dependent cell-mediated cytotoxicity, complement fixation, and/or binding to a target, for example, a target antigen. The binding proteins of the invention can be derived from any species including, but not limited to mouse, rat, human, camel, llama, fish, shark, goat, rabbit, chicken, and bovine.

In one embodiment of a fusion protein, the targeting peptide, for example, IL21R, is fused with an immunoglobulin heavy chain constant region, such as an Fc fragment, which contains two constant region domains and a hinge region, but lacks the variable region (see, e.g., U.S. Pat. Nos. 6,018,026 and 5,750,375, incorporated by reference herein). The Fc region may be a naturally occurring Fc region, or may be altered to improve certain qualities, e.g., therapeutic qualities, circulation time, reduced aggregation. Peptides and proteins fused to an Fc region typically exhibit a greater half-life in vivo than the unfused counterpart does. In addition, a fusion to an Fc region permits dimerization/multimerization of the fusion polypeptide.

For additional binding protein/antibody production techniques, see, e.g., Antibodies: A Laboratory Manual, eds. Harlow et al., Cold Spring Harbor Laboratory (1988). The present invention is not necessarily limited to any particular source, method of production, or other special characteristics of a binding protein or an antibody.

In addition, one of skill in the art will appreciate that modifications to a binding protein as described herein are not exhaustive, and that many other modifications will be obvious to a skilled artisan in light of the teachings of the present disclosure. Many modifications are described in detail in, e.g., U.S. patent application Ser. No. 12/472,237.

The term “neutralizing” refers to a binding protein or antigen-binding fragment thereof (for example, an antibody) that reduces or blocks the activity of a signaling pathway or an antigen, e.g., IL21/IL21R signaling pathway or IL21R antigen. “An anti-product antibody,” as used herein, refers to an antibody formed in response to exogenous protein, e.g., an anti-IL21R antibody. “A neutralizing anti-product antibody,” as used herein, refers to an anti-product antibody that blocks the in vivo activity of the exogenously introduced protein, e.g., an anti-IL21R antibody. In some embodiments of the invention, a neutralizing anti-product antibody diminishes in vivo activity of an IL21R antibody, e.g., in vivo pharmacodynamic (PD) activity of an IL21R antibody (such as the ability of an anti-IL21R antibody to modulate expression of IL21-responsive cytokines or genes).

The term “effective amount” refers to a dosage or amount that is sufficient to regulate IL21R activity to ameliorate or lessen the severity of clinical symptoms or achieve a desired biological outcome, e.g., decreased T cell and/or B cell activity, suppression of autoimmunity, suppression of transplant rejection.

The phrases “inhibit,” “antagonize,” “block,” or “neutralize” IL21R activity and its cognates refer to a reduction, inhibition, or otherwise diminution of at least one activity of IL21R due to binding an anti-IL21R binding protein, wherein the reduction is relative to the activity of IL21R in the absence of the same binding protein. The IL21R activity can be measured using any technique known in the art. Inhibition or antagonism does not necessarily indicate a total elimination of the IL21R biological activity. A reduction in activity may be about 10%, 20%, 30%, 40%, 50%, 60%, 70%, 80%, 90%, 95%, or more.

In one embodiment of the invention, at least one activity mediated through IL21R is the effect in PBMCs of IL21 on gene expression, with significant elevations in RNA levels observed under at least one condition tested for CCL19, CCL2, CCL3, CCR2, CD19, CD40, CSF2, CSF3, CXCL10, CXCL11, GZMB, IFNγ, IL10, IL12β, IL1β, IL2RA, IL6, PRF1, PTGS2, and TBX21. The most robust IL21-dependent RNA responses observed in PBMCs under the culture tested were of GZMB, IFNγ, IL2RA, PRF1, and IL6, and at the longer time periods tested IL10.

The term “modulate,” as used herein, refers to any substantial increase such as a change in expression of at least one IL21-responsive gene. A skilled artisan will understand that if, in the absence of anti-IL21R binding protein, IL21 upregulates the level of expression of an IL21-responsive gene, inhibition of IL21R activity (e.g., with an anti-IL21R binding protein) will lead to blocking or inhibition of expression of the IL21-responsive gene. Alternatively, if in the absence of anti-IL21R binding protein, IL21 decreases the level of expression of an IL21-responsive gene, inhibition of IL21R activity will lead to restoration or increase of expression of the IL21-responsive gene.

As used herein, “in vitro-generated binding protein,” e.g., “in vitro-generated antibody” refers to a binding protein/antibody where all or part of the variable region (e.g., at least one CDR) is generated in a nonimmune cell selection (e.g., an in vitro phage display, protein chip, or any other method in which candidate sequences can be tested for their ability to bind to an antigen).

The term “isolated” refers to a molecule that is substantially free of its natural environment. For instance, an isolated protein is substantially free of cellular material or other proteins from the cell or tissue source from which it was derived. The term also refers to preparations where the isolated protein is sufficiently pure for pharmaceutical compositions, or is at least 70-80% (w/w) pure, at least 80-90% (w/w) pure, at least 90-95% (w/w) pure, or at least 95%, 96%, 97%, 98%, 99%, or 100% (w/w) pure.

The phrase “percent identical” or “percent identity” refers to the similarity between at least two different sequences. This percent identity can be determined by standard alignment algorithms, for example, the Basic Local Alignment Search Tool (BLAST) described by Altshul et al. ((1990) J. Mol. Biol. 215:403-10); the algorithm of Needleman et al. ((1970) J. Mol. Biol. 48:444-53); or the algorithm of Meyers et al. ((1988) Comput. Appl. Biosci. 4:11-17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of Meyers and Miller ((1989) CABIOS 4:11-17), which has been incorporated into the ALIGN program (version 2.0), using a PAM120 weight residue table, a gap length penalty of 12, and a gap penalty of 4. The percent identity is usually calculated by comparing sequences of similar length.

The term “repertoire” refers to at least one nucleotide sequence derived wholly or partially from at least one sequence encoding at least one immunoglobulin.

The sequence(s) may be generated by rearrangement in vivo of the V, D, and J segments of heavy chains, and the V and J segments of light chains. Alternatively, the sequence(s) can be generated from a cell in response to which rearrangement occurs, e.g., in vitro stimulation. Alternatively, part or all of the sequence(s) may be obtained by DNA splicing, nucleotide synthesis, mutagenesis, or other methods (see, e.g., U.S. Pat. No. 5,565,332). A repertoire may include only one sequence or may include a plurality of sequences, including ones in a genetically diverse collection.

The terms “specific binding,” “specifically binds,” and the like refer to two molecules forming a complex that is relatively stable under physiologic conditions. Specific binding is characterized by a high affinity and a low-to-moderate capacity as distinguished from nonspecific binding, which usually has a low affinity with a moderate-to-high capacity. Typically, binding is considered specific when the association constant Ka is higher than about 10⁶ M⁻¹s⁻¹. If necessary, nonspecific binding can be reduced without substantially affecting specific binding by varying the binding conditions. The appropriate binding conditions, such as concentration of binding protein, ionic strength of the solution, temperature, time allowed for binding, concentration of a blocking agent (e.g., serum albumin or milk casein), etc., can be improved by a skilled artisan using routine techniques. Illustrative conditions are set forth herein, but other conditions known to the person of ordinary skill in the art fall within the scope of this invention.

As used herein, the terms “stringent,” “stringency,” and the like describe conditions for hybridization and washing. The isolated polynucleotides of the present invention can be used as hybridization probes and primers to identify and isolate nucleic acids having sequences identical to or similar to those encoding the disclosed polynucleotides. Therefore, polynucleotides isolated in this fashion may be used to produce binding proteins against IL21R or to identify cells expressing such binding proteins. Hybridization methods for identifying and isolating nucleic acids include polymerase chain reaction (PCR), Southern hybridizations, in situ hybridization and Northern hybridization, and are well known to those skilled in the art.

Hybridization reactions can be performed under conditions of different stringencies. The stringency of a hybridization reaction includes the difficulty with which any two nucleic acid molecules will hybridize to one another and the conditions under which they will remain hybridized. Preferably, each hybridizing polynucleotide hybridizes to its corresponding polynucleotide under reduced stringency conditions, more preferably stringent conditions, and most preferably highly stringent conditions. Stringent conditions are known to those skilled in the art and can be found in, e.g., Current Protocols in Molecular Biology, John Wiley & Sons, N.Y. (1989) 6.3.1-6.3.6. Both aqueous and nonaqueous methods are described in this reference, and either can be used. One example of stringent hybridization conditions is hybridization in 6× sodium chloride/sodium citrate (SSC) at about 45° C., followed by at least one wash in 0.2×SSC/0.1% SDS at 50° C. Stringent hybridization conditions are also accomplished with wash(es) in, e.g., 0.2×SSC/0.1% SDS at 55° C., 60° C., or 65° C. Highly stringent conditions include, e.g., hybridization in 0.5M sodium phosphate/7% SDS at 65° C., followed by at least one wash at 0.2×SSC/1% SDS at 65° C. Further examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 1
Hybridization Conditions

| | | Hybrid | Hybridization | Wash |
|---|---|---|---|---|
| | | Length | Temperature and | Temperature and |
| Condition | Hybrid | (bp)¹ | Buffer² | Buffer² |
| A | DNA:DNA | >50 | 65° C.; 1X SSC | 65° C.; 0.3X SSC |
| | | | -or- | |
| | | | 42° C.; 1X SSC, | |
| | | | 50% formamide | |
| B | DNA:DNA | <50 | T_{B}*; 1X SSC | T_{B}*; 1X SSC |
| C | DNA:RNA | >50 | 67° C.; 1X SSC | 67° C.; 0.3X SSC |
| | | | -or- | |
| | | | 45° C.; 1X SSC, | |
| | | | 50% formamide | |
| D | DNA:RNA | <50 | T_{D}*; 1X SSC | T_{D}*; 1X SSC |
| E | RNA:RNA | >50 | 70° C.; 1X SSC | 70° C.; 0.3X SSC |
| | | | -or- | |
| | | | 50° C.; 1X SSC, | |
| | | | 50% formamide | |
| F | RNA:RNA | <50 | T_{F}*; 1X SSC | T_{F}*; 1X SSC |
| G | DNA:DNA | >50 | 65° C.; 4X SSC | 65° C.; 1X SSC |
| | | | -or- | |
| | | | 42° C.; 4X SSC, | |
| | | | 50% formamide | |
| H | DNA:DNA | <50 | T_{H}*; 4X SSC | T_{H}*; 4X SSC |
| I | DNA:RNA | >50 | 67° C.; 4X SSC | 67° C.; 1X SSC |
| | | | -or- | |
| | | | 45° C.; 4X SSC, | |
| | | | 50% formamide | |
| J | DNA:RNA | <50 | T_{J}*; 4X SSC | T_{J}*; 4X SSC |
| K | RNA:RNA | >50 | 70° C.; 4X SSC | 67° C.; 1X SSC |
| | | | -or- | |
| | | | 50° C.; 4X SSC, | |
| | | | 50% formamide | |
| L | RNA:RNA | <50 | T_{L}*; 2X SSC | T_{L}*; 2X SSC |
| M | DNA:DNA | >50 | 50° C.; 4X SSC | 50° C.; 2X SSC |
| | | | -or- | |
| | | | 40° C.; 6X SSC, | |
| | | | 50% formamide | |
| N | DNA:DNA | <50 | T_{N}*; 6X SSC | T_{N}*; 6X SSC |
| O | DNA:RNA | >50 | 55° C.; 4X SSC | 55° C.; 2X SSC |
| | | | -or- | |
| | | | 42° C.; 6X SSC, | |
| | | | 50% formamide | |
| P | DNA:RNA | <50 | T_{P}*; 6X SSC | T_{P}*; 6X SSC |
| Q | RNA:RNA | >50 | 60° C.; 4X SSC | 60° C.; 2X SSC |
| | | | -or- | |
| | | | 45° C.; 6X SSC, | |
| | | | 50% formamide | |
| R | RNA:RNA | <50 | T_{R}*; 4X SSC | T_{R}*; 4X SSC |

¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
²SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 min after hybridization is complete.
T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length,Tₘ(° C.) = 81.5 + 16.6(log₁₀Na⁺) + 0.41(% G + C) − (600/N), where N is the number of bases in the hybrid, and Na⁺ is the concentration of sodium ions in the hybridization buffer (Na⁺ for 1X SSC = 0.165 M).
Additional examples of stringency conditions for polynucleotide hybridization are provided in Sambrook et al., Molecular Cloning: A Laboratory Manual, Chs. 9 & 11, Cold Spring Harbor Laboratory Press, Cold Spring Harbor, NY (1989), and Ausubel et al., eds., Current Protocols in Molecular Biology, Sects. 2.10 & 6.3-6.4, John Wiley & Sons, Inc. (1995), herein incorporated by reference.

The isolated polynucleotides of the present invention may be used as hybridization probes and primers to identify and isolate DNAs having sequences encoding allelic variants of the disclosed polynucleotides. Allelic variants are naturally occurring alternative forms of the disclosed polynucleotides that encode polypeptides that are identical to or have significant similarity to the polypeptides encoded by the disclosed polynucleotides. Preferably, allelic variants have at least about 90% sequence identity (more preferably, at least about 95% identity; most preferably, at least about 99% identity) with the disclosed polynucleotides. The isolated polynucleotides of the present invention may also be used as hybridization probes and primers to identify and isolate DNAs having sequences encoding polypeptides homologous to the disclosed polynucleotides. These homologs are polynucleotides and polypeptides isolated from a different species than that of the disclosed polypeptides and polynucleotides, or within the same species, but with significant sequence similarity to the disclosed polynucleotides and polypeptides. Preferably, polynucleotide homologs have at least about 50% sequence identity (more preferably, at least about 75% identity; most preferably, at least about 90% identity) with the disclosed polynucleotides, whereas polypeptide homologs have at least about 30% sequence identity (more preferably, at least about 45% identity; most preferably, at least about 60% identity) with the disclosed binding proteins/polypeptides. Preferably, homologs of the disclosed polynucleotides and polypeptides are those isolated from mammalian species. The isolated polynucleotides of the present invention may additionally be used as hybridization probes and primers to identify cells and tissues that express the binding proteins of the present invention and the conditions under which they are expressed.

The phrases “substantially as set out,” “substantially identical,” and “substantially homologous” mean that the relevant amino acid or nucleotide sequence (e.g., CDR(s), V_{H}, or V_{L} domain(s)) will be identical to or have insubstantial differences (e.g., through conserved amino acid substitutions) in comparison to the sequences which are set out. Insubstantial differences include minor amino acid changes, such as one or two substitutions in a five amino acid sequence of a specified region. For example, in the case of antibodies, the second antibody has the same specificity and has at least about 50% of the affinity of the first antibody.

Sequences substantially identical or homologous to the sequences disclosed herein are also part of this application. In some embodiments, the sequence identity can be about 85%, 90%, 95%, 96%, 97%, 98%, 99%, or higher. Alternatively, substantial identity or homology exists when the nucleic acid segments will hybridize under selective hybridization conditions (e.g., highly stringent hybridization conditions), to the complement of the strand. The nucleic acids may be present in whole cells, in a cell lysate, or in a partially purified or substantially pure form.

The term “therapeutic agent” or the like is a substance that treats or assists in treating a medical disorder or symptoms thereof. Therapeutic agents may include, but are not limited to, substances that modulate immune cells or immune responses in a manner that complements the use of anti-IL21R binding proteins. In one embodiment of the invention, a therapeutic agent is a therapeutic binding protein, e.g., a therapeutic antibody, e.g., an anti-IL21R antibody. In another embodiment of the invention, the therapeutic agent is a therapeutic binding protein, e.g., an anti-IL21R nanobody. Nonlimiting examples and uses of therapeutic agents are described herein.

As used herein, a “therapeutically effective amount” of an anti-IL21R binding protein refers to an amount of the binding protein that is effective, upon single or multiple dose administration to a subject (such as a human patient), for treating, preventing, curing, delaying, reducing the severity of, and/or ameliorating at least one symptom of a disorder or a recurring disorder, or prolonging the survival of the subject beyond that expected in the absence of such treatment. In one embodiment, a therapeutically effective amount may be an amount of an anti-IL21R binding protein that is sufficient to modulate expression of at least one IL21-responsive cytokine or gene.

The term “safety study species” refers to a species in which the binding protein has the desired biological activity, allowing a valid comparison with another mammalian species for safety. For example, a suitable safety study species may be a primate, e.g., a cynomolgus monkey.

The term “treatment” refers to a therapeutic or preventative measure. The treatment may be administered to a subject who has a medical disorder or who ultimately may acquire the disorder, in order to prevent, cure, delay, reduce the severity of, and/or ameliorate one or more symptoms of a disorder or a recurring disorder, or in order to prolong the survival of a subject beyond that expected in the absence of such treatment.

The term “cytokine storm” refers to a series of events that result in a devastating and potentially fatal immune reaction that comprises a positive feedback loop between cytokines and immune cells that in turn leads to highly elevated levels of various cytokines. Cytokines that are induced during cytokine storm include, e.g., one or more of the following: IL4, IL2, IL1β, IL12, TNF, IFNγ, IL6, IL8, and IL10.

### Anti-IL21R Binding Proteins

The disclosure of the present application, and further in conjunction with the disclosure of U.S. application Ser. No. 12/472,237 (incorporated by reference herein in its entirety), provides novel anti-IL21R binding proteins that comprise novel antigen-binding fragments. The disclosure also provides novel CDRs that have been derived from human immunoglobulin gene libraries. The protein structure that is generally used to carry a CDR is an antibody heavy or light chain or a portion thereof, wherein the CDR is localized to a region associated with a naturally occurring CDR. The structures and locations of variable domains may be determined as described in Kabat et al. ((1991) supra).

Illustrative embodiments of binding proteins (and antigen-binding fragments thereof) related to the present invention are identified as AbA-AbU, H3-H6, L1-L6, L8-L21, and L23-L25. DNA and amino acid sequences of these nonlimiting illustrative embodiments of anti-IL21R binding proteins are set forth in SEQ ID NOs:5-195, 213-229, and 239-248. DNA and amino acid sequences of some illustrative embodiments of anti-IL21R binding proteins, including their scFv fragments, V_{H} and V_{L} domains, and CDRs, as well as their present codes and previous designations, are set forth in Tables 2A and 2B, and are addressed in detail in U.S. patent application Ser. No. 12/472,237 (incorporated by reference herein).

TABLE 2A
Correlation of Present Antibody Codes and
Previous Designations

| Present Code | Previous Designation |
|---|---|
| AbA | VHP/VL2 |
| AbB | VHP/VL3 |
| AbC | VHP/VL11 |
| AbD | VHP/VL13 |
| AbE | VHP/VL14 |
| AbF | VHP/VL17 |
| AbG | VHP/VL18 |
| AbH | VHP/VL19 |
| AbI | VHP/VL24 |
| AbJ | VH3/VLP |
| AbK | VH3/VL3 |
| AbL | VH3/VL13 |
| AbM | VH6/VL13 |
| AbN | VH6/VL24 |
| AbO | VHP/VL16; VHPTM/VL16 |
| AbP | VHP/VL20; VHPTM/VL20 |
| AbQ | VH3/VL2; VH3DM/VL2 |
| AbR | VH3/VL18; VH3DM/VL18 |
| AbS | VHP/VL6; VHPTM/VL6; VL6 |
| AbT | VHP/VL9; VHPTM/VL9; VL9 |
| AbU | VHP/VL25; VHPTM/VL25 |
| AbV | VH3TM/VL2 |
| AbW | VH3TM/VL18 |
| AbX | VHPDM/VL9 |
| AbY | VHPg4/VL9 |
| AbZ | VHPWT/VL9 |

TABLE 2B
Amino Acid and Nucleotide Sequences of V_{H} and V_{L} Domains, scFv,
and CDRs of Illustrative Binding Proteins of the Invention

| | | | | | H6 | L1 |
|---|---|---|---|---|---|---|
| REGION | TYPE | H3 SEQ ID | H4 SEQ ID | H5 SEQ ID | SEQ ID | SEQ ID |
| V_{H} | AA | NO: 14 | NO: 16 | NO: 18 | NO: 20 | NO: 6 |
| V_{L} | AA | NO: 10 | NO: 10 | NO: 10 | NO: 10 | NO: 22 |
| scFv | AA | NO: 110 | NO: 112 | NO: 114 | NO: 116 | NO: 118 |
| CDR H1 | AA | NO: 163 | NO: 163 | NO: 163 | NO: 163 | NO: 163 |
| CDR H2 | AA | NO: 164 | NO: 164 | NO: 164 | NO: 164 | NO: 164 |
| CDR H3 | AA | NO: 165 | NO: 166 | NO: 167 | NO: 168 | NO: 169 |
| CDR L1 | AA | NO: 194 | NO: 194 | NO: 194 | NO: 194 | NO: 194 |
| CDR L2 | AA | NO: 195 | NO: 195 | NO: 195 | NO: 195 | NO: 195 |
| CDR L3 | AA | NO: 170 | NO: 170 | NO: 170 | NO: 170 | NO: 171 |
| V_{H} | DNA | NO: 13 | NO: 15 | NO: 17 | NO: 19 | NO: 5 |
| V_{L} | DNA | NO: 9 | NO: 9 | NO: 9 | NO: 9 | NO: 21 |
| scFv | DNA | NO: 109 | NO: 111 | NO: 113 | NO: 115 | NO: 117 |
| | | L2 | L3 | L4 | L5 | L6 |
| REGION | TYPE | SEQ ID | SEQ ID | SEQ ID | SEQ ID | SEQ ID |
| V_{H} | AA | NO: 6 | NO: 6 | NO: 6 | NO: 6 | NO: 6 |
| V_{L} | AA | NO: 24 | NO: 26 | NO: 28 | NO: 30 | NO: 32 |
| scFv | AA | NO: 120 | NO: 122 | NO: 124 | NO: 126 | NO: 128 |
| CDR H1 | AA | NO: 163 | NO: 163 | NO: 163 | NO: 163 | NO: 163 |
| CDR H2 | AA | NO: 164 | NO: 164 | NO: 164 | NO: 164 | NO: 164 |
| CDR H3 | AA | NO: 169 | NO: 169 | NO: 169 | NO: 169 | NO: 169 |
| CDR L1 | AA | NO: 194 | NO: 194 | NO: 194 | NO: 194 | NO: 194 |
| CDR L2 | AA | NO: 195 | NO: 195 | NO: 195 | NO: 195 | NO: 195 |
| CDR L3 | AA | NO: 172 | NO: 173 | NO: 174 | NO: 175 | NO: 176 |
| V_{H} | DNA | NO: 5 | NO: 5 | NO: 5 | NO: 5 | NO: 5 |
| V_{L} | DNA | NO: 23 | NO: 25 | NO: 27 | NO: 29 | NO: 31 |
| scFv | DNA | NO: 119 | NO: 121 | NO: 123 | NO: 125 | NO: 127 |
| | | L8 | L9 | L10 | L11 | L12 |
| REGION | TYPE | SEQ ID | SEQ ID | SEQ ID | SEQ ID | SEQ ID |
| V_{H} | AA | NO: 6 | NO: 6 | NO6 | NO: 6 | NO: 6 |
| V_{L} | AA | NO: 34 | NO: 36 | NO: 38 | NO: 40 | NO: 42 |
| scFv | AA | NO: 130 | NO: 132 | NO: 134 | NO: 136 | NO: 138 |
| CDR H1 | AA | NO: 163 | NO: 163 | NO: 163 | NO: 163 | NO: 163 |
| CDR H2 | AA | NO: 164 | NO: 164 | NO: 164 | NO: 164 | NO: 164 |
| CDR H3 | AA | NO: 169 | NO: 169 | NO: 169 | NO: 169 | NO: 169 |
| CDR L1 | AA | NO: 194 | NO: 194 | NO: 194 | NO: 194 | NO: 194 |
| CDR L2 | AA | NO: 195 | NO: 195 | NO: 195 | NO: 195 | NO: 195 |
| CDR L3 | AA | NO: 177 | NO: 178 | NO: 179 | NO: 180 | NO: 181 |
| V_{H} | DNA | NO: 5 | NO: 5 | NO: 5 | NO: 5 | NO: 5 |
| V_{L} | DNA | NO: 33 | NO: 35 | NO: 37 | NO: 39 | NO: 41 |
| scFv | DNA | NO: 129 | NO: 131 | NO: 133 | NO: 135 | NO: 137 |
| | | L13 | L14 | L15 | L16 | L17 |
| REGION | TYPE | SEQ ID | SEQ ID | SEQ ID | SEQ ID | SEQ ID |
| V_{H} | AA | NO: 6 | NO: 6 | NO: 6 | NO: 6 | NO: 6 |
| V_{L} | AA | NO: 44 | NO: 46 | NO: 48 | NO: 50 | NO: 52 |
| scFv | AA | NO: 140 | NO: 142 | NO: 144 | NO: 146 | NO: 148 |
| CDR H1 | AA | NO: 163 | NO: 163 | NO: 163 | NO: 163 | NO: 163 |
| CDR H2 | AA | NO: 164 | NO: 164 | NO: 164 | NO: 164 | NO: 164 |
| CDR H3 | AA | NO: 169 | NO: 169 | NO: 169 | NO: 169 | NO: 169 |
| CDR L1 | AA | NO: 194 | NO: 194 | NO: 194 | NO: 194 | NO: 194 |
| CDR L2 | AA | NO: 195 | NO: 195 | NO: 195 | NO: 195 | NO: 195 |
| CDR L3 | AA | NO: 182 | NO: 183 | NO: 184 | NO: 185 | NO: 186 |
| V_{H} | DNA | NO: 5 | NO: 5 | NO: 5 | NO: 5 | NO: 5 |
| V_{L} | DNA | NO: 43 | NO: 45 | NO: 47 | NO: 49 | NO: 51 |
| scFv | DNA | NO: 139 | NO: 141 | NO: 143 | NO: 145 | NO: 147 |
| | | L18 | L19 | L20 | L21 | L23 |
| REGION | TYPE | SEQ ID | SEQ ID | SEQ ID | SEQ ID | SEQ ID |
| V_{H} | AA | NO: 6 | NO: 6 | NO: 6 | NO: 6 | NO: 6 |
| V_{L} | AA | NO: 54 | NO: 56 | NO: 58 | NO: 60 | NO: 62 |
| scFv | AA | NO: 150 | NO: 152 | NO: 154 | NO: 156 | NO: 158 |
| CDR H1 | AA | NO: 163 | NO: 163 | NO: 163 | NO: 163 | NO: 163 |
| CDR H2 | AA | NO: 164 | NO: 164 | NO: 164 | NO: 164 | NO: 164 |
| CDR H3 | AA | NO: 169 | NO: 169 | NO: 169 | NO: 169 | NO: 169 |
| CDR L1 | AA | NO: 194 | NO: 194 | NO: 194 | NO: 194 | NO: 194 |
| CDR L2 | AA | NO: 195 | NO: 195 | NO: 195 | NO: 195 | NO: 195 |
| CDR L3 | AA | NO: 187 | NO: 188 | NO: 189 | NO: 190 | NO: 191 |
| V_{H} | DNA | NO: 5 | NO: 5 | NO: 5 | NO: 5 | NO: 5 |
| V_{L} | DNA | NO: 53 | NO: 55 | NO: 57 | NO: 59 | NO: 61 |
| scFv | DNA | NO: 149 | NO: 151 | NO: 153 | NO: 155 | NO: 157 |

| | | L24 | L25 |
|---|---|---|---|
| REGION | TYPE | SEQ ID | SEQ ID |
| V_{H} | AA | NO: 6 | NO: 6 |
| V_{L} | AA | NO: 64 | NO: 66 |
| scFv | AA | NO: 160 | NO: 162 |
| CDR H1 | AA | NO: 163 | NO: 163 |
| CDR H2 | AA | NO: 164 | NO: 164 |
| CDR H3 | AA | NO: 169 | NO: 169 |
| CDR L1 | AA | NO: 194 | NO: 194 |
| CDR L2 | AA | NO: 195 | NO: 195 |
| CDR L3 | AA | NO: 192 | NO: 193 |
| V_{H} | DNA | NO: 5 | NO: 5 |
| V_{L} | DNA | NO: 63 | NO: 65 |
| scFv | DNA | NO: 159 | NO: 161 |

The present invention can be applied to any number of binding proteins, including isolated binding proteins or antigen-binding fragments thereof that bind to IL21R, in particular, human IL21R. In certain embodiments, the anti-IL21R binding protein, e.g., the anti-IL21R antibody, can have at least one of the several characteristics, including pharmacokinetic and pharmacodynamic characteristics, described in detail in U.S. patent application Ser. No. 12/472,237 (incorporated-by reference herein). For example, the anti-IL21R binding protein can modulate expression of IL21-responsive cytokines or IL21-responsive genes; and/or it may not activate cytokine storm genes when administered to subjects, e.g., human or cynomolgus monkey subjects.

### Therapeutic Uses of Anti-IL21R Binding Proteins

Anti-IL21R binding proteins that act as antagonists to IL21R can be used to regulate at least one IL21R-mediated immune response, such as one or more of cell proliferation, cytokine expression or secretion, chemokine secretion, and cytolytic activity, of T cells, B cells, NK cells, macrophages, or synovial cells. Accordingly, the disclosed binding proteins can be used to inhibit the activity (e.g., proliferation, differentiation, and/or survival) of an immune or hematopoietic cell (e.g., a cell of myeloid, lymphoid, or erythroid lineage, or precursor cells thereof), and, thus, can be used to treat, e.g., a variety of immune disorders, hyperproliferative disorders of the blood, and an acute phase response. Examples of immune disorders that can be treated include, but are not limited to, transplant rejection, graft-versus-host disease, allergies (for example, atopic allergy) and autoimmune diseases. Autoimmune diseases include diabetes mellitus, arthritic disorders (including rheumatoid arthritis, juvenile rheumatoid arthritis, osteoarthritis, psoriatic arthritis, and ankylosing spondylitis), spondyloarthropathy, multiple sclerosis, encephalomyelitis, myasthenia gravis, systemic lupus erythematosus, cutaneous lupus erythematosus, autoimmune thyroiditis, dermatitis (including atopic dermatitis and eczematous dermatitis), psoriasis, Sjögren's syndrome, IBD (including Crohn's disease and ulcerative colitis), asthma (including intrinsic asthma and allergic asthma), scleroderma and vasculitis.

### Diagnostic Uses of Anti-IL21R Binding Proteins

The binding proteins may also be used to detect the presence of IL21R in biological samples. By correlating the presence or level of these binding proteins with a medical condition, one of skill in the art can diagnose the associated medical condition. For example, stimulated T cells increase their expression of IL21R, and an unusually high concentration of IL21R-expressing T cells in joints may indicate joint inflammation and possible arthritis. Illustrative medical conditions that may be diagnosed by the binding proteins of the invention include, but are not limited to, multiple sclerosis, rheumatoid arthritis, and transplant rejection.

Toxicity Studies with Anti-IL21R Binding Proteins

The binding proteins, e.g., antibodies, that act as antagonists can be used to regulate at least one IL21R-mediated immune response; and thus, can be used to treat a variety of immune disorders without having any adverse effects on the immune system, e.g., without delivering activating signals to the immune system (e.g., the human immune system), activating peripheral blood mononuclear cells (PBMCs), and inducing cytokine storm in subjects. Moreover, the binding proteins of the present invention do not induce activation of the IL21 pathway in subjects.

As illustrated in the Examples, AbS and several other anti-IL21R binding proteins act as anti-IL21R antagonistic binding proteins, but do not induce any of the toxic events associated with cytokine storm. Thus, in some embodiments, the present invention also provides a method of determining or predicting whether an antagonist, e.g., an antagonistic anti-IL21R binding protein, may have adverse effects in clinical trials and therapy, e.g., activation of cytokine storm.

In some embodiments, the method may be an in vitro method. In one embodiment of the invention, the method can be used to detect, e.g., the activating effects of IL21 and the inhibitory effects of IL21 antagonists, e.g., AbS or other anti-IL21R binding proteins described herein. For instance, in one embodiment of the invention, the method utilizes blood cells, e.g., PBMCs, from mammalian subjects, e.g., human subjects, to test for upregulation of cytokines associated with a toxic immune response (e.g., activation of cytokine storm). Such an in vitro method comprises the steps of: (a) obtaining a blood sample from a mammalian subject; (b) incubating a therapeutic binding protein, e.g., AbS, with the blood sample, wherein the blood sample is a binding protein-treated blood sample; (c) determining the levels of expression of at least one cytokine storm gene in the binding protein-treated blood sample; and (d) comparing the level of expression of the at least one cytokine storm gene in the binding protein-treated blood sample with the level of expression of the at least one cytokine storm gene in an untreated or negative control-treated sample, wherein a level of expression of the at least one cytokine storm gene in the binding protein-treated blood sample substantially greater than the level of expression of the at least one cytokine storm gene in the untreated or negative control-treated sample indicates (e.g., predicts) that the therapeutic binding protein will induce a cytokine storm in the mammalian subject. On the other hand, if the level of expression of the at least one cytokine storm gene in the binding protein-treated blood sample is not substantially greater than the level of expression of the at least one cytokine storm gene in the untreated or negative control-treated sample, then it may be an indication (e.g., prediction) that the therapeutic binding protein will not induce a cytokine storm in the mammalian subject.

In some embodiments, the in vitro method may be conducted in multi-well plates. For example, the anti-IL21R antagonistic binding proteins or control reagents are either directly coated onto the wells of the plate (dry-coated) or applied to the anti-IgG-coated wells of the plate, and exposed to PBMCs from mammalian donors.

In other embodiments, the method used to determine whether a therapeutic binding protein will induce cytokine storm is an ex vivo whole blood method e.g., a human whole blood method or a monkey whole blood method, that can be used to detect the activating effects of IL21 and the inhibitory effects of IL21 antagonists, e.g., AbS or other antagonistic binding proteins described herein.

Alternatively, the method is an in vivo assay and is used to determine the post-dosing effect of AbS or other binding proteins described herein in a subject. Such post-dosing methods may be conducted after administration of an anti-IL21R antagonistic binding protein, e.g., AbS, to a mammalian subject, e.g., nonhuman mammalian subject (e.g., cynomolgus monkey). For example, in a method to predict whether a therapeutic binding protein will induce a cytokine storm in a first mammalian subject (e.g., a human subject), the method may comprise: (a) administering a therapeutic binding protein, e.g., AbS, to a second mammalian subject (e.g., a cynomolgus monkey subject), wherein the second mammalian subject is a binding protein-treated second mammalian subject; (b) obtaining a blood sample from the binding protein-treated second mammalian subject; (c) determining the level of expression of at least one cytokine storm gene in the blood of the binding protein-treated second mammalian subject; and (d) comparing the level of expression of the at least one cytokine storm gene in the blood of the binding protein-treated second mammalian subject to the level of expression of the at least one cytokine storm gene in the blood of the untreated second mammalian subject, wherein a level of expression of at least one cytokine storm gene in the binding protein-treated second mammalian subject substantially greater than the level of expression of the at least one cytokine storm gene in the untreated second mammalian subject indicates that the therapeutic binding protein will induce cytokine storm in the first mammalian subject. Alternatively, if the level of expression of the at least one cytokine storm gene in the blood of the binding protein-treated second mammalian subject is not substantially greater than the level of expression of that cytokine storm gene in the untreated second mammalian subject, it may indicate (e.g., predict) that the therapeutic binding protein will not induce a cytokine storm in the first mammalian subject.

In one embodiment, the in vivo method comprises administration of a large dose, i.e., a dose larger than the anticipated clinical dose, of the anti-IL21R antagonistic binding protein to, e.g., the cynomolgus monkey, and monitoring whole blood samples for changes in cytokines associated with either or both a toxic immune response (cytokine storm) and an IL21 response. Thus, in some embodiments, the first mammalian subject is a human subject, while the second mammalian subject is a cynomolgus monkey subject. One skilled in the art will understand that the second mammalian subject may be any subject suitable for testing antagonistic binding protein toxicity, e.g., a rodent subject, another nonhuman primate subject.

As used herein, the term “binding protein-treated” refers to a sample or a subject that is treated with the therapeutic binding protein, e.g., a therapeutic antibody, e.g., anti-IL21R antibody (e.g., AbS) to determine the level of upregulation of cytokine storm genes. “Untreated” refers to a sample or a subject to which no activating or inhibiting agent, e.g., binding protein, antibody, or cytokine, is added. Untreated subject or sample is used as a negative control to compare to the level of cytokine upregulation in the binding protein-treated subject. Additionally, “negative control-treated” refers to a sample or a subject that is treated with a negative control binding protein, e.g., IgGTM (IgG1 anti-tetanus triple mutant), IgG1 (IgG1 anti-tetanus wild type), or IgGFc (Fc control) antibody. “Positive control-treated” refers to a sample or a subject that is treated with IL21 cytokine. In some embodiments of the invention, the blood sample may be a whole blood sample, e.g., a human whole blood sample or a cynomolgus monkey whole blood sample. In another embodiment, the blood sample may be a peripheral blood mononuclear cell (PBMC) sample.

In addition to testing for upregulation of cytokine storm genes, the methods of the present invention may simultaneously or otherwise test for upregulation of IL21-responsive cytokines and proteins. The cytokines associated with cytokine storm (i.e., cytokine storm genes), include, but are not limited to, IL4, IL2, IL1β, IL12, TNF, IFNγ, IL6, IL8, and IL10. The IL21-responsive cytokines and proteins include, but are not limited to, CCL19, CCL2, CCL3, CCR2, CD19, CD40, CSF2, CSF3, CXCL10, CXCL11, GZMB, IFNγ, IL10, IL12β, IL1β, IL2RA, IL6, PRF1, PTGS2, and TBX21. Thus, it is evident that some, but not all, cytokines associated with cytokine storm overlap with IL21-responsive cytokines. The methods of the present invention can comprise determining the level of expression of at least one, at least two, at least three, at least four, at least five, at least six, at least seven, at least eight, or at least nine or more cytokine storm genes. In one embodiment, the method of the present invention comprises determining the level of expression of nine cytokine storm genes. Similarly, the methods of the present invention may comprise determining the level of at least one, at least two, at least three, at least four, at least five, at least six, at least seven, at least eight, at least nine, at least ten, at least eleven, at least twelve, at least thirteen, at least fourteen, or at least fifteen, at least sixteen, at least seventeen, at least eighteen, at least nineteen, at least twenty, or at least twenty-one or more IL21-responsive cytokines.

Cytokine changes can be monitored by any of the methods for testing changes in RNA or protein expression. In one embodiment, cytokine changes, e.g., upregulation of cytokines associated with toxic immune response or IL21-responsive cytokines, may be detected by any of the methods for testing changes in gene or protein expression, such as either protein or mRNA detection methods. Upregulation of gene expression may be tested by upregulation of mRNA expression, and may be detected by screening targets by real-time PCR(RT-PCR) on a TAQMAN® Low Density Array. In another embodiment of the invention, upregulation of gene expression may be tested by measuring upregulation of protein expression. In one embodiment, the levels of cytokine may be determined by measuring cytokine release, e.g., by using MSD multiplex immunoassay (Meso Scale Discovery, Gaithersburg, Md.). Specific examples of the assays for testing binding proteins of the invention are described in the Examples.

One skilled in the art will recognize that, in addition to the binding proteins described in the Examples, any binding protein can be used in the assays described herein to determine whether the binding proteins act as antagonists, e.g., IL21R antagonists, without inducing toxicity, including the toxic events associated with cytokine storm.

Another aspect of the present invention relates to kits for predicting whether a therapeutic binding protein will induce a cytokine storm upon administration. For example, the kit may provide a oligonucleotide microarray chip or the like to assess the levels of key genes related to predicting cytokine storm. In other embodiments, other aspects of the present invention may be the focus of kits, and one of skill in the art will be able to construct/formulate such kits and their components based on the present disclosure.

### Combination Therapy

In one embodiment, a pharmaceutical composition comprising at least one anti-IL21R binding protein and at least one therapeutic agent is administered in combination therapy. The therapy is useful for treating pathological conditions or disorders, such as immune and inflammatory disorders. The term “in combination” in this context means that the binding protein composition and the therapeutic agent are given substantially contemporaneously, either simultaneously or sequentially. If given sequentially, at the onset of administration of the second compound, the first of the two compounds may still be detectable at effective concentrations at the site of treatment.

For example, the combination therapy can include at least one anti-IL21R binding protein coformulated with, and/or coadministered with, at least one additional therapeutic agent. The additional agents may include at least one cytokine inhibitor, growth factor inhibitor, immunosuppressant, anti-inflammatory agent, metabolic inhibitor, enzyme inhibitor, cytotoxic agent, and cytostatic agent, as described in more detail below. Such combination therapies may advantageously utilize lower dosages of the administered therapeutic agents, thus avoiding possible toxicities or complications associated with the various monotherapies. Moreover, the therapeutic agents disclosed herein act on pathways that differ from the IL21/IL21R pathway, and thus are expected to enhance and/or synergize with the effects of the anti-IL21R binding proteins. Kits for carrying out the combined administration of anti-IL21R antibodies with other therapeutic agents are also provided. In one embodiment, the kit comprises at least one anti-IL21R antibody formulated in a pharmaceutical carrier, and at least one therapeutic agent, formulated as appropriate in one or more separate pharmaceutical preparations.

The entire contents of all references, patent applications, and patents cited throughout this application are hereby incorporated by reference herein.

### EXAMPLES

The invention will be further illustrated in the following nonlimiting examples. The Examples that follow are set forth to aid in the understanding of the invention but are not intended to, and should not be construed to limit the scope of the invention in any way. The Examples do not include detailed descriptions of conventional methods, e.g., polymerase chain reaction, real-time PCR, cloning, transfection, basic aspects of methods for overexpressing proteins in cell lines, and basic methods for protein purification. Such methods are well known to those of ordinary skill in the art.

### Example 1

### Generation of Anti-IL21R Binding Proteins

The anti-IL21R binding proteins illustrated herein, as well as their utility as therapeutic agents for treating a number of IL21-associated disorders, are described in detail in, e.g., U.S. patent application Ser. No. 12/472,237 (incorporated by reference herein). The sequences of several anti-IL21R binding proteins, as well as other sequences involved in generating and studying these binding proteins (e.g., SEQ ID NOs:196-210 and 230-238), are disclosed in the accompanying Sequence Listing and are described in detail in Table 2B and/or in U.S. patent application Ser. No. 12/472,237, incorporated by reference in its entirety.

### Example 2

### Agonistic Response of Human Whole Blood to IL21 is Neutralized by Ex Vivo Treatment with Anti-IL21R Binding Proteins

To demonstrate the utility of anti-IL21R binding proteins in inhibiting IL21R-dependent responses, the inhibition of agonistic response of human whole blood to IL21 with anti-IL21R binding proteins was analyzed. Human whole blood was drawn by the Human Blood Donor Program in Cambridge, Mass. All human blood samples were collected in BD Vacutainer™ CPT™ cell preparation tubes. Collection tubes contained sodium heparin. Samples were maintained at ambient temperature and processed immediately. Blood was divided into 1 to 2 mL aliquots in cryovials, and treated with IL21, AbS, or control proteins. When samples were treated with both anti-IL21 binding protein and IL21, the binding protein was added immediately prior to IL21. Samples were then incubated at 37° C. in a Form a Scientific Reach-In Incubator Model # 3956 for four hr while mixed continuously at 15 RPM using the Appropriate Technical Resources Inc (ATR) Rotamix (Cat. # RKVS) rotating mixer (serial #0995-52 and #0695-36), or using the Labquake® Tube Shaker/Rotator (Cat. # 400110) during the incubation. Aliquots (0.5 mL) were removed using a Gilson P1000 pipette with ART 1000E tips (Cat. # 72830-042) and added to 2.0 mL microtubes (Axygen Scientific, Cat. # 10011-744) containing 1.3 mL of RNAlater® supplied with the Human RiboPure™-Blood Kit (Ambion, Austin, Tex.; Cat. # AM1928) and mixed thoroughly by five complete inversions. Samples were stored at ambient temperature overnight and then frozen at −80° C. pending RNA purification.

RNA was isolated using the Human RiboPure™ Blood Protocol (Ambion, Cat. # AM1928). The Human RiboPure™ RNA isolation procedure consists of cell lysis in a guanidinium-based solution and initial purification of the RNA by phenol/chloroform extraction, and final RNA purification by solid-phase extraction on a glass-fiber filter. The residual genomic DNA was removed according to the manufacturer's instructions for DNAse treatment using the DNA-free™ reagents provided in the kit. For all samples, RNA quantity was determined by absorbance at 260 nm with a NanoDrop 1000 (NanoDrop, Wilmington, Del.). RNA quality was spot-checked using a 2100 Bioanalyzer (Agilent, Palo Alto, Calif.). Samples were stored at −80° C. until cDNA synthesis was performed.

According to the manufacturer's instructions, cDNA was reverse transcribed from total RNA using a High Capacity cDNA Reverse Transcription Kit (ABI, Cat. # 4368814) with additional RNase inhibitor at 50 U/sample (ABI, Cat. # N808-0119). cDNA samples were stored at −20° C. until RT-PCR (real-time PCR) was performed. The amount of cDNA loaded on a Taqman® Low Density Array card (TLDA) was determined using the lowest RNA yield obtained within an experiment.

TLDAs are microfluidic cards comprised of Applied Biosystem's Assays-on-Demand (AOD) gene-specific primer pair/probe sets. Each well contains a single AOD comprised of gene-specific unlabeled forward and reverse primers and a gene-specific 5′ FAM™ dye-labeled Taqman minor groove binder (MGB) probe with a nonfluorescent quencher (NFQ). These AODs are prevalidated, quality-control tested, and optimized for use on any ABI PRISM sequence detection system.

Sample cDNA was mixed with a Taqman® Universal PCR Master Mix (Applied Biosystems; Cat. # 430-4437) and added onto the TLDA. TLDAs were then spun at 1200×g at RT for two consecutive 1 min spins, sealed, and loaded into the ABI 7900HT Sequence detector (Sequence Detector Software 2.2.3, Applied Biosystems). The following universal thermal cycling conditions (50° C. for 2 min, 95° C. for 10 min, 40 cycles of 95° C. for 15 sec, and 60° C. for 1 min) were used for all TLDAs described in this and the following examples. These universal thermal cycling conditions were used for all subsequent experiments.

Endogenous controls were used to normalize sample quantification by accounting for variations in concentrations of samples loaded. Relative quantification for all TLDA data was done in a Spotfire-guided application (Livak and Schmittgen (2001) Methods 25:402-08).

To check for ex vivo effects of IL21, experiments were conducted to test whether human whole blood and/or purified PBMCs responded to IL21 with detectable changes in gene expression levels. Whole blood or purified PBMCs from human donors were incubated in the presence and absence of IL21, and RNA levels were determined using TLDA cards. Two different TLDAs were used to measure RNA expression levels. The first, Human Immune TLDA (ABI, Catalog #4370573), tested 96 genes, of which 91 were detectable in stimulated human blood. PBMCs stimulated with LPS or PHA from human donor whole blood were used as positive control. To test the upregulation of IL21R in response to IL21 stimulation, results were obtained using a custom designed TLDA that contained the IL21R gene.

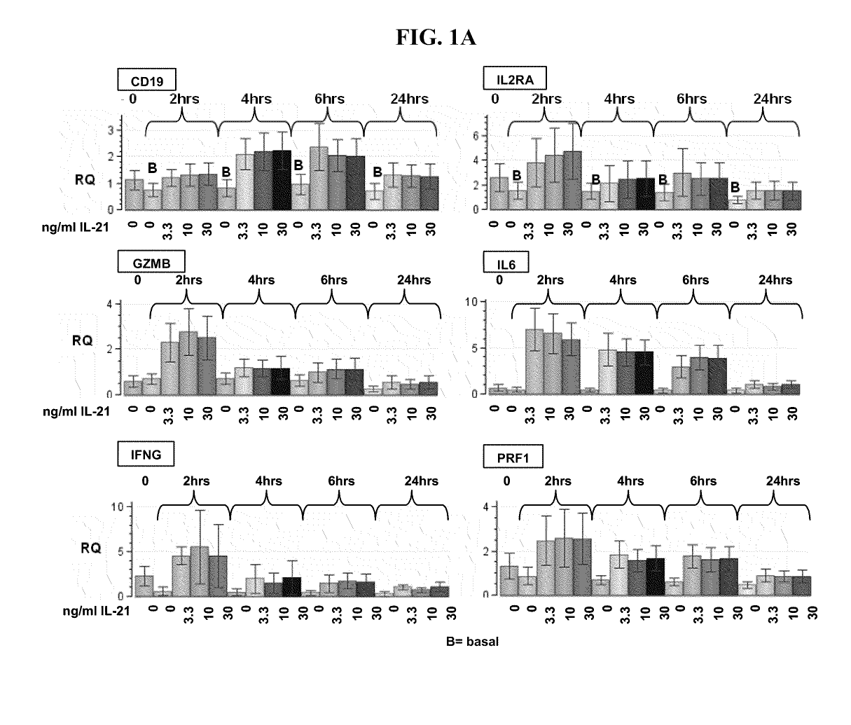

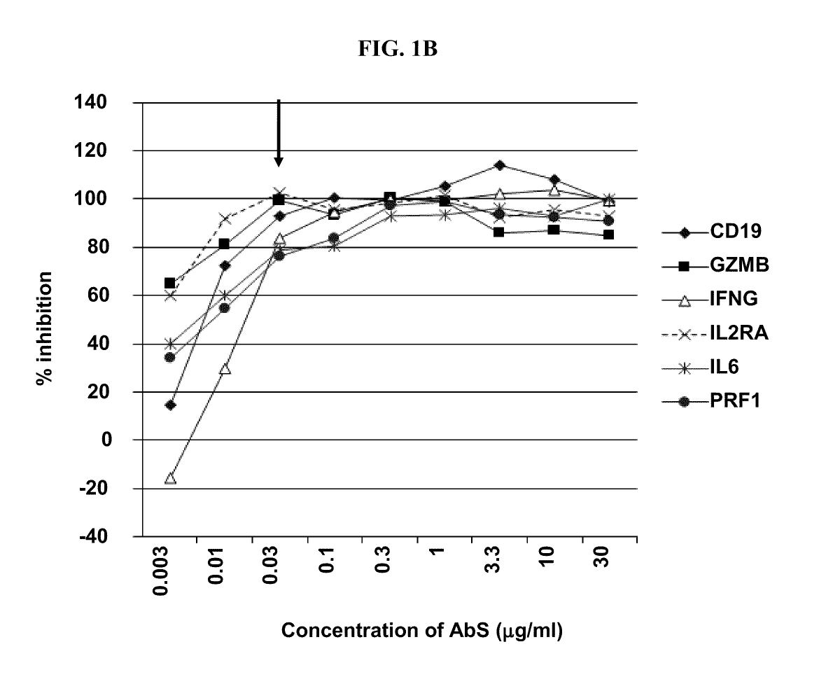

In order to determine optimal time and dose of IL21 treatment for generation of maximal signal, whole blood samples from five healthy donors were incubated in the presence of 3.3, 10 or 30 ng/ml of IL21 for 2, 4, 6 or 24 hr. RNA was isolated and gene expression levels measured. Significant and robust IL21 dependent signals were obtained for six genes: IL6, IFNγ, IL2RA, GZMB, PRF1, CD19. The optimal signal for all but CD19 was obtained at 2 hr (FIG. 1A). There was little difference in the response obtained at 3.3, 10 or 30 ng/ml IL21. Response to ex vivo IL21 treatment was consistent between all five donors (data not shown). Based on the results obtained with these five donors, the assay conditions chosen to titrate the inhibitory effect of AbS on the ex vivo response to IL21 were: two-hr stimulation with 10 ng/ml of IL21.

To determine the dose of AbS to optimally block the effect of IL21, samples from four individual donors were preincubated for 2 hr at the indicated concentrations of AbS and IgG₁TM, both diluted in PBS, before the addition of 10 ng/ml of IL21. Following the addition of IL21, samples were incubated for an additional 2 hr. Addition of 0.1 μg/mL AbS resulted in full inhibition, so 0.003 μg/mL of AbS was used for subsequent experiments. AbS, but not IgG₁TM, inhibited the response of all six genes tested in all four donors, as demonstrated in FIG. 1B.

These results demonstrate the utility of anti-IL21R binding proteins in inhibiting IL21-dependent responses and define methods for measuring the response to IL21 in human blood.

### Example 3

### Evaluation of Potential for Cell Signaling and Cytokine Storm after Anti-IL21R Binding Protein Treatment in Human and Cynomolgus Monkey Subjects

### Example 3.1

### Measurement of In Vitro Activation of Cytokines by IL21 Ligand in Human Peripheral Blood Mononuclear Cells (PBMCs)

Following the recent failure of TGN1412 (the anti-CD28 antibody) in clinical trials due to the induction of cytokine storm, which resulted in systemic inflammatory response and multiorgan failure, it became imperative to test lead therapeutic binding proteins for induction of similar toxic responses. Subsequent to the TGN1412 clinical trials, in vitro activating protocols were developed to test the activation of PBMCs by TGN1412 cross-linked to the surface of plastic tissue culture wells (Stebbings et al. (2007) J. Immunol. 179:3325-31). Six different protocols were tested for activation of PBMCs by TGN1412, and three were shown to induce activation (Stebbings et al. (2007) supra). Of these protocols, two (presentation on anti-IgG, and dry coating) were tested herein. IL21 is known to induce several cytokine storm-related genes under specific conditions and from different cell lines and purified cell populations, but the extent of IL21-induced activation on PBMCs and whole blood was unknown. Thus, induction by IL21 of 12 proteins and 90 mRNAs associated with immune activation was tested.

Fresh human PBMCs were isolated from the whole blood of five healthy donors using sodium citrate CPT Vacutainer tubes (BD, Franklin Lakes, N.J.). Approximately 310-450 ml of whole blood (8 ml/tube) from each donor was purchased from Research Blood Components (Brighton, Mass.) and extracted on different days. Each sample was processed within 4 hr of draw. CPT tube aliquots (8 ml) were spun at 1500×g for 20 min at room temperature (to remove plasma, red blood cells, neutrophils, etc.). PBMCs were washed in PBS twice (pH=7.2), and post-purification differential cell counts were taken using a Pentra 60C (HORIBA ABX Diagnostics, Irvine, Calif.). Final cell pellet was reconstituted in cell culture media (RPMI-1640, 10% HIFBS, 2 nM L-glutamine, 100 unit/ml penicillin and 100 mg/ml streptomycin, 10 mM Hepes (1:100), 1 mM sodium pyruvate, 50 μM β-mercaptoethanol, 12.5 ml/L of 20% glucose) to a final concentration of 2-2.5×10⁶/ml. 100 μL/well suspension cells were added to wells in which titrated IL21 was also added.

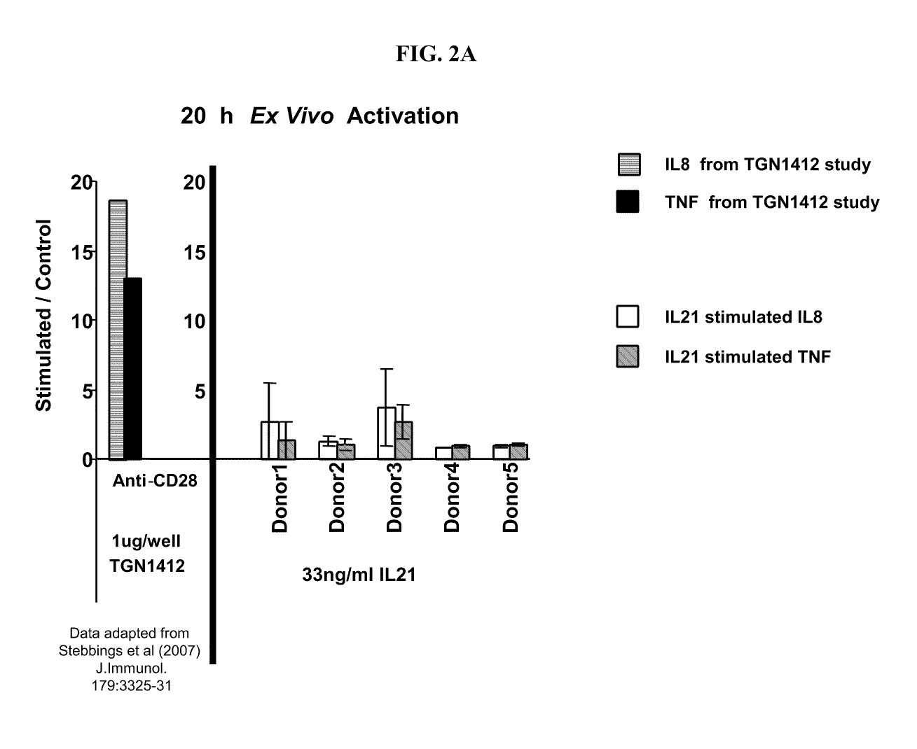

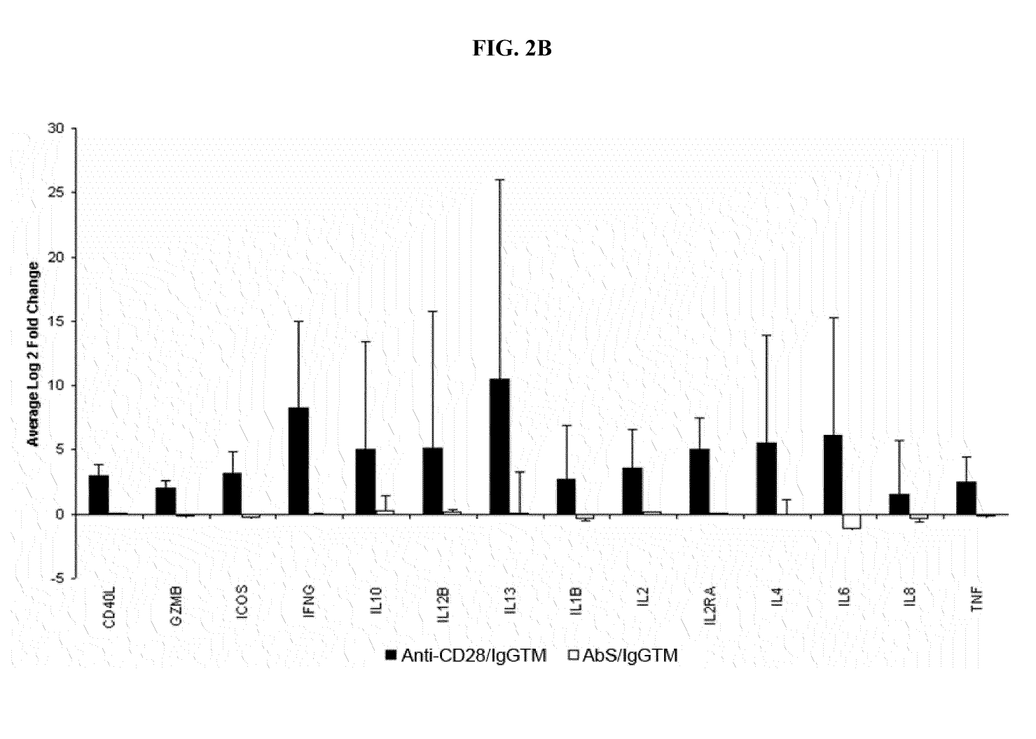

To test the magnitude of protein induction by IL21 as compared to TGN1412, 33 ng/ml of IL21 was incubated in 96-well plates with PBMCs from five individual human subjects. MSD multiplex immunoassay plates (Meso Scale Discovery, Gaithersburg, Md.) were used to measure secreted cytokine levels in harvested cell-conditioned media from PBMC cultures according to the manufacturer's instructions. The results were compared to the reported signal for cross-linked TGN1412 at 1 μg/well (Stebbings et al. (2007) supra). The magnitude of the in vitro IL8 or TNFα protein signal induced by either TGN1412 or IL21 after 20 h incubation is shown in FIG. 2A. According to Stebbings et al., IL8 and TNFα were induced 18- and 13-fold, respectively, by TGN1412 stimulation, whereas much less induction of IL8 and TNFα was demonstrated for IL21 (1.5- to 4-fold increase).

### Example 3.2

### Comparison of Effects of Cross-linked Anti-CD28 and Cross-linked AbS

PBMCs from a total of 15 healthy donors were incubated and tested for effects of cross-linked AbS on protein and RNA expression at a variety of time points, IgG concentrations, and cross-linking protocols.

At the end of the incubation, all 96-well plates were spun at 280×g (in cold) using a Jouan CR422 refrigerated centrifuge (Jouan Inc., Winchester, Va.). RNA extraction from cell pellets began with the addition of 100 μL of RLT lysis buffer (Qiagen, Valencia, Calif.) containing 1% β-mercaptoethanol to wells, upon removal of conditioned media. The wells were then snap frozen for RNA purification at a later time. Briefly, cell pellets frozen in the RLT lysis buffer were thawed and processed for total RNA isolation using the QIA shredder kit and RNeasy mini-kit (Qiagen) according to the manufacturer's recommendations. All of the samples were subjected to DNase (on-column treatment) to remove potential DNA contamination, and then purified using the columns provided in the Qiagen kit. A phenol-chloroform extraction was then performed, and the RNA was further purified using the RNeasy mini-kit reagents. Eluted RNA was quantified using a NanoDrop ND-1000 spectrophotometer (Thermo Scientific, Wilmington, Del.). Approximately 225 ng of total RNA per sample (per TLDA, see below) was converted to cDNA with the Applied Biosystems High Capacity cDNA Archive kit (Cat. # 4322171; Applied Biosystems, Foster City, Calif.).

For all gene transcription analyses in this and the following studies in Example 3 (human), either the TLDA Human Immune Array cards (Cat. # 4370573; TAQMAN® Low Density Array, Applied Biosystems) or a custom TAQMAN® Low Density Array from Applied Biosystems and designed to query the known IL21-responsive and cytokine storm-associated genes, was used.

The results obtained with the five donors tested at 10 μg/well of cross-linked antibodies are shown in FIG. 2B. The results confirmed that the anti-CD28 cross-linking conditions described by Stebbings et al. (supra) induced robust secretion of cytokine storm-associated cytokines. In addition, and as expected, large increases were observed in RNA expression levels of 14 genes selected on the basis of known association with cytokine storm and/or association with IL21-mediated activation (FIG. 2B; filled bars). In contrast, cross-linked AbS did not induce increases in RNA expression (FIG. 2B; open bars).

The levels of some cytokines observed with control IgG₁TM were increased over the levels in media control groups, although, as shown in FIG. 2B, levels in anti-CD28-stimulated groups were significantly higher than levels in control IgG₁TM-stimulated cultures. To examine whether the observed IgG₁TM effects were attributable to characteristics specific to that particular reagent, two other cross-linked Ig control reagents were tested. Both of these reagents—human IgG₁ wildtype, which shares all characteristics with IgG₁TM except 3 mutations in the constant region, and purified human-Fc—induced similar increases over media control (data not shown). These results show that IgG reagents induce activation under the cross-linking protocols employed in these studies and underscore the need for well-characterized control IgG reagents in such studies.

### Example 3.3

### Detection of Human PBMC Activation with In Vitro Cross-Linked Anti-IL21R Binding Protein

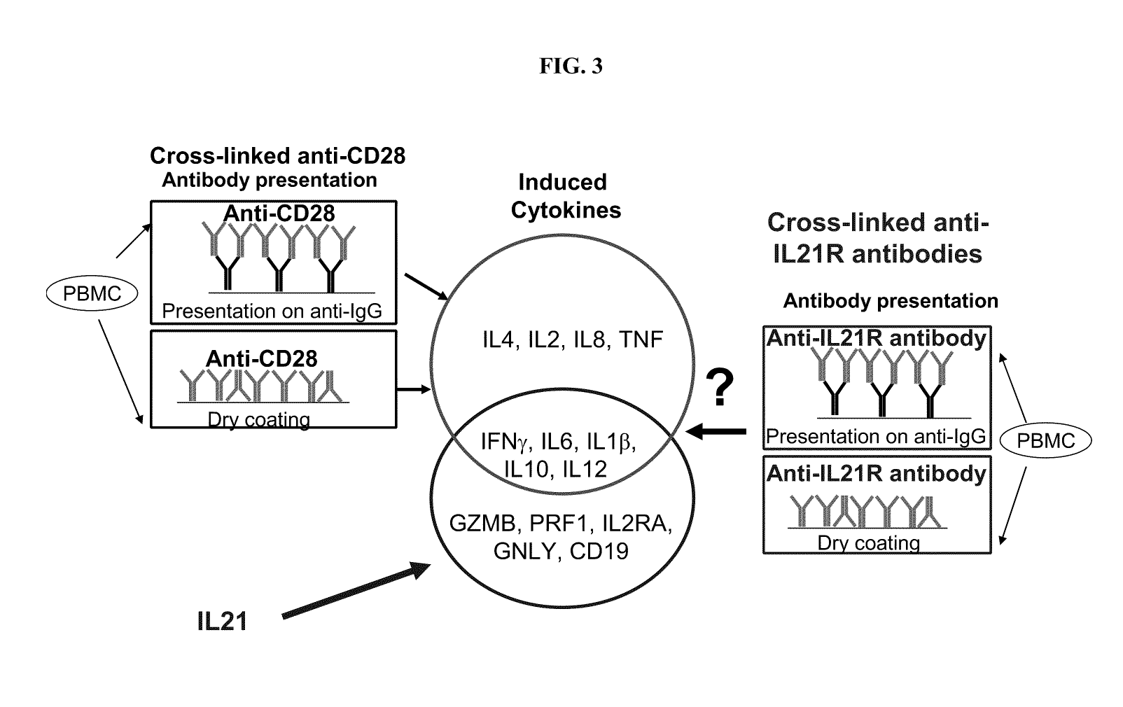

In order to determine whether anti-IL21R binding proteins induced similar signals to those observed with IL21, or signals associated with cytokine storm, in vitro tests of cross-linked binding proteins (e.g., AbS) on PBMCs from fifteen individual human donors were performed (FIG. 3). Specifically, binding proteins (at 100 ng, 300 ng, 1 μg, or 10 μg per well) or control IgGs [IgGTM, IgG1 (human IgG anti-tetanus wild type), or IgGFc] were adsorbed onto either anti-IgG coated or dry-coated wells of a 96-well plate. IL21 and anti-CD28 (ANC28.1/5D10; Ancell, Bayport, Minn.)) were used as positive controls for detection of activation signal.

In the dry-coated protocol, binding proteins were coated onto wells by air drying a master stock solution of each of the titrated binding proteins in sterile PBS (pH=7.2) in a total volume of 50 μl per well, which was applied directly onto wells of 96-well polystyrene Corning high-bind plates (Cat. # 3361; Corning, Lowell, Mass.). These plates were left open under a tissue culture hood at RT overnight for drying.

In the anti-IgG-coated protocol, a master stock solution of 100 μl per well of titrated binding proteins in sterile PBS (pH=7.2) was applied directly onto wells of the 96-well goat anti-human IgG plate (H+ L) (Cat. # 354180; BD Biosciences, Bedford Mass.) at RT for 1 h, and then agitated overnight at 4° C.

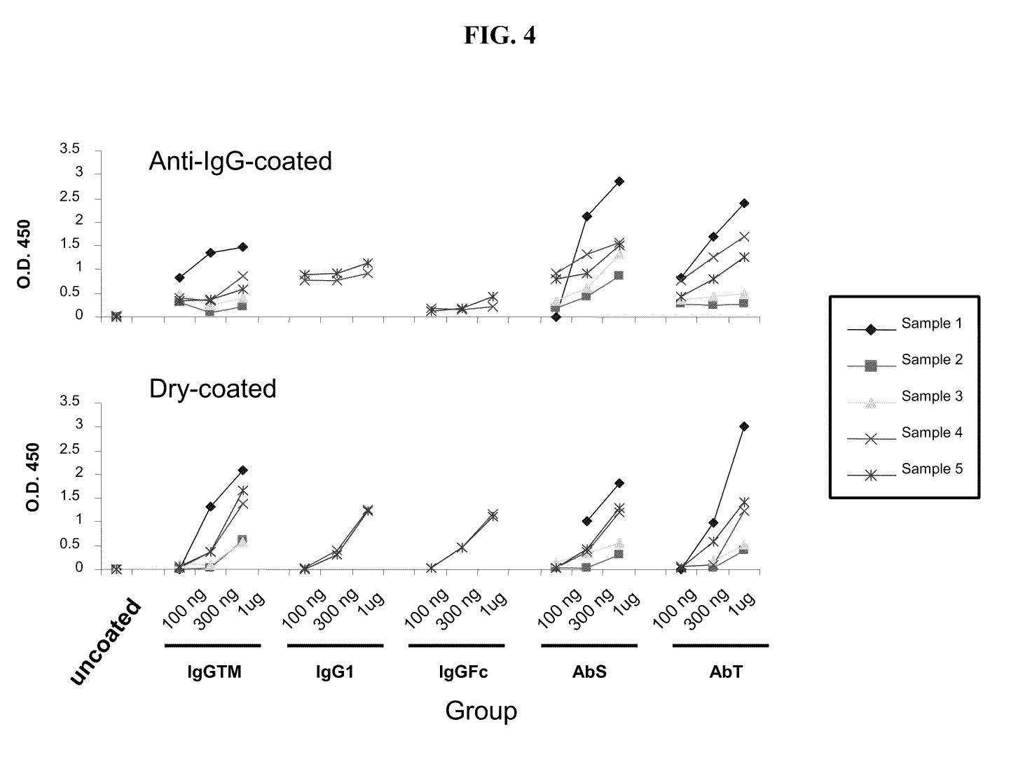

Both the dry-coated and anti-IgG-coated protocols resulted in well-bound human IgGs for PBMC cross-linking experiments (FIG. 4). The persistence of the coated binding protein in the culture wells was confirmed for each condition by ELISA detection of human IgG after the cell culture samples were collected. Wells were washed 4× with 200 μl/well of 0.03% Tween-20 in PBS. The detection antibody, mouse anti-human IgG (Fc) HRP (Cat. # 9040-05; Southern Biotech, Birmingham, Ala.) was diluted at a ratio of 1:2000 in assay buffer (0.5% BSA+0.02% Tween-20 in PBS), and 100 μl added to each well and agitated slowly for 30 min. Wells were then washed 4× with 200 μL/well of 0.03% Tween-20 in PBS. Finally, 100 μl/well of BioFX TMB HRP Microwell Substrate (BioFX Laboratories, Inc., Owings Mills, Md.; Cat. # TMBW-0100-01) was added into each well to allow color development for 8 min at RT. The reaction was stopped by 50 μl/well of 0.18N H₂SO₄. The relative amount of bound binding protein was recorded using a Spectra Max Plus plate reader (Molecular Devices, Sunnyvale, Calif.) by measuring the absorbance at O.D. 450 nm.

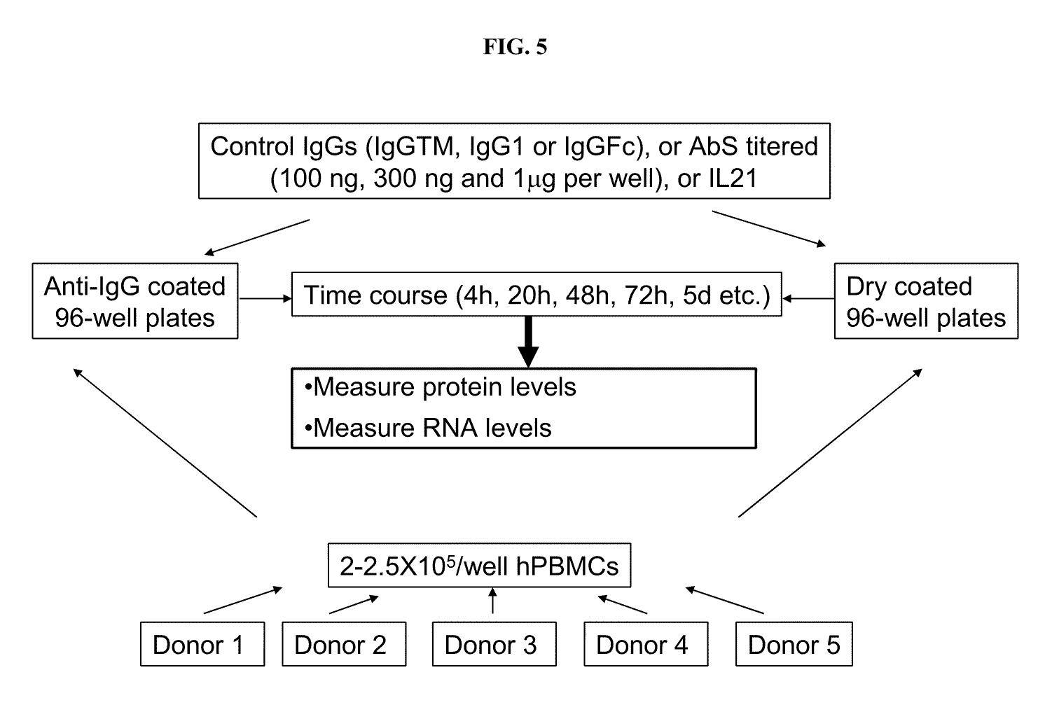

Following adsorption of the binding proteins, plates were incubated with 2-2.5×10⁵ cells/well of human PBMC, which were isolated as described in Example 3.1, for a period of 4, 20, 48, 72, or 120 hr, and protein and RNA levels were measured (FIG. 5). Table 3 shows the results of the protein and RNA levels tested on the first five human donors. Samples from the subsequent ten donors were tested using a custom TLDA containing the following genes: 21 test genes (CXCL10, ICOS, IFNγ, IL2RA, CD19, PRF1, GZMB, GNLY, IL13, IL17, CXCL11, CD40LG, IL1b, IL2, IL4, IL6, IL8, IL10, IL12B, TNF, and IL21R) and three endogenous control genes (18S, ZNF592, and PTPRC).

TABLE 3
Protein or RNA Tested for AbS-Mediated Induction

| | | | | | |
|---|---|---|---|---|---|
| 18S | CCR7 | CSF3 | HLA | IL2 | PTGS2 |
| ACE | CD19 | CTLA4 | HLA | IL2RA | PTPRC |
| ACTB | CD28 | CXCL10 | HMOX1 | IL3 | REN |
| AGTR1 | CD34 | CXCL11 | ICAM1 | IL4 | RPL3L |
| AGTR2 | CD38 | CXCR3 | ICOS | IL5 | SELE |
| BAX | CD3E | CYP1A2 | IFNγ | IL6 | SELP |
| BCL2 | CD40 | CYP7A1 | IKBKB | IL7 | SKI |
| BCL2L1 | CD40LG | ECE1 | IL10 | IL8 | SMAD3 |
| C3 | CD4 | EDN1 | IL12A | IL9 | SMAD7 |
| CCL19 | CD68 | FAS | IL12B | LRP2 | STAT3 |
| CCL2 | CD80 | FASLG | IL13 | LTA | TBX21 |
| CCL3 | CD86 | FN1 | IL15 | MYH6 | TFRC |
| CCL5 | CD8A | GAPDH | IL17 | NFKB2 | TGFB1 |
| CCR2 | COL4A5 | GNLY | IL18 | NOS2A | TNF |
| CCR4 | CSF1 | GUSB | IL1A | PGK1 | TNFRSF18 |
| CCR5 | CSF2 | GZMB | IL1B | PRF1 | VEGF |

The gene transcript levels for the genes shown above were assayed using the human immune array TLDA card. Cytokines underlined (CCL3, IFNγ, IL10, IL12B, IL13, IL1β, IL2, IL4, IL5, IL6, IL8 and TNF) were also measured at the protein level by MSD multiplex-immunoassay.

The protein levels were determined by multiplex-immunoassay for Table 3. Specifically, 6-well, 10 spot (IFNγ, IL1β, IL2, IL4, IL5, IL8, IL10, IL12p70, IL13, TNF) MSD plates (MS6000 Human TH1/TH2 10-Plex Kit, Meso Scale Discovery) and 96-well customized 2 spot (IL6 and CCL3) MSD plates (Meso Scale Discovery) were used to measure secreted cytokine levels in harvested cell condition media from PBMC cultures, according to the manufacturer's instructions. The sensitivity of the assays was within the limits of the manufacturer's guidelines.

The RNA levels were determined by screening targets on Human Immune Taqman® Low Density Array, as described in Example 3.2. The RQ of AbS versus IgGTM was a representative of the relative fold-change of anti-IL21R binding protein over control binding proteins at the same concentrations.

Measurements were taken at multiple binding protein concentrations and three different negative control IgGs at multiple time points. IL21 stimulation/anti-CD28 stimulation was included as positive controls, and binding of binding protein to the plate was always confirmed by ELISA.

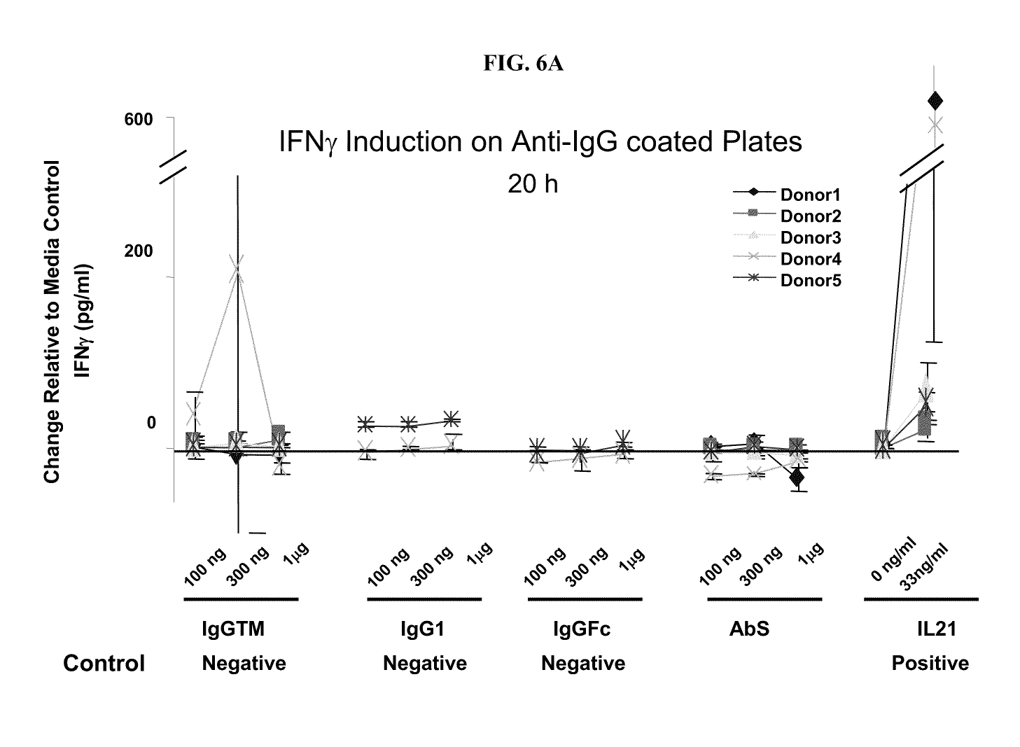

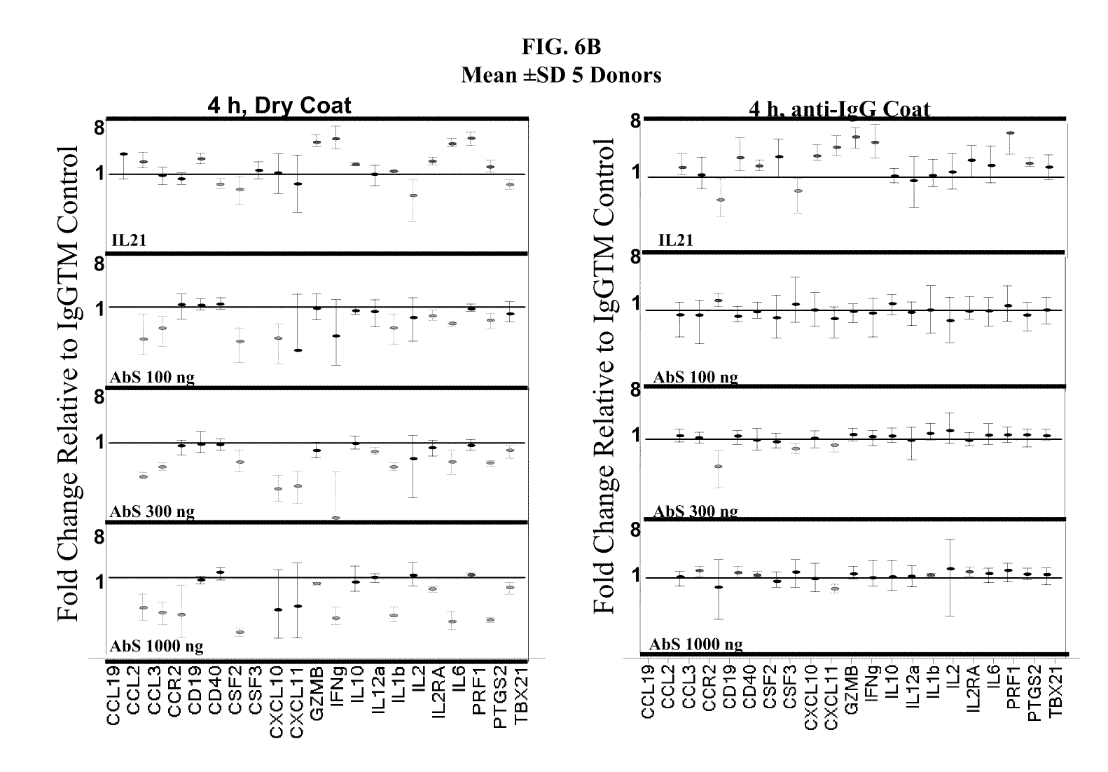

No significant cytokine protein release was demonstrated with cross-linked AbS for all 12 cytokines at the 20-hr time point, as demonstrated by the determination of IFNγ release with binding protein treatment (FIG. 6A). Similarly, cross-linked AbS did not significantly activate human PBMC RNA expression of either IL21-responsive or cytokine storm genes, as demonstrated by either dry-coat or anti-IgG-coat presentation method at the 4-hr time point (FIG. 6B). In fact, none of the IL21-dependent increases were observed with cross-linked AbS relative to IgGTM control.

Thus, AbS does not induce signals observed with IL21 or signals associated with cytokine storm in an in vitro assay of human PBMCs.

In order to control for the inherent variability in treatment response between different donors and to guard against the possibility that any agonistic response induced in a given donor was statistically masked by the lack of response in the other donors, induction gene transcripts due to AbS treatment were compared to the range seen over all donors with control IgGTM. The inherent variability range of the assay was defined as the average of IgGTM control values from all donors +/−3 standard deviations. An activation signal was defined as any value that fell above the inherent variability range of the assay.

Cytokine storm induction values obtained with AbS (at 10, 1, 0.3, and 0.1 μg/well) were compared to the inherent variability range of the assay as defined by values obtained with IgGTM. At 10 μg/well of AbS (the optimal dose for cytokine storm induction by anti-CD28 antibody), no signal was observed for any cytokine storm gene in any donor. The IL2RA value at 0.3 μg/well in one donor was increased 3.18 fold and exceeded the inherent variability range; while the IL2RA value at 0.1 and 0.3 μg/well in another donor was decreased 0.5 and 0.04 fold, respectively, and also exceeded the inherent variability range. However, the IL2RA gene has not been associated with cytokine storm or proinflammatory cascade.

Cytokine storm activation signals for several other binding proteins, including AbV, AbW and AbU, were also determined (data not shown). When individual donors were assessed for any activation signals, a very small number of sporadic signals were observed. For AbV, no activation signal was observed in any donors for any genes at any concentrations tested. For AbW and AbU, a few sporadic activation signals above control were observed in a very small minority of samples, but these signals were at lower concentrations tested.

### Example 3.4

### Agonistic Response of Cynomolgus Monkey Whole Blood to IL21 is Neutralized by Ex Vivo Treatment with Anti-IL21R Binding Proteins

To support the use of cynomolgus monkeys in toxicity studies with antagonistic binding proteins, e.g., AbS, it was necessary to show that AbS induces the desired ex vivo effect of blocking of IL21-induced activation signals in cynomolgus blood.

Cynomolgus whole blood samples were collected in BD Vacutainer™ CPT™ cell preparation tubes. Collection tubes contained one of the following anticoagulants: sodium citrate, lithium heparin, or sodium heparin. Cynomolgus whole blood was drawn and processed immediately. Blood was divided into 1-2 ml aliquots in cryovials, treated with IL21, AbS, or control proteins where indicated. When samples were treated with both binding protein and IL21, the binding protein was added immediately prior to IL21. Samples were then incubated at 37° C. in a Form a Scientific Reach-In Incubator Model # 3956 (Form a Scientific, Inc., Marietta, Ohio) for 4 h while mixed continuously at 15 RPM using the ATR Rotamix rotating mixer (Cat. # RKVS; serial #0995-52 and #0695-36; Appropriate Technical Resources, Inc., Laurel, Md.), or using the LabQuake® Tube Shaker/Rotator (Cat. # 400110; ThermoFischer Scientific, Inc., Dubuque, Iowa) during the incubation. Aliquots (0.5 ml) were removed using a Gilson P1000 pipette with ART 1000E tips (Cat. # 72830-042) and added to 2.0 ml microtubes (Cat. # 10011-744; Axygen Scientific, Union City, Calif.) containing 1.3 ml of RNAlater® supplied with the Human RiboPure™-Blood Kit (Cat. # AM1928; Ambion, Austin, Tex.) and mixed thoroughly by five complete inversions. Samples were stored at ambient temperature overnight and then frozen at −80° C. pending RNA purification.

RNA was isolated using the Human RiboPure™-Blood Protocol (Ambion; Cat. # AM1928). The Human RiboPure™ RNA isolation procedure consists of cell lysis in a guanidinium-based solution and initial purification of the RNA by phenol/chloroform extraction, and final RNA purification by solid-phase extraction on a glass-fiber filter. The residual genomic DNA was removed according to the manufacturer's instructions for DNAse treatment using the DNA-free™ reagents provided in the kit. For all samples, RNA quantity was determined by absorbance at 260 nm with a NanoDrop 1000 (Thermo Scientific). RNA quality was spot-checked using a 2100 Bioanalyzer (Agilent, Palo Alto, Calif.). Samples were stored at −80° C. until cDNA synthesis was performed.

As performed according to the manufacturer's instructions, cDNA was reverse transcribed from total RNA using a High Capacity cDNA Reverse Transcription Kit (Cat. # 4368814; Applied Biosystems Inc., Foster City, Calif.) with additional RNase inhibitor at 50 U/sample (Applied Biosystems Inc.; Cat. # N808-0119). cDNA samples were stored at −20° C. until RT-PCR (real-time PCR) was performed. cDNA samples were assayed using a custom TLDA designed for monkey studies on an ABI PRISM 7900 Sequence detector (Sequence Detector Software v2.2.2, Applied Biosystems) using universal thermal cycling conditions of 50° C. for 2 min, 95° C. for 10 min, then 40 cycles of 95° C. for 15 sec and 60° C. for 1 min.

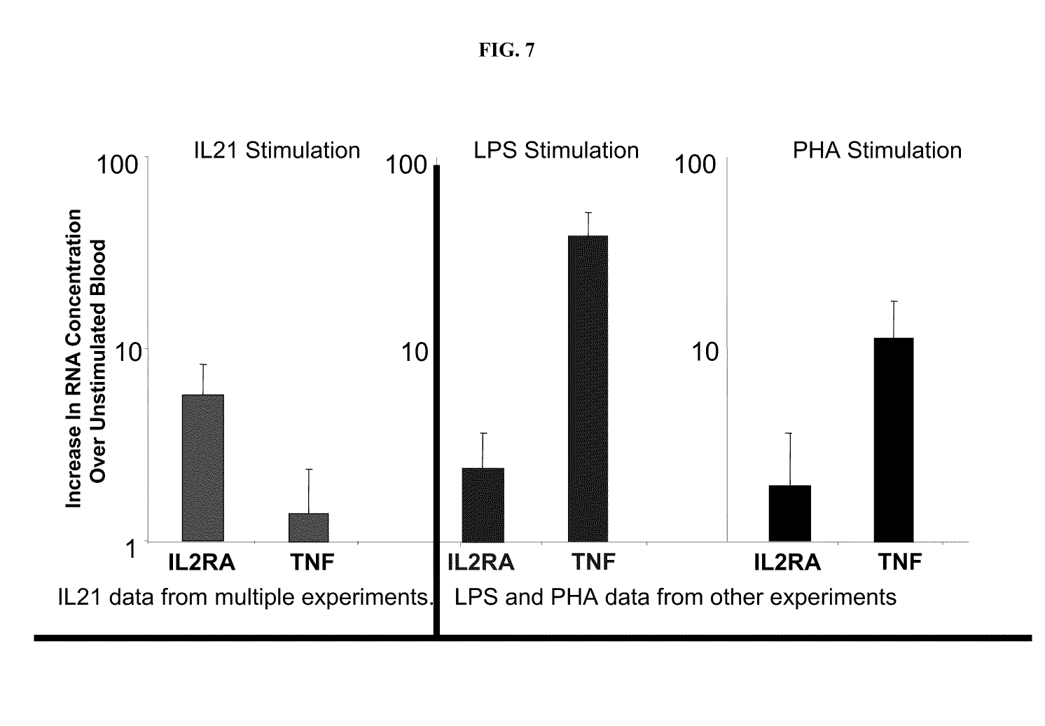

To determine whether IL21 induced similar responses in cynomolgus monkey and human blood, IL21-dependent induction of seventeen RNAs, including PRF1, IL21R, GZMB, IL10, TNF, and IL2RA, was tested. Robust, significant responses to IL21 were observed for several genes, including IL2RA, PRF1, GZMB, and IL21R (data not shown). IL21 induced a robust IL2RA response in cynomolgus monkey blood, but the TNF response was much weaker compared to LPS- and PHA-induced responses observed in separate experiments (FIG. 7).

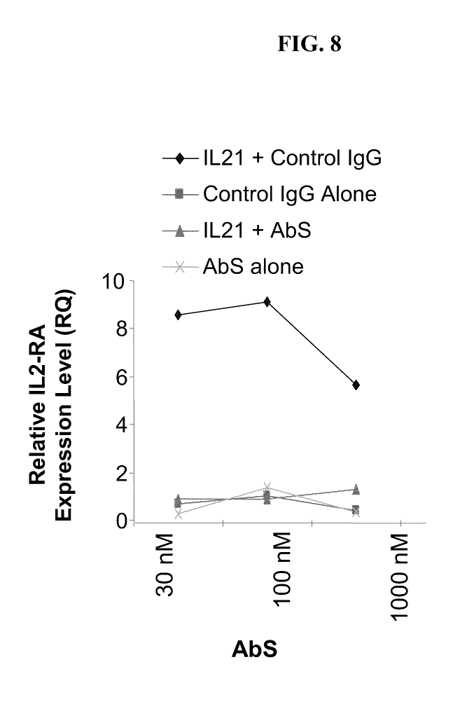

Similar to its response in human blood, AbS inhibited ex vivo response of cynomolgus monkey blood to IL21. Expression levels of eleven cytokines typically induced by IL21 were tested (data not shown). As demonstrated by AbS inhibition of IL2RA (FIG. 8), AbS inhibited the ex vivo response of cynomolgus blood to IL21. These data indicate that AbS has the desired biological activity in cynomolgus monkeys; therefore, cynomolgus monkeys were used for further toxicity studies.

### Example 3.5

### Establishment of In Vivo Nonhuman Model to Test for Binding Protein-Induced Activation of the IL21 Pathway and Cytokine Storm

To demonstrate the in vivo effect of AbS on the IL21 pathway and cytokine storm genes in cynomolgus monkeys, monkeys were divided into two treatment groups—AbS-treated or untreated. Treated animals received a single 100 mg/kg i.v. dose (which is at least 10-fold higher than the anticipated clinical dose) of AbS. Blood was obtained from monkeys at 6 h, 24 h, 14 days, or 56 days post-treatment. Upon removal of the blood from the animal, 1 ml of blood was added immediately to 125 μl of sodium citrate (0.1 M), inverted five times, and then spun at 1200×g for 10 min in a centrifuge. The plasma was aliquoted into a cryotube, and 300 μl of RPMI 1640 was added to the remaining blood pellet (to make up for the loss of plasma). Next, 2.6 ml of RNA later (Ambion; Cat. # AM7020) was added to the blood and medium mixture, mixed well, and frozen at −80° C.

RNA was purified using the RiboPure-Blood Procedure (Ambion; Cat. # AM1928) and quantified using Nanodrop products (Thermo Scientific) monitoring A260/280 OD values, as described in Example 3.4 The quality of each RNA sample was assessed by capillary electrophoresis alongside an RNA molecular weight ladder on the Agilent 2100 bioanalyzer (Agilent Technologies, Palo Alto, Calif.).

RNA from each sample was converted to cDNA with the Applied Biosystems High Capacity cDNA Archive kit (Applied Biosystems Inc., Foster City, Calif.; Cat. # 4322171), loaded onto TLDA cards, and processed as described in the above Examples.

The expression levels of several cytokine storm and IL21-responsive genes were measured, including: TNF, IFNγ, IL6, IL8, IL2, IL12β, IL10, IL2RA, IL21R, PRF1, GZMB, STAT3, TBX21, CSF1, and CD19.

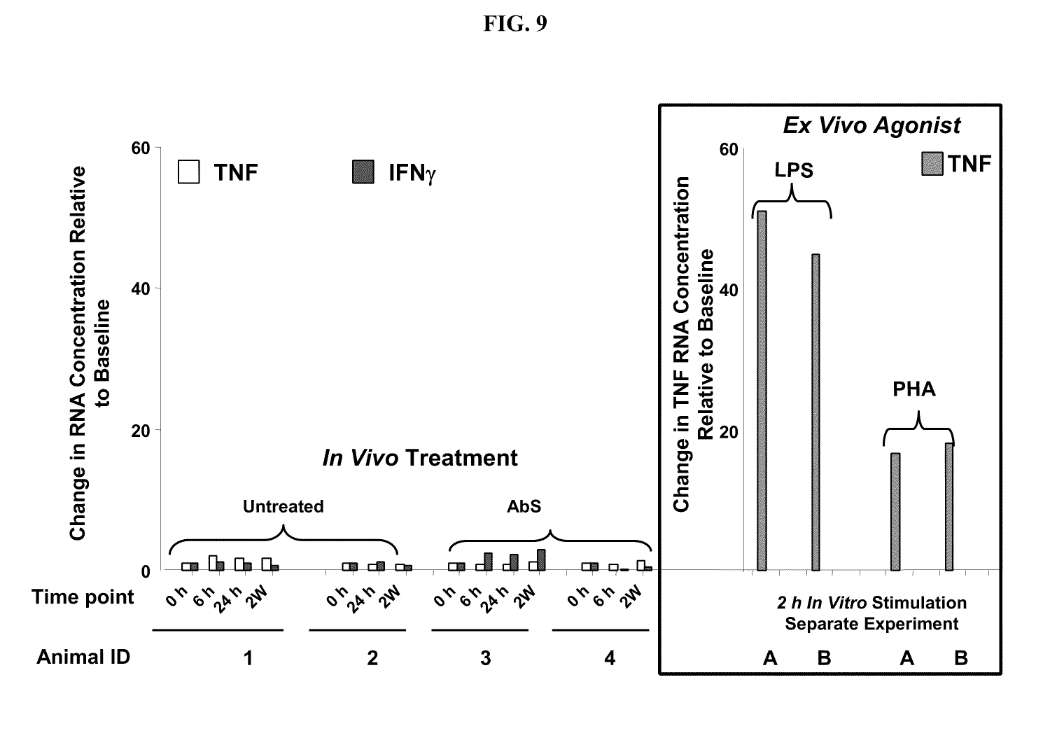

As demonstrated by the effect on TNF and IFNγ (FIG. 9), AbS-treated and control-treated monkeys displayed comparable blood RNA expression levels of IL21-induced and cytokine storm-related genes. In comparison, in vitro agonists LPS and PHA induced 50- and 20-fold stimulation of TNF RNA in a separate in vitro stimulation experiment (FIG. 9).

These data demonstrate that the binding protein AbS does not induce either IL21-responsive or cytokine storm-associated signals, and represents a promising target for drug development.

While several of the specific examples described herein were studies using AbS, the same or similar types of studies can be done with any anti-IL21R binding proteins, such as those incorporated within the present application or other anti-IL21R binding proteins/antibodies, to determine the effects of the particular IL21R binding protein/antibody on, e.g., cytokine storm, and to assist in evaluating the safety of particular anti-IL21R binding proteins/antibodies in human therapeutics. For example, such experiments may be performed for inclusion in regulatory submissions and used to evaluate future anti-IL21R therapeutics.

### EQUIVALENTS

Those skilled in the art will recognize, or be able to ascertain using no more than routine experimentation, many equivalents of the specific embodiments of the invention described herein. Such equivalents are intended to be encompassed by the following claims.

What is claimed is:

1. A method of predicting whether a therapeutic binding protein will induce a cytokine storm upon administration to a first mammalian subject comprising the steps of:
(a) administering the therapeutic binding protein to a second mammalian subject, wherein the second mammalian subject is a binding protein-treated second mammalian subject;
(b) obtaining a blood sample from the binding protein-treated second mammalian subject;
(c) determining the level of expression of at least one cytokine storm gene in the blood of the binding protein-treated second mammalian subject; and
(d) comparing the level of expression of the at least one cytokine storm gene in the blood of the binding protein-treated second mammalian subject to the level of expression of the at least one cytokine storm gene in the blood of an untreated second mammalian subject,
wherein a level of expression of the at least one cytokine storm gene in the binding protein-treated second mammalian subject substantially greater than the level of expression of the at least one cytokine storm gene in an untreated second mammalian subject indicates that the therapeutic binding protein will induce a cytokine storm in the first mammalian subject.

2. The method of claim 1, wherein the first mammalian subject is a human subject.

3. The method of claim 1, wherein the therapeutic binding protein is an anti-IL21R binding protein.

4. The method of claim 3, wherein the anti-IL21R binding protein is AbS.

5. The method of claim 2, wherein the second mammalian subject is a member of a safety study species.

6. The method of claim 5, wherein the member of the safety study species is a cynomolgus monkey subject.

7. The method of claim 1, wherein the at least one cytokine storm gene is selected from the group consisting of: IL4, IL2, IL1β, IL12, TNF, IFNγ, IL6, IL8, and IL10.

8. The method of claim 1, wherein the method comprises determining the levels of expression or at least two, at least three, at least four, at least five, at least six, at least seven, at least eight, or at least nine cytokine storm genes.

9. The method of claim 8, wherein the method comprises determining the levels of expression of nine cytokine storm genes.

10. The method of claim 1, wherein the method of determining the level of expression of at least one cytokine storm gene in the blood of the binding protein-treated second mammalian subject comprises measuring the level of mRNA expression of the at least one cytokine storm gene.

11. The method of claim 1, wherein the method of determining the level of expression of at least one cytokine storm gene in the blood of the binding protein-treated second mammalian subject comprises measuring the level of protein expression of the at least one cytokine storm gene.

12. The method of claim 11, wherein measuring the level of protein expression of at least one cytokine storm gene comprises measuring the level of cytokine release of the at least one cytokine storm gene.

13. A method of predicting whether a therapeutic binding protein will induce a cytokine storm in a mammalian subject comprising the steps of:
(a) obtaining a blood sample from the mammalian subject;
(b) incubating the therapeutic binding protein with the blood sample, wherein the blood sample is a binding protein-treated blood sample;
(c) determining the level of expression of at least one cytokine storm gene in the binding protein-treated blood sample; and
(d) comparing the level of expression of the at least one cytokine storm gene in the binding protein-treated blood sample to the level of expression of the at least one cytokine storm gene in an untreated or a negative control-treated blood sample,
wherein a level of expression of the at least one cytokine storm gene in the binding protein-treated blood sample substantially greater than the level of expression of the at least one cytokine storm gene in the untreated or negative control-treated blood sample indicates that the therapeutic binding protein will induce a cytokine storm in the mammalian subject.

14. The method of claim 13, wherein the mammalian subject is a human subject.

15. The method of claim 13, wherein the mammalian subject is a member of a safety study species.

16. The method of claim 15, wherein the member of the safety study species is a cynomolgus monkey subject.

17. The method of claim 13, wherein the blood sample is a purified peripheral blood mononuclear cell (PBMC) sample.

18. The method of claim 13, wherein the therapeutic binding protein is an anti-IL21R binding protein.

19. The method of claim 18, wherein the anti-IL21R binding protein is AbS.

20. The method of claim 13, wherein the at least one cytokine storm gene is selected from the group consisting of: IL4, IL2, IL1β, IL12, TNF, IFNγ, IL6, IL8, and IL10.

21. The method of claim 13, wherein the method comprises determining the levels of expression or at least two, at least three, at least four, at least five, at least six, at least seven, at least eight, or at least nine cytokine storm genes.

22. The method of claim 21, wherein the method comprises determining the levels of expression of nine cytokine storm genes.

23. The method of claim 13, wherein the method of determining the level of expression of at least one cytokine storm gene in the binding protein-treated blood sample comprises measuring the level of mRNA expression of the at least one cytokine storm gene.

24. The method of claim 13, wherein the method of determining the level of expression of at least one cytokine storm gene in the binding protein-treated blood sample comprises measuring the level of protein expression of the at least one cytokine storm gene.

25. The method of claim 24, wherein measuring the level of protein expression of the at least one cytokine storm gene comprises measuring the level of cytokine release of the at least one cytokine storm gene.

26. A method of determining whether an anti-IL21R binding protein is a neutralizing anti-IL21R binding protein comprising the steps of:
(a) contacting a first blood sample from a subject with an IL21 ligand;
(b) determining a level of expression of at least one IL21-responsive gene in the first blood sample contacted with the IL21 ligand;
(c) contacting a second blood sample from the subject with the IL21 ligand in the presence of an anti-IL21R binding protein;
(d) determining the level of expression of the at least one IL21-responsive gene in the second blood sample contacted with the IL21 ligand in the presence of the anti-IL21R binding protein; and
(e) comparing the levels of expression of the at least one IL21-responsive gene determined in steps (b) and (d),
wherein a change in the level of expression of the at least one IL21-responsive gene indicates that the anti-IL21R binding protein is a neutralizing binding protein.

27. The method of claim 26, wherein the subject is a mammal.

28. The method of claim 27, wherein the subject is a monkey.

29. The method of claim 27, wherein the subject is a human.

30. The method of claim 26, wherein the at least one IL21-responsive gene is selected from the group consisting of CCL19, CCL2, CCL3, CCR2, CD19, CD40, CSF2, CSF3, CXCL10, CXCL11, GZMB, IFNγ, IL10, IL12β, IL1β, IL2RA, IL6, PRF1, PTGS2, and TBX21.

31. The method of claim 30, wherein the at least one IL21-responsive gene is IL2RA.
